(19) United States
(12) Patent Application Publication
(10) Pub. No.: US 2023/0218746 A1
(43) Pub. Date: Jul. 13, 2023

(54) COMPOSITIONS AND METHODS RELATING TO ANTIVIRAL THERAPEUTICS

(71) Applicant: NORTH CAROLINA STATE UNIVERSITY, Raleigh, NC (US)

(72) Inventors: Ke Cheng, Raleigh, NC (US); Phuong-Uyen Cao Dinh, Raleigh, NC (US); Zhenhua Li, Raleigh, NC (US)

(21) Appl. No.: 18/001,561

(22) PCT Filed: Jun. 15, 2021
(86) PCT No.: PCT/US21/37484

§ 371 Date: Dec. 12, 2022

Related U.S. Application Data
(60) Provisional application No. 63/070,888, filed on Aug. 27, 2020.
(60) Provisional application No. 63/039,598, filed on Jun. 16, 2020.

(51) Int. Cl. A61K 39/215; A61K 35/42; A61K 38/48; A61K 38/17; A61K 9/127; A61K 31/675; A61K 38/21; A61K 31/513; A61K 31/427; A61P 31/14

(52) U.S. Cl. CPC A61K 39/215; A61K 35/42; A61K 38/4813; A61K 38/1709; A61K 38/177; A61K 9/127; A61K 31/675; A61K 38/215; A61K 31/513; A61K 31/427; A61P 31/14; A61K 2039/5156

(57) ABSTRACT

The present disclosure provides compositions and methods related to antiviral therapeutics. In particular, the present disclosure provides novel compositions and methods for treating and/or preventing viral infections using vesicles derived from lung spheroid cells (LSCs). LSC-derived vesicles can be used as viral decoy nanoparticles for therapeutic applications, as virus-like particles (VLPs) for vaccine production, and as an antiviral drug delivery platform.

### CROSS REFERENCE TO RELATED APPLICATION

This application claims priority to and the benefit of U.S. Provisional Patent Application Ser. No. 63/039,598 filed Jun. 16, 2020, and U.S. Provisional Patent Application Ser. No. 63/070,888, filed Aug. 27, 2020, both of which are incorporated herein by reference in their entireties and for all purposes.

### FIELD

The present disclosure provides compositions and methods related to antiviral therapeutics. In particular, the present disclosure provides novel compositions and methods for treating and/or preventing viral infections using vesicles derived from lung spheroid cells (LSCs). As provided herein, LSC-derived vesicles can be used as viral decoy nanoparticles for therapeutic applications, as virus-like particles (VLPs) for vaccine production, and as an antiviral drug delivery platform.

### BACKGROUND

Severe acute respiratory syndrome coronavirus 2 (SARS-CoV-2), the pathogen at the center of the current global pandemic, is the cause of coronavirus disease-2019 (COVID-19). Coronaviruses are considered common viruses; alpha (α-) coronavirus and beta (β-) coronavirus can infect mammals and often manifest as the common cold or gastrointestinal (GI) discomfort. Rarely, more severe and lethal forms emerge, such as SARS-CoV-2, which is capable of infecting the respiratory and immune systems and inducing secretion of pro-inflammatory cytokines triggering an increase in alveolar edema, hypoxemia, dyspnea, and systemic inflammatory response syndrome (SIRS). Like its predecessors, SARS-CoV-1 (cause of SARS in 2003) and MERS-CoV (cause of MERS in 2012), SARS-CoV-2 is an enveloped, positive-sense, p-coronavirus with dangerously high human-to-human transmission rates, with reported RO ranging from 2-6. Initial efforts to combat the virus were primarily centered on containment to stop the spread and to elucidate its pathogenesis; however, the virus continues to circulate, claiming over 350,000 lives worldwide. It is becoming increasingly evident that not only is an efficacious vaccine necessary, but also therapeutic treatment options are required for controlling the spread of the virus and preventing subsequent waves of infection. Unfortunately, no therapy has yet been approved, and treatment remains mainly focused on palliative and symptomatic management. It is becoming undeniably evident that in addition to an efficacious vaccine, the development of therapeutics is necessary for completely ending this pandemic and providing a solution to COVID-19 patients who are severely ill. Researchers around the world are in an urgent race to find an effective therapy for COVID-19. According to the published interim results from the World Health Organization's Solidarity Trial on 15 Oct. 2020, all 4 of the evaluated treatments (remdesivir, hydroxychloroquine, lopinavir/ritonavir, and interferon) had little or no effect on the overall mortality, necessity for mechanical ventilation, and length of hospital stay in hospitalized COVID-19 patients.

### SUMMARY

Embodiments of the present disclosure include a composition comprising a plurality of nanovesicles derived from a cell comprising at least one cell surface protein capable of binding a virus.

In some embodiments, the cell is a lung spheroid cell (LSC). In some embodiments, the at least one cell surface protein comprises Angiotensin-converting enzyme 2 (ACE2), or a derivative or fragment thereof. In some embodiments, the ACE2 protein or derivative or fragment thereof is endogenous to the cell. In some embodiments, the ACE2 protein or derivative or fragment thereof is exogenous to the cell.

In some embodiments, the at least one cell surface protein further comprises AQP5, SFTPC, CD68, EpCAM, CD90, and/or MUC5b.

In some embodiments, the plurality of nanovesicles comprise an average size ranging from about 50 nm to about 1000 nm. In some embodiments, the plurality of nanovesicles comprise an average size of about 320 nm.

In some embodiments, the composition further comprises at least one pharmaceutically-acceptable excipient or carrier.

In some embodiments, the virus is a coronavirus. In some embodiments, the coronavirus is selected from the group consisting of 229E, NL63, OC43, HKU1, MERS-CoV, SARS-CoV, and SARS-CoV-2.

In some embodiments, the plurality of nanovesicles comprise at least one therapeutic protein, peptide, polypeptide, nucleic acid molecule, polynucleotide, mRNA, siRNA, miRNA, antisense oligonucleotide, drug, or therapeutic small molecule.

Embodiments of the present disclosure also include a method of treating a viral infection comprising administering any of the compositions described above to a subject in need thereof.

In some embodiments, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration. In some embodiments, the composition is administered via nebulization to lung tissue. In some embodiments, administration of the plurality of nanovesicles reduces viral load in the subject. In some embodiments, the composition is administered at a dosage ranging from about 1×10⁸ to about 1×10¹² particles per kg of body weight of the subject.

Embodiments of the present disclosure also includes a method of generating a plurality of nanovesicles capable of treating a viral infection. In accordance with these embodiments, the method includes culturing a plurality of lung spheroid cells (LSCs), and subjecting the plurality of LSCs to an extrusion process to produce the plurality of nanovesicles.

In some embodiments, the extrusion process comprises passing the LSCs through an extruder comprising 5 μm 1 μm, and 400 nm pore-sized membrane filters.

In some embodiments, the method further comprises purifying and concentrating the plurality of nanovesicles using ultrafiltration.

Embodiments of the present disclosure also includes a composition comprising a plurality of exosomes derived from lung spheroid cells (LSCs). In accordance with these embodiments, the composition includes a plurality of LSC exosomes comprising (i) at least one membrane-associated protein on the surface of the plurality of LSC exosomes, and/or (ii) at least one antiviral therapeutic agent contained within the plurality of LSC exosomes.

In some embodiments, the at least one membrane-associated protein on the surface of the plurality of LSC exosomes comprises a viral-specific protein. In some embodiments, the viral-specific protein comprises a Spike protein (S protein). In some embodiments, the at least one antiviral therapeutic agent contained within the plurality of LSC exosomes comprises mRNA encoding the S protein.

In some embodiments, the at least one membrane-associated protein on the surface of the plurality of LSC exosomes comprises a protein capable of binding a virus.

In some embodiments, the protein capable of binding a virus comprises Angiotensin-converting enzyme 2 (ACE2), or a derivative or fragment thereof.

In some embodiments, the at least one antiviral therapeutic agent contained within the plurality of LSC exosomes comprises Remdesivir.

In some embodiments, the composition further comprises at least one pharmaceutically-acceptable excipient or carrier.

Embodiments of the present disclosure also include a method of preventing a viral infection comprising administering any of the compositions described above to a subject.

In some embodiments, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration. In some embodiments, the composition is administered via nebulization to lung tissue. In some embodiments, the virus is a coronavirus. In some embodiments, the coronavirus is selected from the group consisting of 229E, NL63, OC43, HKU1, MERS-CoV, SARS-CoV, and SARS-CoV-2.

### BRIEF DESCRIPTION OF THE DRAWINGS

FIGS. 1A-1M: Characterizations of lung spheroid cell-derived nanodecoys. (A) Representative confocal images of LSCs labeled with ACE2, AQP5, and SFTPC antibodies. Scale bars, 20 μm. (B) Representative flow cytometry analysis of LSCs (B) and EDCs (C) for ACE2 expression and (D) quantitative results of flow cytometry analysis of EDCs and LSCs for ACE2, EpCAM, CD90, MUC5b, and vWF. Data are shown as mean±SD, n=4 or 6 independent experiments. Statistical analysis was performed by two-way ANOVA with a Tukey post hoc test. See FIG. 32 for gating strategies. (E) Size measurement of nanodecoys using Nanosight. (F) Western blot of Alix and Calnexin in LSC-nanodecoys and LSCs. Flow cytometry analysis showing the expressions of ACE2 (G) and type II pneumocytes maker SFPTC (H) on LSC-nanodecoys. See FIG. 32 for gating strategies. (I) Measurement of ACE2 numbers on both cells and nanodecoys. HEK indicates HEK293. Data are shown as mean±SD, n=3 independent experiments. Transmission electron microscopy (TEM) images showing naked nanodecoys (J) and enlarged FIG. 1K. TEM images showing spike S1-bound nanodecoys (L) and enlarged FIG. 1M. Spike S1 was detected using gold nanoparticle-labeled secondary antibodies with diameters of 10 nm. Cartoon pictures (insets in FIGS. 1J and 1L) were created with BioRender.com.

FIGS. 2A-2L: Neutralization of spike S1 by nanodecoys. (A) Dose-dependent neutralization of spike S1 by LSC-nanodecoys or HEK-nanodecoys. Data are shown as mean±SD, n=3 independent experiments. (B) Schematic illustrating the experimental design. (C) Interaction of spike S1 (red) and nanodecoys (white) when co-cultured with lung cells (green). (D) Schematic illustrating the experimental design. (E) Representative confocal images showing internalization of nanodecoys by macrophages (CD4, red). (F) Schematic illustrating the co-culture experiment and (G) confocal images of the internalization of nanodecoys by macrophages co-cultured with lung cells (CD90, green). At least three images were taken per group. Flow cytometry analysis showing internalization of DiD-labeled nanodecoys by LSCs (I) and macrophages (K) and (L) its corresponding quantitation. PBS was used as control group for LSCs (H) and macrophages (J). See FIG. 33 for gating strategies. Data are shown as mean±SD, n=3 independent experiments. Statistical analysis was performed with two-tailed Student t-test. Scale bars, 50 μm for FIGS. 2C, 2E, and 2G. Cartoon pictures were created with BioRender.com.

FIGS. 3A-3N: Neutralization of SARS-CoV-2 mimicking viruses by nanodecoys. (A) The synthesis of activated NTA and the chemical structure. (B) Schematic illustrating the modification of lentivirus with spike S1 to generate a SARS-CoV-2 mimic. TEM images showing lentivirus (C), SARS-CoV-2 mimic (D), and spike S1 on lentivirus using gold nanoparticle-labeled secondary antibodies with diameters of 10 nm (E), in which SARS-CoV-2 mimicking viruses (yellow arrows) were attached to a nanodecoy (dotted circle). Scale bars, 100 nm for FIGS. 3C-3E. (F) Neutralization assay of SARS-CoV-2 mimics by nanodecoys. Data are shown as mean±SD, n=4 independent experiments. (G) Schematic showing the experimental design. (H) Nanodecoys (white) neutralize SARS-CoV-2 mimics (red) in a co-culture with lung cells (green) and macrophages. Scale bar, 50 μm. (I-L) Representative confocal images (at least three images were taken per animal) and (M) flow cytometry analysis showing nanodecoys inhibit the SARS-CoV-2 mimic virus (red) entry into the lung cells (green). Scale bars, 50 μm. (N) Corresponding quantitation from (M). See FIG. 33 for gating strategies. Data are shown as mean±SD, n=3 independent experiments. Statistical analysis was performed by one-way ANOVA with a Tukey post hoc test. Cartoon pictures were created with BioRender.com.

FIGS. 4A-4E: Biodistribution of nanodecoys after inhalation. (A) Schematic showing experimental design of nanodecoy inhalation in CD1 mice. Created with BioRender.com. (B) Corresponding quantitative results from (C) of DiD-labeled nanodecoys in heart, lung, liver, kidney, and spleen tissues. Data are shown as mean±SD, n=3 animals. (C) Representative confocal images of DiD-labeled nanodecoys (red) in tissue sections. (D) Representative confocal images showing nanodecoys in lung tissues co-localizing with lung cells (AQP5, SFTPC) and macrophages (CD68) 24 hrs post-inhalation. (E) Quantification of the percent of nanodecoy-positive macrophages. Data are shown as mean±SD, n=3 animals. Scale bars, 200 μm for FIGS. 4C-4D.

FIGS. 5A-5J: Nanodecoy inhalation accelerates the clearance of the SARS-CoV-2 mimic viruses in a mouse model. (A) Schematic showing the animal study design. Created with BioRender.com. (B) Representative ex vivo IVIS imaging of lung tissues from mice with various treatments. n=3 animals per group. (C) Quantification of fluorescence intensities of SARS-CoV-2 mimics from the imaging data in (B). Data are shown as mean±SD, n=3 animals per group. Statistical analysis was performed by two-way ANOVA with a Tukey post hoc test for multiple comparisons. (D) Representative confocal images of AF647-labeled SARS-CoV-2 mimics (red) in lung sections. Scale bar, 50 μm.(E) Corresponding semi-quantitative analysis of AF647-labeled SARS-CoV-2 mimics in lung tissues. Data are shown as mean±SD, n=3 animals per group. Statistical analysis was performed two-way ANOVA with a Tukey post hoc test for multiple comparisons. (F-J) Cytokine array analysis of various inflammatory cytokines in the serum 3-days after treatment.

FIGS. 6A-6I: LSC-nanodecoy inhalation treats SARS-CoV-2 infection in cynomolgus macaques. (A) Schematic depicting the cynomolgus macaque study design. Created with BioRender.com. (B and C) Viral subgenomic RNA (sgRNA) copies/Swab in nasal swabs (NS) and bronchoalveolar lavage (BAL) at various timepoints following challenge. Each dot represents data from one animal. n=3 animals per group. (D) Representative H&E images of fixed lung tissues from SARS-CoV-2 infected cynomolgus macaques and at least three images were taken per animal. Top: scale bar, 500 μm; bottom: scale bar, 100 μm. (E) Representative images of SARS nucleocapsid (SARS-N) immunohistochemistry (IHC) staining in fixed lung tissues from SARS-CoV-2 infected cynomolgus macaques treated with control or LSC-nanodecoys 8-days post-viral challenging. Top: Scale bar, 100 μm; bottom: scale bar, 20 μm. (F) Quantification of lung fibrosis of infected cynomolgus macaques by Ashcroft scoring; each dot represents data from one animal; data are shown as mean±SD, n=3 animals per group. Statistical analysis was performed by two-tailed Student t-test. Ashcroft scoring was performed blindly. G) Quantitation of positive SARS nucleocapsid numbers in lung tissues of infected cynomolgus macaques. Each dot represents data from one animal; data are shown as mean±SD, n=3 animals per group. Statistical analysis was performed with two-tailed Student t-test. (H) Representative images of RNAscope in situ hybridization detection of vRNA in infected cynomolgus macaques. ZIKA as a control probe. Scale bar, 100 μm. (I) Representative immunofluorescence images of SARS-N (red), pan-CK (green), Iba-1 (greyscale), CD68 (green) CD206 (magenta) and DAPI (blue). Scale bar, 50 μm. At least three images were taken per animal.

FIGS. 7A-7F: Characterization of ACE2⁺ exosomes. (A) Immunofluorescence of ACE2 expression in lung spheroid cell (LSC) and HEK293T (HEK) parental control cells. (B) Quantification of ACE2 expression in FIG. 7A. (C) TEM images of LSC derived exosomes (LSC-Exo) and HEK cells derived exosomes (HEK-Exo). (D) Nanoparticle tracking analysis of LSC-Exo and HEK-Exo.(E) Flow profiles of ACE2 expression in LSC, HEK, LSC-Exo and HEK-Exo. (F) Corresponding quantification of ACE2 expression in FIG. 7E.

FIGS. 8A-8F: LSC-Exo inhibits SARS-CoV-2 pseudovirus infection to human host cells. Flow plots (A) and quantification (B) of pseudovirus-infected A549-ACE2 cells, detected with GFP reporter expression which was inhibited by LSC-Exo. (C) Confocal imaging of A549-ACE2 cells incubated with SARS-CoV-2-GFP pseudovirus and ACE2, or HEK-Exo or LSC-Exo, respectively. Phalloidin (red), SARS-CoV-2-GFP (green). (D) Ex-vivo imaging of mouse lungs from each group at 24 hours post-inoculation, in which mice were inhaled with ACE2 protein, HEK-Exo or LSC-Exo at −2 hour, followed by intranasal inoculation of SARS-CoV-2-GFP pseudovirus. (E) Corresponding semi-quantitative analysis of SARS-CoV-2-GFP pseudovirus in lung tissues in FIG. 8D. (F) Representative immunostaining of whole lung, tracheal, bronchial, and parenchymal sections for DAPI (blue), Phalloidin (red), and SARS-CoV-2 pseudovirus (green). These images were obtained under magnification of 4×.

FIGS. 9A-9E: LSC-Exo protects SARS-CoV-2 infection in hamsters. (A) Schematic showing animal study design. (B) Impact of LSC-Exo protection on viral gRNA in oral swabs (OS) at the indicated time points. (C) Impact of LSC-Exo protection on viral genomic RNA (gRNA) in bronchoalveolar lavage (BAL) fluid 7 days post-challenge. (D) Representative H&E and Masson's trichrome images of lung tissues from hamsters at 7 days post-challenge. (E) Representative images of RNAscope in situ hybridization detection of vRNA in lung tissues of hamsters 7 days post-challenge.

FIGS. 10A-10K: RFP-Loaded LSC-Exosomes have superior distribution to the lung. (A) The experimental schematic of RFP-Loaded LSC-Exosomes and RFP-Loaded Liposomes in healthy CD1 mice; n=3 per group. (B) Ex-vivo imaging of mouse lungs after RFP-Loaded LSC-Exosome or RFP-Loaded Liposome delivery after 4 and 24 hours. (C) Quantification of the integrated density of RFP fluorescence in ex-vivo mouse lungs; each dot represents data from one lung; n=3 per group. (D) Representative immunostaining of whole lung, tracheal, bronchial, and parenchymal sections for DAPI (blue), Phalloidin (green), and exosomes or liposomes (red). These images were obtained under magnification of 10. (E) Quantification of the integrated density of RFP fluorescence across all groups in tracheal, bronchial, and parenchymal tiles from whole lung images; each dot represents data from one image tile; n=12-276. (F-H) Quantification of the integrated density of RFP fluorescence in tracheal (F), bronchial (G), and parenchymal (H) tiles from whole lung images; each dot represents data form one image tile; n=2-82. (I) Representative immunostaining of parenchymal sections for DAPI (blue), CD11b (green), and exosomes or liposomes (red). These images were obtained under magnification of 60. (J) Quantification of exosome orliposome uptake by CD11b+ APCs in ex-vivo mouse lungs; numbers in red indicate total number of positive cells across all representative images; n=6 images per group. Throughout, data are mean±s.d. P-value as indicated by one-way ANOVA followed by post hoc Bonferroni correction. * indicates p<0.05; ** p<0.01; *** p<0.001; **** p<0.0001. (K) Representative schematic of inhalation of RBD-Exo VLP vaccine that induces the neutralization of SARS-CoV-2 in hamsters and protects the lungs.

FIGS. 11A-11K: Characterizations of RBD-Exo and stability studies. (A) Schematic illustrating the modification of LSC-Exo with RBD to generate RBD-Exo. (B) Transmission electron microscopy (TEM) images of LSC-Exo and RBD-Exo. RBD were detected using gold nanoparticle-labeled secondary antibodies with diameters of 15 nm. (C) Immunoblots of RBD and CD63 in lysed RBD-Exo, RBD, and Exo. (D) Size measurement of LSC-Exo and RBD-Exo via nanoparticle tracking analysis. (E) Representative TEM images, (F) size change, (G) total number and (H) RBD level change of RBD-Exo after storing at −80° C., 4° C. and RT for 21 days, respectively. RBD level was calculated by the ratio of treatment group and pre-lyophilization (Pre-lyo). (I) Summary of stability data of RBD-Exo over 21 days. (J) Representative immunostaining of RAW264.7 cells for DAPI (blue) and RBD or RBD-Exo (red). Scale bar, 50 sm. (K) Flow cytometry analysis of RBD and RBD-Exo internalization by RAW264.7 cells. Data are mean±s.d. P-value as indicated by unpaired t tests. *** indicates p<0.001.

FIGS. 12A-12F: RBD-Exo vaccination induces antibody production and enhances the clearance of SARS-CoV-2 mimics in mice. (A) Schematic showing animal study design. (B) Ex vivo fluorescent imaging of lungs after inhalation of SARS-CoV-2 mimics at different time points, one week after the second vaccination. (C) Corresponding semi-quantitative analysis of AF647-labeled SARS-CoV-2 mimics in lung tissues from confocal images lung sections. (D) Anti-RBD antibody titer from murine serum detected by ELISA. RBD-specific secretory IgA (SIgA) antibody titers from nasopharyngeal lavage fluid (NPLF) (E) and bronchoalveolar lavage fluid (BALF) (F) detected by ELISA. Throughout, data are mean±s.d. P-value as indicated by unpaired t tests. * indicates p<0.05; ** p<0.01; *** p<0.001. ns indicates not significant.

FIGS. 13A-13D: Induction of systemic cytokines in RBD-Exo vaccinated mice. (A) Representative images of IFN-7 release spots in 96-well plate in the presence of RBD with 10⁶ splenocytes/well. Splenocytes derived from each treatment group that received intravenous (IV) and nebulization (N) administration. (B) IFN-γ splenocytes expressed as spot forming units (SFU) per 10⁶ cells. (C) TNF-α levels from splenocytes supernatant re-stimulated by RBD. (D) IL-6 splenocytes levels from splenocytes supernatant re-stimulated by RBD. Throughout, data are mean±s.d. P-value as indicated by unpaired t tests. * indicates p<0.05; ** p<0.01; *** p<0.001; **** p<0.0001. ns indicates not significant.

FIGS. 14A-14G: Protective effect of RBD-Exo vaccine in the Syrian hamster model of SARS-CoV-2 infection. (A) Overview of experimental design. (B) Impact of RBD-Exo on viral genomic RNA (gRNA) in bronchoalveolar lavage (BAL) fluid 7 days post-challenge. (C) Impact of RBD-Exo on viral gRNA in oral swabs (OS) at the indicated time points. (D) RBD-specific binding antibody from hamster serum at week 2 (before challenge) detected by ELISA. Representative H&E (E) and Masson's trichrome (F) images of lung tissues from hamsters at 7 days post-challenge. Top: scale bar, 500 μm; bottom: scale bar 100 μm. (G) Quantitation of lung fibrosis of challenged hamsters by Ashcroft scoring; each dot represents data from one animal; Ashcroft scoring analysis was performed blindly. Throughout, data are mean±s.d. P-value as indicated by One-way ANOVA. * indicates p<0.05; ** p<0.01; *** p<0.001; **** p<0.0001.

FIGS. 15A-15G: Histopathological changes and RNAscope analysis in Syrian hamsters vaccinated with RBD-Exo. (A) Representative images of SARS nucleocapsid (SARS-N) immunohistochemistry (IHC) staining in fixed lung tissues from hamsters vaccinated with PBS, RBD or RBD-Exo 7 days post viral challenging. Scale bar, 100 μm (B) Representative immunofluorescence images of SARS-N (magenta), pan-CK (green) and DAPI (blue) of lung tissues in hamsters to investigate the distribution of SARS-N. Scale bar, 50 μm. (C) Representative images of RNAscope in situ hybridization detection of vRNA in lung tissues of hamsters 7 days post-challenge. Scale bar, 100 μm. (D) Representative immunofluorescence images of SARS-N (greyscale), Ibal-1 (red), CD206 (green) and DAPI (blue) of lung tissues in hamsters 7 days post challenge. Scale bar, 50 μm. (E) Representative images of CD3 T lymphocytes, MPO and Interferon inducible gene MX1 IHC staining of hamsters 7 days post challenge. Scale bar, 50 μm. (F) Quantitation of positive SARS-N cell numbers in lung tissues of hamsters. Each dot represents data from one image file, n=15. (G) Quantitation of positive CD3, MPO and MX1 cell numbers in lung tissues of hamsters, respectively. Each dot represents data from one image file, n=15. Throughout, data are mean±s.d. P-value as indicated by one-way ANOVA. * indicates p<0.05; ** p<0.01; *** p<0.001; **** p<0.0001.

FIG. 16: Schematic illustrating the generation of nanodecoys from lung spheroid cells to inhalable nanodecoys and potential nanodecoy therapy for SARS-CoV-2 infection. Created with BioRender.com.

FIG. 17: Confocal images showing lung explant derived cells (EDCs) labeled with ACE2, AQP5, and SFTPC antibodies. Scale bar, 20 μm.

FIGS. 18A-18C: Immunoblotting and flow cytometry of HEK293 cells, human fibroblasts, human lung explant-derived cells (EDCs) and human lung spheroid cells (LSCs) for ACE2 expression. (A) Western blots of HEK293, human lung fibroblasts, EDCs, and LSCs. (B) Quantitative results from (A). Data are shown as mean±SD, n=3 independent experiments. (C) Flow cytometry analysis of ACE2 positive percentages in HEK293 cells, human lung fibroblasts, EDCs, LSCs, LSC-Exosomes and LSC-nanodecoys. Data are shown as mean±SD, n=3 or 6 independent experiments.

FIGS. 19A-19E: Flow cytometry characterizations of human LSCs. See FIG. 32 for gating strategies.

FIG. 20: Double-stain flow cytometry characterizations of ACE2⁺ LSCs. See FIG. 32 for gating strategies.

FIG. 21: Internalization of nanodecoys by macrophages. Left, confocal images showing internalization of nanodecoys by macrophages derived from peripheral blood or lung tissues. Right, quantitative results on internalization. Data are shown as mean±SD, n=4 independent experiments. Scale bar, 50 μm.

FIG. 22: A representative confocal image showing SARS-CoV-2 mimic internalized by LSCs. Scale bar, 20 μm.

FIG. 23: Confocal imaging and flow cytometry analysis showing internalization of lentivirus and NTA-tagged lentivirus. See FIG. 33 for gating strategies.

FIGS. 24A-24B: Nanodecoys block viral entry of SARS-CoV-2 mimics. (A) Flow cytometry analysis showing that the nanodecoys blocked virus entry into lung cells in a dose-dependent manner and (B) corresponding quantitative results from (A). Data are shown as mean±SD, n=3 independent experiments. Statistical analysis was performed with a one-way ANOVA with a Tukey post hoc test. See FIG. 33 for gating strategies.

FIG. 25: Biodistribution of inhaled nanodecoys. Ex vivo fluorescent images of major organs at various time points after inhalation of LSC-nanodecoys.

FIG. 26: Nanodecoy inhalation does not trigger inflammation in the lungs. Left, representative confocal images showing CD68 positive cells in lung tissues at various time points after inhalation of LSC-nanodecoys. Right, quantitative results from images on the left. Data are shown as mean±SD, n=3 animals per group and five images were taken per group. Statistical analysis was performed by one-way ANOVA with a Tukey post hoc test. Scale bar, 200 μm.

FIG. 27: Unmerged FIG. 5D. Representative confocal images showing LSC-nanodecoys in lung tissues co-localizing with lung cells (AQP5, SFTPC) and macrophages (CD68) 24 hrs post-inhalation. Scale bar, 200 μm.

FIG. 28: Pathological studies on the toxicity of LSC-nanodecoy inhalation therapy. H&E staining of major organs 14 days after LSC-nanodecoy treatment. Scale bars, 560 μm (40×) and 110 μm (200×).

FIG. 29: Hematology and biochemistry studies on nanodecoy toxicity in mice 14 days after nanodecoy inhalation. Data are shown as mean±SD, n=3 animals per group.

FIG. 30: Hematology studies in cynomolgus macaques after LSC-nanodecoy therapy. Data are shown as mean±SD, n=3 animals per group.

FIG. 31: Percent of weight and temperature changes after SARS-CoV-2 challenge in individual cynomolgus macaques. n=3 animals per group.

FIG. 32: Flow cytometry gating strategies for experiments in FIGS. 1B-1C, 1G-1H, and FIGS. 19-20.

FIGS. 33A-33B: Flow gating strategies for experiments in FIGS. 2I-2L (A) and FIGS. 3M-3N (B), and FIGS. 23-24 (B).

FIG. 34: Representative immunostaining of DAPI (blue) and AF647-labeled SARS-CoV-2 mimics (red) in lung sections from mice sacrificed 2 days after viral challenge.

FIG. 35: Representative immunostaining of DAPI (blue) and AF647-labeled SARS-CoV-2 mimics (red) in lung sections from mice sacrificed 6 days after viral challenge.

FIG. 36: Flow cytometry analysis of dendritic cells (DCs) expressing co-stimulatory molecule CD86 in splenocytes derived from vaccinated mice after re-stimulation by RBD.

FIG. 37: Flow cytometry analysis of dendritic cells (DCs) expressing CD40 in splenocytes derived from vaccinated mice after re-stimulation by RBD.

FIG. 38: Flow cytometry analysis of dendritic cells (DCs) expressing CD80 in splenocytes derived from vaccinated mice after re-stimulation by RBD.

FIG. 39: Clinical chemistry and hematological parameters from the peripheral blood of hamsters 7 days post challenge. Each dot represents data from one animal. The grey area represents the clinical chemistry and hematological range of normal hamsters.

### DETAILED DESCRIPTION

Embodiments of present disclosure provide compositions and methods related to antiviral therapeutics. In particular, the present disclosure provides novel compositions and methods for treating and/or preventing viral infections using vesicles derived from lung spheroid cells (LSCs). As provided herein, LSC-derived vesicles can be used as viral decoy nanoparticles for therapeutic applications, as virus-like particles (VLPs) for vaccine production, and as an antiviral drug delivery platform.

Viral Decoy Nanoparticles. Angiotensin-converting enzyme 2 (ACE2), which is present on many cell types and found in almost all tissues, is a carboxypeptidase that has been shown to play a pivotal role in host cell viral entry. SARS-CoV-2 specifically attacks ACE2 presenting respiratory type II pneumocytes in the lungs and goblet secretory cells in the nasal mucosa as its primary sites of infection. In the present disclosure, the virus's cell entry was exploited as a Trojan horse strategy. As demonstrated by previous studies, Lung Spheroid Cells (LSCs) have been developed, from initial rodent studies to an on-going Phase 1 clinical trial (NCT04262167), as a cell therapy to treat lung fibrosis and inflammation. LSCs are a mixture of resident lung epithelial (containing both types I and II pneumocytes) and mesenchymal cells. As resident lung cells, they express ACE2. Based on this, LSC membrane nanovesicles were generated as ACE2 nanodecoys. Those nanodecoys, acting as cell mimics, are capable of binding SARS-CoV-2 Spike (S-) protein and triggering a response from macrophages for viral elimination.

Therapeutic antibodies and fusion inhibitors have been developed for targeting the spike protein of SARS-CoV-2. However, more aggressive variants associated with the mutations in the spike protein of SARS-CoV-2 have been discovered. Therefore, antiviral strategies based on the human receptor ACE2, used by the virus to gain host cell entry rather than the viral components, will experience greater interest since no mutations are to be expected on the host cells. The results of the present disclosure provide a non-invasive therapeutic strategy for neutralizing SARS-CoV-2. This approach is fundamentally different from the current two strategies: antiviral drugs and vaccines. The LSCs used to fabricate the nanodecoy are generated through a robust, reproducible, and scalable culturing method suitable for producing clinically applicable quantities of cell therapy products. Moreover, this nanodecoy technology is highly translatable as the parental cells are currently in the early clinical trial stage as a potential treatment for pulmonary fibrosis.

As described further herein, embodiments of the present disclosure provide the first evidence in a nonhuman primate model of live SARS-CoV-2 infection that cell-derived and cell-mimicking nanodecoys can protect lung cells from the infections and damages from SARS-CoV-2. The cynomolgus macaque model recapitulates many clinical features of human patients with COVID-19. Four doses of nanodecoy inhalation led to a reduction of viral load in both BAL and NS 8 days following SARS-CoV-2 challenge. Adverse events such as weight loss, fever, or mortality were not observed. Histopathology, immunohistochemistry, RNAscope, and immunofluorescence analyses of lung tissues demonstrated that the nanodecoys was not only effective in alleviating inflammatory cell infiltration and decreasing pulmonary fibrosis but more importantly, was capable of reducing the levels of SARS nucleocapsid protein (SARS-N) and viral RNA. To those ends, results of the present disclosure demonstrate that LSC-nanodecoys can serve as a potent and effective therapeutic agent for treating COVID-19.

Previous studies have indicated that ACE2 is the host receptor for the novel coronavirus (SARS-CoV-2) and that viral entry of SARS-CoV-2 depends on the binding of the viral spike S1 to ACE2 on the host cells. Therefore, inhibiting the binding of spike S1 to ACE2 is a possible treatment strategy to combat COVID-19. Based on this, prior studies have focused on blocking SARS-CoV-2 entry by using recombinant ACE2 (rACE2) protein, such as rACE2 alone or rACE2 fused with an Fc fragment (rACE2-Fc). However, those protein-based neutralization strategies are limited by their overall short half-life after administration. Furthermore, undesired dosage and distribution of extracellular ACE2 could cause unknown toxicity effects on the body. In addition, except for ACE2, other components on cell membranes also play roles in virus docking; therefore, targeting ACE2 alone may not be enough.

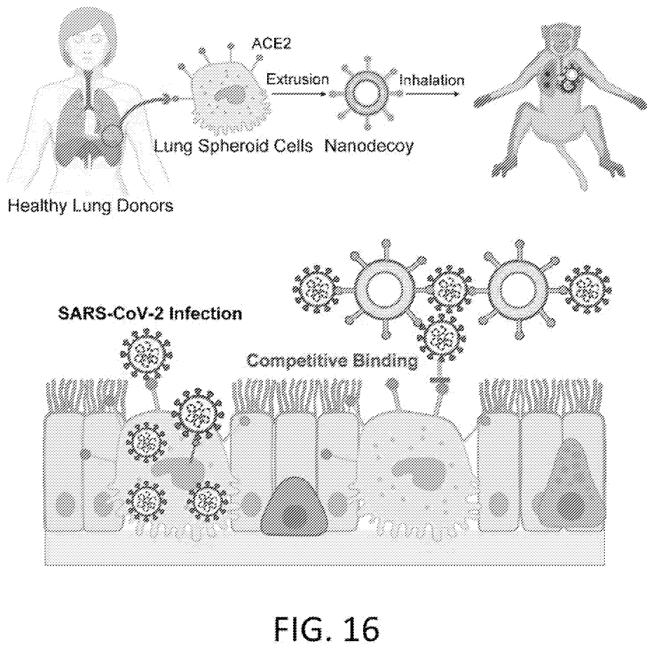

Previous studies have shown several anti-microbial applications by utilizing cell membrane-based nanodecoys. For example, nanodecoys from Aedes albopictus (C6/36) cell membrane-coated gelatin nanoparticles have been developed to trap Zika virus for preventing viral infection. Also, T-cell-membrane-coated nanoparticles were used as decoys for HIV neutralization owing to the presence of T-cell surface antigens for HIV binding. In addition to cellular-membrane-based nanodecoys, engineered liposomes have also been fabricated as decoy targets to sequester bacterial toxins produced during active infection in vivo. As described further herein, embodiments of the present disclosure provide nanodecoy treatment for COVID-19 (FIG. 16). The nanodecoys could be generated from human lung cells in a large scale using commercially available extrusion devices. They not only express natural human ACE2 but also represent a mimic of human lung cells, which are the main targets of SARS-CoV-2.

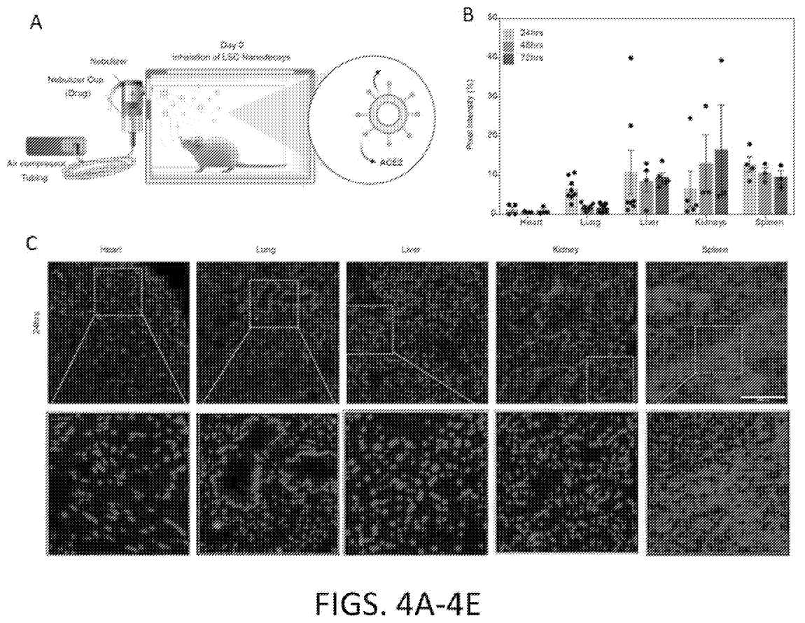

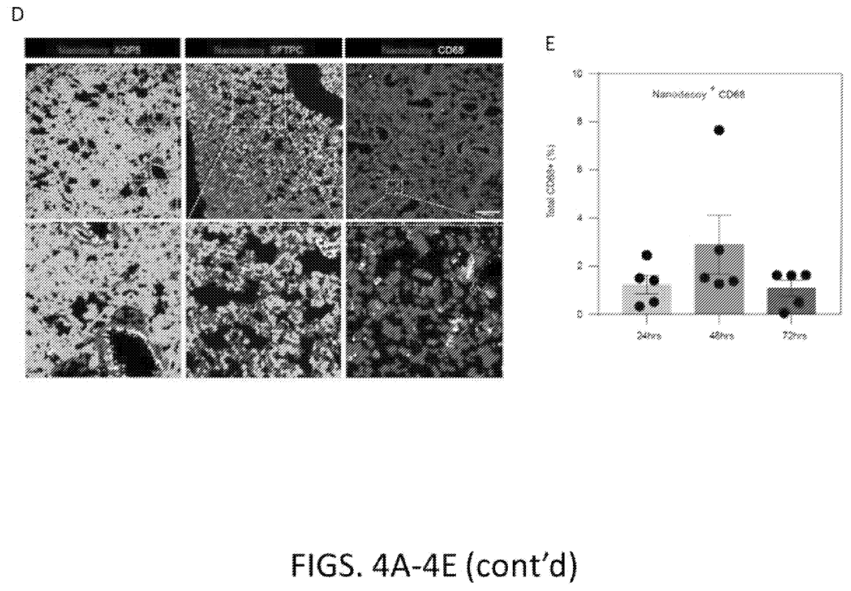

One concern of drug development is the potential off-target effects and undesired biodistribution. Embodiments of the present disclosure provide a simple and clinically relevant method of nanodecoy delivery via inhalation using a nebulizer instead of traditional intravenous (IV) injection (FIG. 4A). Inhalation of nanodecoys resulted in the direct accumulation of the therapeutic particles in the lungs, which is one of the primary sites of SARS-CoV-2 infections and replication. From just one single inhalation treatment, DiD-labeled nanodecoys can still be found in the lungs after 72 hours (FIG. 4B). Nanodecoys were also detected in the liver, kidney, and spleen throughout the 72 hours, which can be attributed to the metabolization of the nanodecoys, potentially by macrophages. Recently, nanotechnological tools have been used for the treatment of COVID-19 and some recent perspectives and research papers hint at the potential of “nanodecoys” or “nanosponges” for treating SARS-CoV-2 with some basic in vitro or in vivo data. However, no previous studies have tested nanodecoys in any animal models of live SARS-CoV-2 infection.

Exosome-Based VLP Vaccine Platform. Coronavirus disease 2019 (COVID-19) has engulfed the world in a pandemic, negatively impacting countries' financial and social systems. First isolated from an infected individual in Wuhan, China on December, 2019, there have since been 106,125,682 confirmed cases and 2,320,497 deaths worldwide reported by the World Health Organization as of Feb. 9, 2021 (covid19.who.int/). There is an urgent need for effective vaccines against the novel severe acute respiratory syndrome coronavirus 2 (SARS-CoV-2). The first two approved vaccines at US are both messenger RNA (mRNA)-based vaccines (manufactured by Pfizer/BioNTech and Moderna). They both require deep-freezing for transportation and long-term storage. Additionally, the administration route is intramuscular injection therefore can only be done by healthcare providers. Those limitations stress the already worn out healthcare system in the pandemic. To circumvent such limitations, embodiments of the present disclosure include a novel vaccine candidate with the following advantages: 1) lyophilizable and stable at room temperature for weeks; 2) available for self-administration at home through inhalation delivery.

SARS-CoV-2 belongs to the coronavirus family which consists of enveloped, positive-stranded RNA viruses that utilize spike protein complexes to recognize and bind to host cell receptors. Specifically, the receptor-binding domain (RBD) in the SARS-CoV and SARS-CoV-2 spike protein S1 subunit binds to the host airway epithelium angiotensin-converting enzyme 2 (ACE2) receptor and then fuses the viral and host membrane through the S2 subunit, making RBD a specific target for neutralizing antibodies and vaccines. Previous studies have demonstrated the efficacy of SARS-CoV RBD as the target of potently neutralizing antibodies. In vitro studies of SARS-CoV-2 show host antibody engagement with the RBD, binding to and exerting a neutralizing effect. It also blocked the entry of SARS-CoV-2 and SARS-CoV into host ACE2 expressing cells, suggesting its potential as a viral attachment inhibitor. However, administration of RBD alone does not allow for specific targeted delivery and does not evade degradation or rapid clearance. The RBD must be protected through a drug delivery platform that optimizes dosage to the antigen presenting cells (APCs).

Virus-like particles (VLPs) and nanoparticles (NPs) are powerful drug delivery carriers capable of enhancing targeted drug delivery. In particular, exosomes are a type of naturally-occurring extracellular vesicles found in the body, making them a native and ideal delivery vesicle for targeted drug delivery. Because they carry and express their parent cell's RNAs, proteins, and lipids, and express parent surface proteins and receptors, they are superior at targeting same tissue-recipient cells. They contain a cocktail of molecular components composed of proteins, lipids, and nucleic acids with therapeutic properties. In addition, exosomes may be engineered by creating surface modifications to express proteins or peptides to enhance targeting.

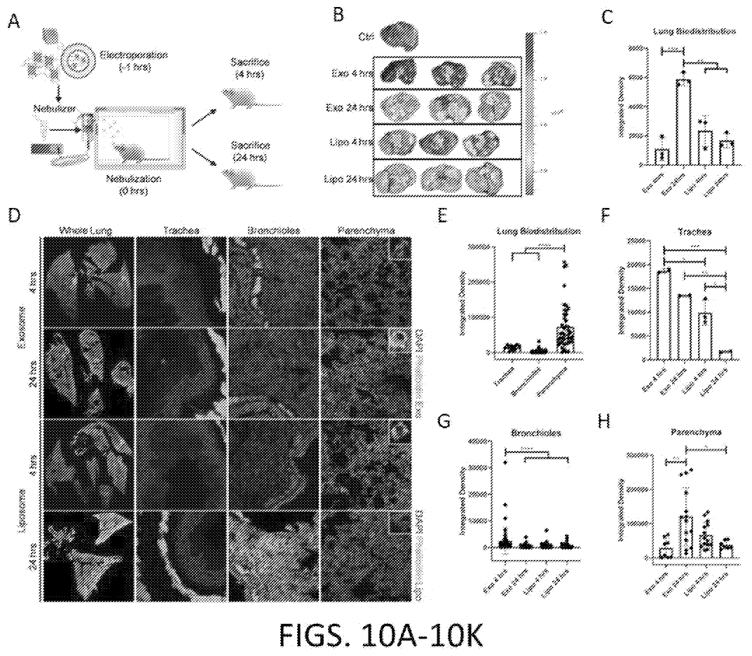

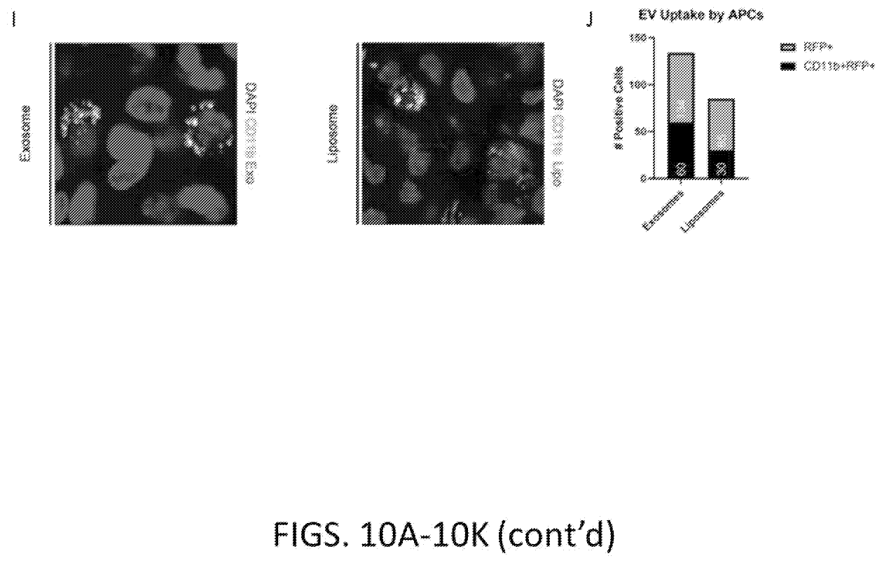

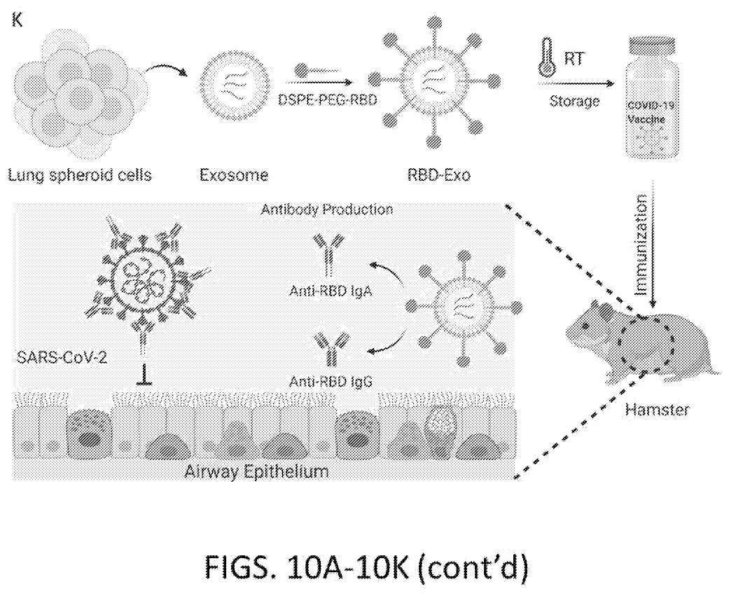

As described further herein, lung spheroid cells (LSCs) were successfully derived from human lung donor samples. Their regenerative abilities have been demonstrated in rodent models and are being tested in human clinical trials (HALT-IPF, www.clinicaltrials.gov). The safety and biodistribution of LSC-derived exosomes (LSC-Exo) were previously studied, for example, through nebulization treatments in rodent models of IPF. LSC-Exo are native NPs for lung therapeutics, derived from heterogeneous populations of lung cells including type I and type II pneumocytes and mesenchymal cells. Previous studies have also confirmed successful exosome delivery throughout the bronchi and parenchyma of the rodent lung. Utilizing the characteristics of LSC-Exo and RBDs, an inhalable vaccine was engineered by conjugating RBD onto the surface of LSC-Exo (RBD-Exo), creating a VLP that emulates the morphology of the native virus. After that, RBD-Exo were delivered via nebulization. In contrast to reported intramuscular COVID-19 vaccines, inhaled RBD-Exo not only induced the production of neutralizing antibodies, but also triggered the mucosal immune system to produce antigen-specific secretory IgA (SIgA). RBD-Exo inhalation is able to suppress viral uptake by the lung epithelium and induce neutralizing antibodies against SARS-CoV-2 (FIG. 10K).

Although novel pharmacological and nanomedicine treatment strategies have been proposed for COVID-19, effective vaccination is still the only way to control and eliminate this pandemic. RBD-based vaccines have shown clinical promise in producing an antibody response capable of protecting against and neutralizing SARS-CoV-2. The airway mucosal immune response plays an integral role in early pathogen invasion, triggering both humoral and cell-mediated immune reactions that trigger systemic responses. To that end, the SARS-CoV-2 RBD was conjugated onto lung-derived exosomes as an inhalable VLP vaccine. Those VLPs trigger robust production of RBD-specific IgG and IgA to neutralize SARS-CoV-2. LSC-Exo is an ideal platform for SARS-CoV-2 VLP. Being native to the lung, LSCs and their derived LSC-Exo share surface proteins and receptors to the membrane features found amongst the airway epithelium. For this reason, exosomes are more distributed and longer retained in the lung and have enhanced internalization by APCs in the lung, providing a more targeted delivery carrier than commonly used liposomes. These data demonstrate that RBD-Exo vaccination led to both humoral and cellular immune responses, protecting against a SARS-CoV-2 mimic in mice and live SARS-CoV-2 infection in a hamster model. Importantly, RBD-Exo vaccination produced high titers of RBD-specific IgGs and IgAs, which play key roles in protecting the lungs against viral invasion in the airway mucosa.

Although most reported vaccines are delivered by intramuscular injection, embodiments of the present disclosure demonstrate that inhalation is an effective administration route when exosomes are used as the vaccine carrier. Without the need to use needles, nebulization of VLPs is highly accessible since it can be performed at home by a single individual and therefore circumvents the need for administration by trained professionals at healthcare facilities. This simplifies the logistics of distribution, lessens the burden of the pandemic on healthcare staff, and greatly reduces exposure to COVID-19. Another challenge is that current vaccine products require storage temperatures as low as −20° C. or −70° C. to ensure stability and preservation. However, maintaining such temperatures in transit is costly and requires specialized containers to control temperature. Upon arrival, vaccines must be stored in deep freezers to maintain efficacy and shelf life, but many consumers, such as hospitals, do not have the proper facility configurations or space to accommodate these freezers, limiting vaccine distribution. On the contrary, RBD-Exo VLPs are stable at room temperature and lyophilizable, extending shelf life, reducing transportation costs, facilitating distribution, and increasing accessibility. These results indicate that this room temperature-stable and inhalable RBD-Exo vaccine represents a promising vaccine candidate to control SARS-CoV-2 infection and the on-going COVID-19 pandemic.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., antiviral treatment). The compositions of the present disclosure may be administered by oral, parenteral (e.g., intramuscular, intraperitoneal, intravenous, ICV, intracistemal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition in the patient (e.g., viral infection). The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

Formulations suitable for nebulizing administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The term “culturing” as used herein refers to growing cells or tissue under controlled conditions suitable for survival, generally outside the body (e.g., ex vivo or in vitro). The term includes “expanding,” “passaging,” “maintaining,” etc. when referring to cell culture of the process of culturing. Culturing cells can result in cell growth, differentiation, and/or division.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “exosomes” as used herein refers to small secreted vesicles (typically about 30 nm to about 250 nm (or largest dimension where the particle is not spheroid)) that may contain, or have present in their membrane or contained within their membrane, nucleic acid(s), protein, small molecule therapeutics, or other biomolecules and may serve as carriers of this cargo between diverse locations in a body or biological system. The term “exosomes” as used herein advantageously refers to extracellular vesicles that can have therapeutic properties, including, but not limited to LSC exosomes.

Exosomes may be isolated from a variety of biological sources including mammals such as mice, rats, guinea pigs, rabbits, dogs, cats, bovine, horses, goats, sheep, primates or humans. Exosomes can be isolated from biological fluids such as serum, plasma, whole blood, urine, saliva, breast milk, tears, sweat, joint fluid, cerebrospinal fluid, semen, vaginal fluid, ascetic fluid and amniotic fluid. Exosomes may also be isolated from experimental samples such as media taken from cultured cells (“conditioned media,” cell media, and cell culture media). Exosomes may also be isolated from tissue samples such as surgical samples, biopsy samples, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes may be isolated from freshly collected samples or from samples that have been stored frozen or refrigerated. Although not necessary, higher purity exosomes may be obtained if fluid samples are clarified before precipitation with a volume-excluding polymer, to remove any debris from the sample. Methods of clarification include centrifugation, ultracentrifugation, filtration or ultrafiltration.

The genetic information within the extracellular vesicle such as an exosome may easily be transmitted by fusing to the membranes of recipient cells, and releasing the genetic information into the cell intracellularly. Though exosomes as a general class of compounds represent great therapeutic potential, the general population of exosomes are a combination of several class of nucleic acids and proteins which have a constellation of biologic effects both advantageous and deleterious.

The term “vesicle” or “nanovesicle” as used herein can refers to a vesicle secreted by cells or derived from cells (e.g., via extrusion process) that may have a larger diameter than that referred to as an “exosome.” Vesicles and nanovesicles (alternatively named “microvesicle” or “membrane vesicle”) may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Typically, at least part of the membrane of the extracellular vesicle is directly obtained from a cell (also known as a donor cell).

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear, in the event, however of any latent ambiguity, definitions provided herein take precedent over any dictionary or extrinsic definition. Further, unless otherwise required by context, singular terms shall include pluralities and plural terms shall include the singular.

### 2. Nanovesicles and Related Compositions

Embodiments of the present disclosure include compositions that include a plurality of nanovesicles having at least one cell surface protein capable of binding a virus. In general, the nanovesicles can be derived from any cell, including but not limited to a lung spheroid cell (LSC), according to the methods described further herein (e.g., extrusion process). As would be recognized by one of ordinary skill in the art based on the present disclosure, nanovesicles derived from cells are not naturally-occurring; however, they may share one or more features of the parent cell from which they were derived.

In some embodiments, a nanovesicle derived from a lung spheroid cell (LSC) includes a cell surface protein that binds or is recognized by an infectious pathogen, such as a virus or bacteria. In one embodiments, the cell surface protein that is recognized by the infectious pathogen is Angiotensin-converting enzyme 2 (ACE2), or a derivative or fragment thereof. In some embodiments, the ACE2 protein or derivative or fragment thereof is endogenous to the cell (e.g., present on the parent cell from which the nanovesicle was derived). In some embodiments, the ACE2 protein or derivative or fragment thereof is exogenous to the cell (e.g., not present on the parent cell from which the nanovesicle was derived). An exogenous cell surface protein includes those which may have been engineered to be expressed in the parent cell or the nanovesicle (e.g., recombinant proteins, peptides, and polypeptides), but which are not generally endogenously present.

In some embodiments, the at least one cell surface protein includes other proteins, peptides, or polypeptides that are markers of the parent cell. In the case of LSCs, the other cell surface proteins can include, but are not limited to, AQP5, SFTPC, CD68, EpCAM, CD90, and/or MUC5b. Such cell surface proteins can be used as biomarkers and/or they can be used to for sorting or purification purposes. In some cases, the cell surface proteins are also recognized by a virus or other pathogenic organism.

The size of the nanovesicles will depend on the methods employed to derive them from a parent cell, as well as other factors, such as how the nanovesicles will be delivered or administered to a subject for therapeutic purposes. In some embodiments, the plurality of nanovesicles comprise an average size ranging from about 50 nm to about 1000 nm. In some embodiments, the plurality of nanovesicles comprise an average size ranging from about 50 nm to about 900 nm. In some embodiments, the plurality of nanovesicles comprise an average size ranging from about 50 nm to about 800 nm. In some embodiments, the plurality of nanovesicles comprise an average size ranging from about 50 nm to about 700 nm. In some embodiments, the plurality of nanovesicles comprise an average size ranging from about 50 nm to about 600 nm. In some embodiments, the plurality of nanovesicles comprise an average size ranging from about 50 nm to about 500 nm. In some embodiments, the plurality of nanovesicles comprise an average size ranging from about 50 nm to about 400 nm. In some embodiments, the plurality of nanovesicles comprise an average size ranging from about 100 nm to about 1000 nm. In some embodiments, the plurality of nanovesicles comprise an average size ranging from about 200 nm to about 1000 nm. In some embodiments, the plurality of nanovesicles comprise an average size ranging from about 300 nm to about 1000 nm. In some embodiments, the plurality of nanovesicles comprise an average size ranging from about 400 nm to about 1000 nm. In some embodiments, the plurality of nanovesicles comprise an average size ranging from about 500 nm to about 1000 nm. In some embodiments, the plurality of nanovesicles comprise an average size ranging from about 600 nm to about 1000 nm. In some embodiments, the plurality of nanovesicles comprise an average size ranging from about 700 nm to about 1000 nm. In some embodiments, the plurality of nanovesicles comprise an average size ranging from about 00 nm to about 1000 nm. In some embodiments, the plurality of nanovesicles comprise an average size ranging from about 00 nm to about 1000 mu. In some embodiments, the plurality of nanovesicles comprise an average size ranging from about 200 nm to about 900 nm. In some embodiments, the plurality of nanovesicles comprise an average size ranging from about 300 nm to about 800 nm. In some embodiments, the plurality of nanovesicles comprise an average size ranging from about 400 nm to about 700 nm. In some embodiments, the plurality of nanovesicles comprise an average size ranging from about 200 nm to about 400 nm. In some embodiments, the plurality of nanovesicles comprise an average size ranging from about 300 nm to about 400 nm. In some embodiments, the plurality of nanovesicles comprise an average size of about 300 nm. In some embodiments, the plurality of nanovesicles comprise an average size of about 310 nm. In some embodiments, the plurality of nanovesicles comprise an average size of about 320 nm. In some embodiments, the plurality of nanovesicles comprise an average size of about 330 nm. In some embodiments, the plurality of nanovesicles comprise an average size of about 340 nm. In some embodiments, the plurality of nanovesicles comprise an average size of about 350 nm.

In some embodiments, the composition further comprises at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the nanovesicles, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered nanovesicle and any corresponding cargo (e.g., vaccine). Adjusting the dose to achieve maximal efficacy based on the methods described above and other methods as are well-known in the art is well within the capabilities of the ordinarily skilled person in the art.

In some embodiments, the plurality of nanovesicles include cell surface proteins capable of binding a virus, such as a coronavirus. In some embodiments, the coronavirus is selected from the group consisting of 229E, NL63, OC43, HKU1, MERS-CoV, SARS-CoV, and SARS-CoV-2. Coronaviruses are a family of enveloped RNA viruses (positive-strand RNA viruses) that are distributed widely among mammals and birds, causing principally respiratory or enteric diseases but in some cases neurologic illness or hepatitis. Individual coronaviruses usually infect their hosts in a species-specific manner, and infections can be acute or persistent. Infections are transmitted mainly via respiratory and fecal-oral routes. The most distinctive feature of this viral family is genome size: coronaviruses have the largest genomes among all RNA viruses, including those RNA viruses with segmented genomes. This expansive coding capacity seems to both provide and necessitate a wealth of gene-expression strategies.

In some embodiments, the nanovesicles of the present disclosure include intra-vesicle cargo. In some embodiments, the plurality of nanovesicles can have cargo that includes at least one therapeutic protein, peptide, polypeptide, nucleic acid molecule, polynucleotide, mRNA, siRNA, miRNA, antisense oligonucleotide, drug, or therapeutic small molecule. In some embodiments, the cargo can enhance binding to a virus and/or enhance a therapeutic effect that the nanovesicle exerts against a virus.

Embodiments of the present disclosure also includes methods of generating a plurality of nanovesicles for the treatment and/or prevention of a viral infection. In accordance with these embodiments, the methods include culturing a plurality of parental cells from which the nanovesicles are derived, such as lung spheroid cells (LSCs). Parental cells can be cultured in 2D or 3D cell culture platforms. In some embodiments, the method includes subjecting the plurality of parental cells to an extrusion process to produce the plurality of nanovesicles having the desired characteristics. In some embodiments, the extrusion process comprises passing the parental cells (e.g., LSCs) through an extruder comprising at least one of a 5 μm, a 1 μm, and/or a 400 nm pore-sized membrane filters. As would be recognized by one of ordinary skill in the art, other filter sizes and combinations can be used in the extrusion process, depending on the nanovesicle size and characteristics desired.

In some embodiments, the method further includes purifying and concentrating the plurality of nanovesicles using ultrafiltration or other filtration means known in the art. In some embodiments, the nanovesicles can be selected, sorted, purified, or concentrated based on the use of one or more cell surface proteins.

Embodiments of the present disclosure also include compositions that include a plurality of exosomes derived from a cell. In general, the exosomes can be derived from any cell, including but not limited to a lung spheroid cell (LSC), according to the methods described further herein, as well as those methods described in PCT/US2019/039721, which is herein incorporated by reference in its entirety. As would be recognized by one of ordinary skill in the art based on the present disclosure, exosomes derived from cells are not naturally-occurring; however, they may share one or more features of the parent cell from which they were derived.

In accordance with these embodiments, the compositions of the present disclosure include a plurality of exosomes comprising at least one membrane-associated protein on the surface of the plurality of exosomes (e.g., a cell surface receptor or binding protein). In some embodiments, the membrane-associated protein on the surface of the plurality of exosomes is a viral-specific protein, such as a viral protein, peptide, or polypeptide that can induce an immunogenic response in a subject (e.g., a viral antigen or epitope). In some embodiments, the viral-specific protein on the surface of the exosomes comprises a Spike protein (S protein), or fragment or derivative thereof, of a coronavirus (e.g., 229E, NL63, OC43, HKU1, MERS-CoV, SARS-CoV, and SARS-CoV-2). As would be recognized by one of ordinary skill in the art based on the present disclosure the plurality of exosomes can be generated to include any other membrane-associated proteins on their surfaces capable of generating an immunogenic response in a subject as part of a vaccine composition.

In some embodiments, the plurality of exosomes can be generated to include one or more therapeutic agents contained within their membranes (e.g., cargo), which can further enhance an immune response in a subject. Such therapeutic agents can include any protein, peptide, polypeptide, nucleic acid, small molecule compound, or any combinations or derivatives thereof that can enhance an immune response in a subject. In some embodiments, the therapeutic agent is an mRNA or fragment thereof that can be a basis for producing more viral antigens or antigenic epitopes to stimulate a subject's immune system as part of a vaccine composition. In some embodiments, the mRNA can encode a viral antigen or antigenic epitope that is the same or different from the membrane-associated protein on the surface of the plurality of exosomes described above. In some embodiments, the mRNA can encode a Spike protein (S protein), or fragment or derivative thereof, of a coronavirus (e.g., 229E, NL63, OC43, HKU1, MERS-CoV, SARS-CoV, and SARS-CoV-2).

In some embodiments, the membrane-associated protein on the surface of the plurality of the exosomes comprises a protein capable of binding a virus. In accordance with these embodiments, the exosomes can function as a drug delivery platform for targeting a virus with one or more antiviral therapeutics. For example, in some embodiments, the protein capable of binding a virus comprises Angiotensin-converting enzyme 2 (ACE2), or a derivative or fragment thereof, which has been identified as a binding site for SARS-CoV-2 (COVID-19). As would be recognized by one of ordinary skill in the art based on the present disclosure, the plurality of exosomes can be generated to include any other membrane-associated proteins on their surfaces capable of binding to a virus as part of a therapeutic antiviral drug delivery platform.

In accordance with these embodiments, the exosomes can be generated to include one or more antiviral therapeutic agents, such as agents that reduce viral load by targeting the ability of the virus to infect or reproduce within a subject. Antiviral therapeutic agents can include, but are not limited to, SARS-CoV-2 (COVID-19) antiviral agents such as remdesivir, interferon beta-1b, and/or lopinavir-ritonavir, among others. In some embodiments, the antiviral therapeutic agent is remdesivir, interferon beta-1b, and/or lopinavir-ritonavir, and it is contained within a plurality of LSC exosomes to target SARS-CoV-2 (COVID-19).

In some embodiments, the exosome compositions described herein further comprise at least one pharmaceutically acceptable excipient or carrier. Embodiments of the present disclosure include a pharmaceutical composition comprising a plurality of LSC-derived exosomes in an amount effective in modulating a pulmonary pathological condition when delivered to an animal or human subject in need thereof. In some embodiments of the present disclosure, the pulmonary pathological condition is a viral infection, such as a coronavirus infection (e.g., COVID-19).

In some embodiments of the present disclosure, the pharmaceutical composition can comprise at least one of an isolated LCS exosome comprising on its membrane surface or contained within it a polypeptide, a peptide, a nucleic acid, or a small molecule therapeutic. In some embodiments of the present disclosure, the pharmaceutical composition can comprise a population of lung spheroid cell-derived exosomes isolated from a lung spheroid cell-conditioned medium. In some embodiments of the present disclosure, the nucleic acid can be an miRNA (e.g., an mRNA encoding an immunogenic viral epitope). In some embodiments of the present disclosure, the pharmaceutical composition administered to the respiratory tract of the animal or human subject can further comprise a pharmaceutically acceptable carrier. In accordance with these embodiments, the pharmaceutical composition an immune response in a subject. For example, the composition can induce a mucosal and systemic immune response against the exogenous polypeptide. In some embodiments, the composition increases immunoglobulin A (IgA) antibodies specific for a viral antigen. In some embodiments, the composition increases immunoglobulin G (IgG) antibodies specific for a viral antigen. In some embodiments, the pharmaceutical composition induces an immune response in a subject such that sufficient antibodies are produced to neutralize viral load.

In some embodiments, the vaccine compositions of the present disclosure are stable at room temperature (e.g., 15-25° C.). In some embodiments, the vaccine compositions of the present disclosure are stable below room temperature. In some embodiments, the vaccine compositions of the present disclosure are stable above room temperature. In some embodiments, the vaccine compositions of the present disclosure are stable at room temperature for at least 6 hours. In some embodiments, the vaccine compositions of the present disclosure are stable at room temperature for up to an including 6 months. In some embodiments, the vaccine compositions of the present disclosure are stable at room temperature from about 1 day to about 6 months, from about 1 day to about 5 months, from about 1 day to about 4 months, from about 1 day to about 3 months, from about 1 day to about 2 months, from about 1 day to about 1 month, from about 1 day to about 4 weeks, from about 1 day to about 3 weeks, from about 1 day to about 2 weeks, and from about 1 day to about 1 week.

A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the nanovesicles, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered nanovesicle and any corresponding cargo (e.g., vaccine). Adjusting the dose to achieve maximal efficacy based on the methods described above and other methods as are well-known in the art is well within the capabilities of the ordinarily skilled person in the art.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The vaccine compositions described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, inhalable, topical, injectable, immediate-release, pulsatile-release, delayed-release, or sustained release). The vaccine compositions may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### 3. Therapeutic Methods

Embodiments of the present disclosure also include a method of treating a viral infection comprising administering any of the compositions described above to a subject in need thereof. In some embodiments, the composition is administered orally, parenterally, intramuscularly, intraperitoneally, intravenously, intracerebroventricularly, intracisternally, subcutaneously, via injection or infusion, via inhalation, spray, nasal, vaginal, rectal, sublingual, or topical administration. In some embodiments, the composition is administered via nebulization to lung tissue.

In some embodiments, administration of the plurality of nanovesicles or exosomes reduces viral load in the subject. As would be recognized by one of ordinary skill in the art based on the present disclosure, pharmaceutical compositions comprising a plurality of nanovesicles or exosomes can be administered in an amount effective such that neutralization of a virus (e.g., SARS-CoV-2) is achieved. In some embodiments, the composition is administered (e.g., via inhalation) at a dose of about 1×10⁷ to about 1×10¹³ particles per kg of body weight. In some embodiments, the composition is administered at a dose of about 1×10⁸ to about 1×10¹² particles per kg of body weight. In some embodiments, the composition is administered at a dose of about 1×10⁹ to about 1×10¹¹ particles per kg of body weight. In some embodiments, the composition is administered at a dose of about 1×10⁷ particles per kg of body weight, about 1×10⁸ particles per kg of body weight, about 1×10⁹ particles per kg of body weight, about 1×10¹⁰ particles per kg of body weight, about 1×10¹¹ particles per kg of body weight, about 1×10¹² particles per kg of body weight, about 1×10¹³ particles per kg of body weight, about 1×10¹⁴ particles per kg of body weight, or about 1×10¹⁵ particles per kg of body weight.

In accordance with these embodiments, the plurality of nanovesicles or exosomes of the present disclosure can persist in the subject's tissues (e.g., lung tissue) for at least 72 hours after administration. In some embodiments, the plurality of nanovesicles or exosomes persist in a subject for at least 12 hours, at least 24 hours, at least 36 hours, at least 48 hours, at least 60 hours, at least 72 hours, at least 84 hours, and at least 96 hours. In some embodiments, the plurality of nanovesicles or exosomes are administered every 24 hours, every 48 hours, every 72 hours, or every 96 hours, depending on the dose being administered and the subject's physiological characteristics.

In some embodiments, a single dose of the plurality of nanovesicles or exosomes of the present disclosure can exert a beneficial effect (e.g., promote viral clearance, reduce tissue damage, reduce viral infection rate, and the like) on a subject. In some embodiments, two or more doses are required to provide a beneficial effect. In some embodiments, three or more doses are required to provide a beneficial effect. In some embodiments, four or more doses are required to provide a beneficial effect. In some embodiments, five or more doses are required to provide a beneficial effect. In some embodiments, six or more doses are required to provide a beneficial effect. In some embodiments, seven or more doses are required to provide a beneficial effect. In some embodiments, eight or more doses are required to provide a beneficial effect. In some embodiments, nine or more doses are required to provide a beneficial effect. In some embodiments, ten or more doses are required to provide a beneficial effect.

In some embodiments, the nanovesicles (e.g., nanodecoys) and the LSC exosomes can be used to treat and/or prevent a viral infection. In some embodiments, the viral infection is caused by a coronavirus (e.g., 229E, NL63, OC43, HKU1, MERS-CoV, SARS-CoV, and SARS-CoV-2). In some embodiments, the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration. In some embodiments, the composition is administered via nebulization to lung tissue. In some embodiments, the virus is a coronavirus. In some embodiments, the coronavirus is selected from the group consisting of 229E, NL63, OC43, HKU1, MERS-CoV, SARS-CoV, and SARS-CoV-2.

One of the important advantages of the embodiments of the present disclosure includes the use of LSC-derived exosomes as a vaccine delivery platform. That is, this alternative strategy to parenteral vaccination includes targeting SARS-CoV-2 at the points of transmission and replication: the respiratory and intestinal mucosa. A vaccine strategy that protects the mucosa and the associated initial cellular targets can be critical for protection against SARS-CoV-2 infection and replication. The mucosal immune system is, in many respects, independent of the systemic immune system. For example, 90% of mucosal IgA is produced locally and induction of mucosal immunity is best achieved via mucosal vaccination. While the focus of vaccine testing is often on the induction of neutralizing antibodies, IgA has been shown to protect against viral infections with a broader array of effector functions that include immune exclusion, pathogen aggregation, intracellular neutralization, virus excretion (reverse transcytosis), as well as classical neutralization. As described further herein, inhaled RBD-Exo vaccine not only induced the production of neutralizing antibodies, but also triggered the mucosal immune system to produce antigen-specific secretory IgA (SIgA). RBD-Exo inhalation suppressed viral uptake by the lung epithelium and induce neutralizing antibodies against SARS-CoV-2.

As would be recognized by one of ordinary skill in the art based on the present disclosure, neutralizing antibodies induced by the vaccine compositions described herein can bind to any known or as yet undiscovered coronavirus, such as, for example, coronavirus OC43, coronavirus 229E, coronavirus NL63, coronavirus HKU1, MERS-CoV, SARS-CoV, or SARS-CoV-2 (COVID-19). In some embodiments, neutralizing antibodies generated by the vaccine compositions of the present disclosure are directed against SARS-CoV-2 (COVID-19). In the context of the present disclosure a “neutralizing antibody” can include an antibody that binds to a virus (e.g., a coronavirus) and interferes with the virus' ability to infect a host cell. Coronavirus spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The S1 subunit of the ectodomain mediates binding of the virion to host cell-surface receptors through its receptor-binding domain (RBD). The S2 subunit fuses with both host and viral membranes, by undergoing structural changes.

SARS-CoV-2 utilizes the Spike glycoprotein to interact with cellular receptor ACE2 (Zhou et al., Nature 579: 270-273, doi:10.1038/s41586-020-2012-7 (2020); Hoffmann et al., Cell, S0092-8674(0020)30229-30224, doi:10.1016/j.cell.2020.02.052 (2020) doi:10.1016/j.cell.2020.02.052 (2020). The amino acid sequence of the SARS-CoV-2 spike protein has been deposited with the National Center for Biotechnology Information (NCBI) under Accession No. QHD43416. Binding with ACE2 triggers a cascade of cell membrane fusion events for viral entry. The high-resolution structure of SARS-CoV-2 RBD bound to the N-terminal peptidase domain of ACE2 has recently been determined, and the overall ACE2-binding mechanism is virtually the same between SARS-CoV-2 and SARS-CoV RBDs, indicating convergent ACE2-binding evolution between these two viruses (Gui et al., CellRes 27, 119-129, doi:10.1038/cr.2016.152 (2017); Song et al., PLoS Pathog 14, e1007236-e1007236, doi:10.1371/journal.ppat.1007236 (2018); Yuan et al., Nat Commun 8, 15092-15092, doi:10.1038/ncomms15092 (2017); and Wan et al., J Virol, JVI.00127-00120, doi:10.1128/JVI.00127-20 (2020)). This suggests that disruption of the RBD and ACE2 interaction, e.g., by neutralizing antibodies, would block SARS-CoV-2 entry into the target cell. The peptide comprising a receptor binding domain (RBD) of a coronavirus spike protein may be prepared using routine molecular biology techniques, such as those disclosed herein. The nucleic acid and amino acid sequences of RBDs of various coronavirus spike proteins are known in the art (see, e.g., Tai et al., Cell Mol Immunol 17, 613-620 (2020). doi.org/10.1038/s41423-020-0400-4; and Chakraborti et al., Virology Journal volume 2, Article number: 73 (2005); and Chen et al., Biochemical and Biophysical Research Communications, 525(1): 135-140 (2020)).

In another aspect, embodiments of the present disclosure encompass methods of treating a pathological condition of an animal or human pulmonary system, wherein the method comprises administering to a region of the respiratory tract of an animal or human subject a pharmaceutical composition comprising a plurality of lung spheroid cell-derived exosomes in an amount effective in modulating a pulmonary pathological condition when delivered to the animal or human subject in need thereof. In some embodiments of this aspect of the disclosure, the pulmonary pathological condition is a viral infection, such as a coronavirus infection (e.g., COVID-19). In some embodiments of this aspect of the disclosure, the pharmaceutical composition can comprise a population of lung spheroid cell-derived exosomes isolated from a lung spheroid cell-conditioned medium.

The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein.

A subject may be treated with a composition of the disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A composition or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously. A beneficial pharmacokinetic profile can be obtained by the administration of a formulation or dosage form suitable for once, twice, or three times a day administration in an amount sufficient to provide a required dose of the composition. Certain dosage forms and formulations may minimize the variation between peak and trough plasma and/or brain levels of compositions of the disclosure and in particular provide a sustained therapeutically effective amount of the compositions. The present disclosure also contemplates a formulation or dosage form comprising amounts of one or more composition of the disclosure that results in therapeutically effective amounts of the composition over a dosing period, in particular a 24 h dosing period. A medicament or treatment of the disclosure may comprise a unit dosage of at least one composition of the disclosure to provide therapeutic effects. A “unit dosage or “dosage unit” refers to a unitary (e.g., a single dose), which is capable of being administered to a subject, and which may be readily handled and packed, remaining as a physically and chemically stable unit dose comprising either the active agents as such or a mixture with one or more solid or liquid pharmaceutical excipients, carriers, or vehicles.

### 4. Materials and Methods

ACE2 Nanodecoys

Coronavirus disease 2019 (COVID-19), caused by severe acute respiratory syndrome coronavirus 2 (SARS-CoV-2), has grown into a global pandemic, and no specific antiviral treatments have been approved to date. The angiotensin-converting enzyme 2 (ACE2) plays a fundamental role in SARS-CoV-2 pathogenesis as it allows viral entry into host cells. Embodiments of the present disclosure demonstrate that ACE2 nanodecoys derived from human lung spheroid cells (LSCs) can bind and neutralize SARS-CoV-2 and protect the host lung cells from infection. In mice, for example, the nanodecoys were delivered via inhalation therapy and resided in the lungs for over 72 hours post-delivery. Furthermore, inhalation of nanodecoys accelerated clearance of SARS-CoV-2 mimics from the lungs, with no observed toxicity. In cynomolgus macaques challenged with live SARS-CoV-2, four doses of nanodecoys delivered by inhalation promoted viral clearance and reduced lung injury. The results described herein indicate that LSC-nanodecoys can serve as a potential therapeutic agent for treating COVID-19.

Generation of nanodecoys. Nanodecoys were derived from LSCs or HEK293 cells (ATCC@ CRL-1573™) by an extruder (AVESTIN LIPOSOFAST LF-50, AVESTIN, Inc). Cells were collected and suspended in PBS at a concentration of 5×10⁶ cells/mL. A large volume of cells could be extruded immediately or stored at −80° C. until ready. The cells were passed through the extruder twice through 5 μm, 1 μm, and 400 nm pore-sized polycarbonate membrane filters (Avanti Polar Lipids, Inc.) sequentially. The resulting nanodecoys were purified and concentrated using an ultrafiltration centrifuge tube (100 kDa MWCO; Millipore) and centrifuged at 4,500 g for 10 min and washed with PBS. The size and concentration of nanodecoys were measured using Nanoparticle Tracking Analysis system (Nanosight, Malvern). Nanodecoys were stored at 4° C. for one week or placed in long-term storage at −80° C. The ACE2 receptors on the nanodecoys were detected using immunoblot, immunostaining, flow cytometry, and transmission electron microscopy (TEM) with immunogold labeling.

ACE2 analysis using ELISA. Approximately 5×10⁶ of LSC and HEK293 cells were collected and 10¹⁰ of LSC-nanodecoy and HEK293-nanodecoy were prepared. They were analyzed with an ACE2 ELISA kit (Abcam, ab235649) according to the manufacturer's instructions.

In vitro Internalization experiments of nanodecoys. Human macrophage primary cells and LSCs (10⁴ cells/mL) were seeded in 4-well culture chamber slides (Thermo Fisher Scientific). Nanodecoys (1×10⁶ cells/mL) were then labeled by DiD and incubated with macrophages or LSCs alone, as well as a co-culture of both (1:1) to mimic the in vivo microenvironment. After 4 hours of incubation, free nanodecoys were removed by 3 washes with 1×PBS. Cells were fixed using 4% PFA prior to immunocytochemistry staining with makers for macrophage (CD4; 12-0041-82, Invitrogen) and LSC (CD90; 11-0909-42, Invitrogen) and imaged with an Olympus FLUOVIEW confocal microscope. In addition, to quantify the internalization rate of nanodecoys by the different cell types, cells and nanodecoys were cultured in a T75 flask as previously described and collected for flow cytometry analysis (CytoFlex; Beckman Coulter).

In vitro spike S1 neutralization experiments of nanodecoys. Recombinant spike S1 (Sino Biological 40591-V08H, 10 ng/mL, MW=76.5 kDa) was added to nanodecoys at different concentrations (5×10⁹, 1×10⁹, 2×10⁸, 4×10⁷, 8×10⁶, 1.6×10⁶, and 3.2×10⁵) and incubated for three hours. After that, the unbound spike S1 was removed by ultracentrifugation (100 kDa). Spike S1 before and after binding to nanodecoys was determined using an ELISA kit (Sino Biological SARS-CoV-2 SPIKE ELISA KIT, Sino Biological) according to manufacturer's protocol. To study the neutralization of spike S1 with nanodecoys in primary lung derived cells (LSCs), spike S1 was first labeled using NHS-Rhodamine (46406, Thermo Fisher Scientific) according to the manufacturer's instructions. The RhB-spike S1 (100 ng) was first incubated with LSCs (2×10⁴) in 4-well slides for 1 h and washed with PBS for three times. After that, DiD labeled nanodecoys (2×10⁷) were added and incubated for another 4 h. Cells were washed and fixed using 4% PFA prior to stain with Alexa Fluor™ 488 Phalloidin (Invitrogen™ A12379). Cells were imaged using an Olympus FLUOVIEW confocal microscope.

Generation of SARS-CoV-2 mimicking virus. Spike S1 (40591-V08H; Sino Biological) was conjugated to lentivirus (Cellomics Technology LLC) to create a SARS-CoV-2 mimic. His-tagged spike S1 was linked to Ni nitrilotriacetate (Ni-NTA) through the chemical interaction. NTA with mercapto group (N-[Nα,Nα-Bis(carboxymethyl)-L-lysine]-12-mercaptododecanamide) was first reacted with 4-(N-Maleimidomethyl)cyclohexane-1-carboxylic acid 3-sulfo-N-hydroxysuccinimide ester sodium salt (Sulfo-SMCC) to give NTA-SMCC and then was added to the lentivirus. The NTA groups were conjugated to the lentivirus through the —NH₂ group on lentivirus and N-hydroxysuccinimide ester on NTA-SMCC. The free NTA-SMCC was removed by centrifugation using an ultrafiltration tube (100 kDa MWCO; Millipore) to give SARS-CoV-2 mimicking virus (spike S1-lentivirus). The successfully conjugated spike S1 on lentivirus was confirmed using TEM. Briefly, SARS-CoV-2 mimics were incubated with anti-Spike S1 antibodies overnight at 4° C. Free antibodies were removed using an ultrafiltration tube (100 kDa MWCO; Millipore) and washed with PBS three times. Spike S1 on the SARS-CoV-2 mimics was labeled with immunogold (10 nm) antibodies and negatively stained for TEM visualization. The conjugation efficiency of spike S1 on lentivirus was determined using ELISA (Sino Biological SARS-COV-2 SPIKE ELISA KIT, Sino Biological) according to manufacturer's protocol.

SARS-CoV-2 mimicking virus in cells. LSC cells (10⁴ cells/mL) were seeded in 8-well culture chamber slides (Thermo Fisher Scientific) and allowed to adhere for 24 hrs. SARS-CoV-2 mimics (10⁴ TU/mL) were added into the 8-well slides and incubated for 4 hrs. After that, LSC cells were washed with PBS twice to remove non-internalized SARS-CoV-2 mimics and stained with 100 μM Lyso Dye (Invitrogen, green) at 37° C. for 30 min. Subsequently, slides were mounted with ProLong Gold Antifade Mountant with 4,6-diamidino-2-phenylindole (DAPI, Invitrogen, Waltham, Mass., USA) and imaged on the Olympus FLUOVIEW CLSM (Olympus; FV3000, Shinjuku, Tokyo, Japan) with an Olympus UPlanSAPO 60× objective (Olympus; 1-U2B832, Shinjuku, Tokyo, Japan).

SARS-CoV-2 mimic neutralization experiment. Nanodecoys were first labeled using DiI. After that, 200 μL of SARS-CoV-2 mimic (5×10⁵) in pH 9.6 coating buffer was added to each well of 96-well plates and incubated at 4° C. overnight for coating. In addition, lentiviruses without spike S1 were also coated to the plates as a control. Following the incubation, the protein solution was removed, and the wells were washed with 1×PBS. To study binding, plates were incubated with DiI-labeled nanodecoys at concentrations of 1×10⁴, 2×10⁴, 4×10⁴, 8×10⁴, 1.6×10⁵, 3.2×10⁵, 6.4×10⁵, 1.28×10⁶ for two hours at room temperature. After that, the plates were rinsed with 1×PBS for three times, and fluorescent intensities were determined using a microplate reader (Molecular Devices).

Interaction of SARS-CoV-2 mimic with LSCs was assessed by ICC and flow cytometry. RhB-NHS was first reactivated with NTA-tagged lentivirus and then modified with S1 protein to synthesize RhB-labeled SARS-CoV-2 mimics. LSCs (10⁴ cells/mL) were seeded in 4-well culture chamber slides. RhB-labeled Lentivirus (10⁴ TU/mL), RhB-labeled SARS-CoV-2 mimic (10⁴ TU/mL), RhB-labeled SARS-CoV-2 mimic (10⁴ TU/mL)+LSC nanodecoys (10⁵), RhB-labeled SARS-CoV-2 mimic (10⁴ TU/mL)+HEK nanodecoys (10⁵) were incubated with LSCs, respectively. After four hours of incubation, free SARS-CoV-2 mimics were removed and washed using PBS for three times. Cells were fixed with 4% PFA, stained for LSC markers (FITC-CD90; 11-0909-42, Invitrogen), and imaged with an Olympus FLUOVIEW confocal microscope. The internalization of SARS-CoV-2 mimics by cells was examined by flow cytometry analysis (CytoFlex; Beckman Coulter).

Nanodecoys protect lung cells from SARS-CoV-2 mimicking viruses. Experiments were conducted to test whether nanodecoys could neutralize SARS-CoV-2 mimic viruses and shelter lung cells from being infected. Macrophages and LSCs (1:1) were co-cultured in 4-well culture chamber slides, and RhB-labeled lentivirus-spike (10⁴ TU/mL) and DiD-labeled nanodecoys (10⁵) were added. After two hours of incubation, free RhB-labeled lentivirus-spike and DiD-labeled nanodecoys were removed and the samples were washed using PBS three times. Cells were fixed with 4% PFA, stained with LSC (FITC-CD90; 11-0909-42, Invitrogen) or macrophages (CD4) markers, and imaged with an Olympus FLUOVIEW confocal microscope. Flow cytometry analysis was performed to confirm the microscopy data.

Biodistribution of nanodecoys in mice. All animal procedures were approved by the Institute Animal Care and Use Committee (IACUC) of North Carolina State University (protocol #19-806-B). Male CD1 mice (7 weeks) were obtained from Charles River Laboratory (Massachusetts, USA). DiD-labeled nanodecoys (1×10¹⁰ particles per kg of body weight) were delivered to the CD1 mice via inhalation treatment using a nebulizer (Pari Trek S Portable Compressor Nebulizer Aerosol System; 047F45-LCS). Mice were euthanized at 24, 48, and 72 hours. All major organs were collected and were cryo-sectioned for further immunofluorescence analysis of the nanodecoys in vivo biodistribution post-inhalation.

In vivo clearance of the SARS-CoV-2 mimicking virus by nanodecoys in mice, Prior to performing clearance assay, the levels of ACE2 on the nanodecoys were quantified by an ELISA analysis (ab235649, Abcam) and was determined to be 112 ACE2 per nanodecoy. AF647-labeled SARS-CoV-2 mimics (5×10⁶ per kg of body weight) were first delivered to the Male CD1 mice (7 weeks) via inhalation treatment using a nebulizer (Pari Trek S Portable Compressor Nebulizer Aerosol System; 047F45-LCS). 24 hours later, nanodecoys (1×10¹⁰ particles per kg of body weight) or free rACE2 with the same amount of ACE2 on the nanodecoys were inhaled, respectively. PBS treatment was used as control. Lungs were collected and imaged 1, 2, 3, 4, 5, and 6 days after treatment using Xenogen Live Imager (IVIS). Additionally, lung tissues were cryo-sectioned for further analysis of SARS-CoV-2 mimics biodistribution in vivo post-inhalation. Blood samples were collected for cytokine array analysis (Mouse Cytokine Array C1000, Raybiotech) according to the manufacturer's instructions.

Toxicity studies in mice. Male CD1 mice (7 weeks) were treated with PBS, LSC- or HEK-nanodecoys (1×10¹⁰ particles per kg of body weight) via inhalation. After 14-day treatment, the blood (blood test) and major organs (H&E) were collected for toxicity evaluation.

Nonhuman primate studies. All animal studies were conducted in compliance with all relevant local, state, and federal regulations and were approved by the Bioqual Institutional Animal Care and Use Committee (IACUC) under approved IACUC #20-090P. Six Cynomolgus Macaques (three females, three males) were allocated by a counterbalance randomization. All animals were housed at Bioqual, Inc. (Rockville, Md.). The macaques were challenged with SARS-CoV-2 using the intranasal and intratracheal routes. The viral inoculum (0.5 mL) will be administered drop-wise into each nostril and 1.0 mL of viral inoculum will be delivered intratracheally using a French rubber catheter/feeding tube, size 10, sterile (cut 4″-6″ in length). Macaques were inoculated with a total dose of 1.1×10⁵ PFU SARS-CoV-2. PBS or the LSC-nanodecoys were administered by inhalation using a nebulization and fitted mask daily from days 2-5 following challenge. Bronchoalveolar lavage (BAL), nasal swabs (NS), blood, body weight, and body temperature were monitored or collected throughout the present disclosure. Macaques were necropsied on day 8 post-challenge. All immunologic and virologic assays were performed blinded.

Statistics and Reproducibility. All experiments were performed at least three times independently. Results are shown as means t SD. Comparisons between any two groups were performed using the two-tailed, unpaired Student's t-test. For multiple group comparison, one-way ANOVA and two-way ANOVA was used with Bonferroni post-correction. A P value less than 0.05 was considered statistically significant.

Cell culture. Human macrophage primary cells (CELPROGEN) were purchased and cultured in pre-coated flasks with human macrophage primary cell culture complete extracellular matrix (Cat #E36070-01) and media with serum (Cat #M36070-01S). Human lung spheroid cells (LSCs) and explant derived cells (EDCs) were generated from healthy whole lung donors acquired from the Cystic Fibrosis and Pulmonary Diseases Research and Treatment Center at the University of North Carolina at Chapel Hill and expanded as previously described. Human lung tissue collection and use are approved by the IRB at the University of North Carolina at Chapel Hill and informed consent was obtained from all subjects prior to tissue collection. All procedures and experiments performed in the present disclosure involving human samples were in accordance with the ethical standards of the IRB and with the guidelines set by the Declaration of Helsinki. Human lung fibroblast cells (ATCC® PCS-201-013™) were obtained from ATCC. All procedures performed in the present disclosure involving human samples were in accordance with the ethical standards of the institutional research committee and with the guidelines set by the Declaration of Helsinki.

Immunoblotting and immunostaining. LSC and EDC cell lysates were analyzed by western blot for ACE2 (MA5-31394; Invitrogen and PA5-85139; Invitrogen) and beta-actin (MA5-15739; Invitrogen) at a 1:1000 dilution and followed by a one-hour incubation with the corresponding HRP conjugated secondary antibodies at a 1:10,000 dilution. Blots were visualized on a Bio-Rad ChemiDoc. Immunostaining was performed on cells or cryo-sectioned tissue slides fixed in 4% paraformaldehyde (PFA), which were permeabilized and blocked with Dako Protein blocking solution (DAKO; X0909) containing 0.1% saponin (47036; Sigma-Aldrich). Cells and tissues were stained with antibodies against ACE2 (MA5-31394; Invitrogen and PA5-85139; Invitrogen), SFTPC (ab3786; Abcam), Phalloidin (ab176753; Abcam), CD4 (12-0041-82, Invitrogen), CD90 (11-0909-42, Invitrogen), and CD68 (ab955; Abcam) at a dilution of 1:100-1:200. Slides were imaged on the Olympus FLUOVIEW confocal microscope and analyzed on ImageJ (imagej.nih.gov/ij/).

Flow cytometry. Cells were washed with MACS flow buffer (130-091-222; MACS) and permeabilized with BD Cytofix/Cytoperm (554714; BD) prior to incubation with antibodies against ACE2 (PA5-85139; Invitrogen), EpCAM (ab71916; Abcam), CD90 (555595; BD), MUC5b (ab77995; Abcam), and vWF (ab11713; Abcam). Nanodecoys were prepared by binding the particles to 4 μm aldehyde/sulfate latex beads (A37304; Thermo Fisher) at 4° C. overnight. The binding reaction is stopped by incubation of the nanodecoy-bead mixture with an equal volume of 200 nM glycine for 30 mins at room temperature followed by two washes with MACS flow buffer. Nanodecoy bound beads are then incubated with ACE2 (PA5-85139; Invitrogen) and SFPTC (AB3786; Sigma-Aldrich) antibodies for 1 hour at 4° C. followed by two washes with MACS flow buffer. Fluorescent secondary antibodies (A32731; Thermo Fisher) were then incubated for 1 hour in the dark at 4° C. followed by one wash with MACS flow buffer. Plain beads and unstained nanodecoy bond beads were used as controls. Flow cytometry was performed on the CytoFlex (Beckman Coulter) or LSR-II (BD) and analyzed using FCS Express V6 (De Novo Software) or FACSDiva Software (BD).

SARS-CoV-2 stock. The SARS-CoV-2 USA-WA1/2020 stock was expanded from the BEI Resource (NR-52281; Lot #70033175; courtesy Natalie Thornburg, Centers for Disease Control and Prevention) in Vero E6 cells and harvested the virus challenge stock on day 5 following infection at 90% cytopathic effect (CPE). Full genome sequencing revealed 100% identity with the parent virus sequence (GenBank MN985325.1; courtesy David O'Connor, Shelby O'Connor, University of Wisconsin).

Histopathology and immunohistochemistry of macaques' lung tissues. Tissues were fixed in freshly prepared 4% PFA for 24 hours, transferred to 70% ethanol, and paraffin embedded within 7 days and blocked sectioned at 5 μm. Slides were then baked for 60 mins at 65° C., deparaffinized in xylene, and rehydrated through a series of graded ethanol to distilled water. Subsequently, the slides were stained with hematoxylin (HSS16, Sigma-Aldrich) and eosin Y (318906, Sigma-Aldrich). An optical microscopy was performed to analyze these slides. For SARS nucleocapsid protein (SARS-N) of immunohistochemistry (IHC) staining, retrieval was performed in citrate buffer first (AP9003125, Thermo) and followed by treated with 3% H₂O₂ in methanol. Slides were permeabilized and blocked with Dako protein blocking solution (X0909, DAKO) containing 0.1% saponin (47036, Sigma-Aldrich). Primary rabbit anti-SARS-N antibody (NB100-56576, Novus, 1:200) was incubated overnight at 4° C. and then goat anti-rabbit HRP secondary antibody (ab6721, Abcam, 1:1000) was incubated for 30 minutes and then counterstained with hematoxylin followed by bluing using 0.25% ammonia water.

Subgenomic mRNA assay. SARS-CoV-2 E gene subgenomic mRNA (sgRNA) was assessed by RT-PCR. To generate a standard curve, the SARS-CoV-2 E gene sgRNA was cloned into a pcDNA3.1 expression plasmid; this insert was transcribed using an AmpliCap-Max T7 High Yield Message Maker Kit (Cellscript) to obtain RNA for standards. Prior to RT-PCR, samples collected from challenged animals or standards were reverse-transcribed using Superscript III VILO (Invitrogen) according to the manufacturer's instructions. A Taqman custom gene expression assay (ThermoFisher Scientific) was designed using the sequences targeting the E gene sgRNA. Reactions were carried out on a QuantStudio 6 and 7 Flex Real-Time PCR System (Applied Biosystems) according to the manufacturer's specifications. Standard curves were used to calculate sgRNA in copies per mL or per swab; the quantitative assay sensitivity was 50 copies per mL or per swab.

RNAscope in situ hybridization. RNAscope in situ hybridization was performed using SARS-CoV-2 anti-sense specific probe v-nCoV2019-S (ACD Cat. No. 848561) targeting the positive-sense viral RNA, SARS-CoV-2 sense specific probe vnCoV2019-orf1ab-sense (ACD Cat. No. 859151) targeting the negative-sense genomic viral RNA, and ZIKA probe V-ZIKVsph2015 (ACD Cat. No. 467871) as a negative control. Briefly, slides were deparaffinized in xylene first and then rehydrated through a series of graded ethanol to distilled water followed by incubating with RNAscope® H₂O₂(ACD Cat. No. 322335) for 10 mins at room temperature, retrieval was performed for 15 mins in ACD P2 retrieval buffer (ACD Cat. No. 322000) at 95-98° C., followed by treatment with protease plus (ACD Cat. No. 322331) for 30 min at 40° C. Probe hybridization and detection were developed using the RNAscope® 2.5 HD Detection Reagents-RED (ACD Cat. No. 322360) according to the manufacturer's instructions.

Immunofluorescence staining of macaques' lung tissues. In brief, the pretreatment of slides is the same with IHC assay including dewaxing, rehydration, retrieval and 3% H₂O₂ treatment. After that, slides were first blocked with 5% BSA for 30 mins followed by rinse in PBS buffer. Primary rabbit anti-SARS-N antibody (1:200) incubated overnight at 4° C. and then goat anti-rabbit Alexa Fluor® 594 (Abcam, ab150080, 1:500), AF-488-CD206 (Santa Cruz Biotechnologies, sc-376108, 1:150), eFluor 660-CD68 (eBioscience, 50-0681-82, 1:200) and Alexa Fluor® 568-Iba-1 (Abcam, ab221003, 1:200) were incubated at RT for 1 hr. For co-localization assay, the FITC-pan-CK (abcam, ab78478, 1:200) was incubated at RT for 1 hr after the incubation of SARS-N. All slides were imaged on the Olympus FLUOVIEW confocal microscope.

SARS-CoV-2 Vaccine Based on Exosome VLPs

Severe acute respiratory syndrome (SARS-CoV-2) infection has progressed into the worldwide pandemic disease. Effective vaccination is the only way to control and eliminate this pandemic. The first two vaccines approved by FDA are mRNA vaccines. They need to be deeply frozen (−70 or −20° C.) during transportation and storage, and need to be administered via injection, creating excessive burdens to the already struggling healthcare system in this pandemic. Embodiments of the present disclosure provide a lyophilizable, room temperature-stable, and inhalable vaccine against SARS-CoV-2. Recombinant RBD was conjugated onto lung-derived exosomes (Exo) to make a virus-like particle (VLP) vaccine. As described further herein, results demonstrated the advantages of using Exo over liposomes, with augmented retention in both mucus-lined respiratory airway and lung parenchyma. Results further demonstrated that after lyophilization RBD-Exo VLP vaccine was stable after 21-day of storage at room temperate. Inhalation of RBD-Exo VLP triggered both RBD-specific IgG and IgA responses to clear SARS-CoV-2 mimic virus in a mouse model. In a hamster model of live SARS-CoV-2 infection, two doses of RBD-Exo ameliorate SARS-CoV-2 infection, attenuate severe pneumonia, and reduce inflammatory infiltrates.

Cell Culture. Lung spheroid cells (LSCs) were generated from healthy human whole lung samples from the Cystic Fibrosis and Pulmonary Diseases Research and Treatment Center at the University of North Carolina at Chapel Hill and expanded as previously described. LSCs were plated on a fibronectin-coated (Corning Incorporated, Corning, N.Y., USA) flask and maintained in Iscove's Modified Dulbecco's Media (IMDM; ThermoFisher Scientific, Waltham, Mass., USA) containing 20% fetal bovine serum (FBS; Corning Incorporated, Corning, N.Y., USA). Media changes were performed every other day. LSCs were allowed to reach 70-80% confluence before generating serum-free secretome (LSC-Secretome) as previously described. LSC-Secretome was collected and filtered through a 0.22 μm filter to remove cellular debris. Murine macrophage RAW 264.7 cells (ATCC, Manassas, Va., USA) were purchased and maintained in Dulbecco's Modified Eagle Medium (DMEM; ThermoFisher Scientific, Waltham, Mass., USA) media containing 10% FBS (Corning Incorporated, Corning, N.Y., USA). Media changes were performed every other day. RAW 264.7 cells were allowed to reach 70-80% confluence before co-culturing with RBD and RBD-Exo. Splenocytes were isolated from vaccinated mice as previously described. All procedures performed in the present disclosure involving human samples were in accordance with the ethical standard of the institutional research committee and with the guidelines set by the Declaration of Helsinki.

Exosome Isolation and characterization. Exosomes were collected and isolated from human LSC-Secretome via ultrafiltration. Filtered secretome was pipetted into a 100 kDa Amicon centrifugal filter unit (MilliporeSigma, Burlington, Mass., USA) and centrifuged at 400 RCF at 10° C. Once all media was filtered, the remaining exosomes were detached from the filter and resuspended using Dulbecco's phosphate-buffered saline (DPBS; ThermoFisher Scientific, Waltham, Mass., USA) with 25 mM Trehalose (MilliporeSigma, Burlington, Mass., USA) for further analysis. LSC-Exo, RFP-Exo, RFP-Lipo, and RBD-Exo were quantified by nanoparticle tracking analysis (NTA; NanoSight NS3000, Malvern Panalytical, Malvern, UK). All samples were fixed onto copper grids and stained with vanadium negative staining for TEM (JEOL JEM-2000FX, Peabody, Mass., USA), to analyze exosomal internal composition and morphology before and after RBD binding. To determine the presence of RBD through TEM, RBD-Exo were incubated with anti-RBD primary antibody (NC1792214; Fisher Scientific, Pittsburgh, Pa., USA) overnight at 4° C. Unbound antibodies were removed via ultracentrifugation at 100,000 g for 30 minutes. Gold nanoparticles (15 nm) labeled with goat anti-rabbit IgG secondary antibody were added and incubated at room temperature for 2 hours.

RBD conjugation on LSC-Exosomes. Recombinant SARS-CoV-2 RBD protein (Sino Biological, Beijing, China) was purchased and reconstituted in DPBS. RBD was conjugated to LSC-Exo using a DSPE-PEG-NHS linker by co-incubation for 24 hours at 4° C. To quantify the RBD moiety on LSC-Exo, RBD-Exo were resuspended in 100 μL deionized water, and ultrasonicated to lyse the exosomes. The amount of released RBD was quantified via ELISA.

SDS-PAGE and western blot. Samples were further characterized through immunoblotting for the presence of exosome markers CD63 (PA5-100713; ThermoFisher Scientific, Waltham, Mass., USA) and RBD (NC1792214; Fisher Scientific, Pittsburgh, Pa., USA). Samples were lysed, denatured, and reduced by Laemmli sample buffer (Bio-Rad, Hercules, Calif., USA) and f-mercaptoethanol (Bio-Rad, Hercules, Calif., USA) at 90° C. for 5 minutes. Protein samples and molecular ladder (Precision Plus Protein Unstained Standards; Bio-Rad, Hercules, Calif., USA) were loaded into a 10% acrylamide precast Tris-Glycine gel (Bio-Rad, Hercules, Calif., USA) for sodium dodecyl sulfate polyacrylamide gel electrophoresis (SDS-PAGE) separation. Gels were run at a stacking voltage of 100V until samples ran out of the wells, followed by a constant voltage of 200V. Gels were visualized and imaged by UV light in a Bio-Rad Imager (Bio-Rad, Hercules, Calif., USA). Gels were transferred onto PVDF membranes (Bio-Rad, Hercules, Calif., USA) using the Bio-Rad wet electroblotting transfer system (Bio-Rad, Hercules, Calif., USA). Following transfer, membranes were washed three times in 1×phosphate-buffered saline with 0.1% Tween detergent (PBS-T; MilliporeSigma, Burlington, Mass., USA) for 5 minutes each and blocked using 5% milk in PBS-T for one hour at room temperature. Membranes were blotted against primary antibodies in 5% milk in PBS-T. Primary antibodies were incubated at 4° C. for one week. After incubation, membranes were incubated with the corresponding HRP-conjugated secondary antibodies for 1.5 hours at room temperature. Membranes were then visualized and imaged by UV light in a Bio-Rad Imager (Bio-Rad, Hercules, Calif., USA).

Stability studies on RBD-Exo VLPs. RBD-Exo lyophilizates were stored at −80° C., 4° C. and room temperature for 21 days. Then, RBD-Exo lyophilizate were dispersed in PBS and the size and concentration were detected using NTA. In addition, the concentration of RBD on Exo were quantified using ELISA kit.

RBD-Exo internalization by APCs. RBD was labeled using NHS-Rhodamine (ThermoFisher Scientific, Waltham, Mass., USA) according to manufacturer's protocol. RBD-RhB and RBD-RhB-Exo were co-cultured with RAW264.7 cells for 1 hour with the same concentrations of RBD (1 μg). The free RBD-RhB and RBD-RhB-Exo were removed and cells were washed three times with DPBS. Cells were imaged with an Olympus FLUOVIEW CLSM (Olympus; FV3000, Shinjuku, Tokyo, Japan).

Synthesis of SARS-CoV-2 mimics. Spike protein (Sino Biological, Beijing, China) was conjugated to lentivirus (Cellomics Technology LLC, Halethorpe, Md., USA) to create a SARS-CoV-2 mimic, according to previous reports. His-tagged Spike protein was bind to Ni nitrilotriacetate (Ni-NTA) through the chemical interaction. NTA with mercapto group (N-[Nα,Nα-Bis(carboxymethyl)-L-lysine]-12-mercaptododecanamide) were first reacted with 4-(N-Maleimidomethyl)cyclohexane-1-carboxylic acid 3-sulfo-N-hydroxysuccinimide ester sodium salt (Sulfo-SMCC) to give NTA-SMCC and then were added to the lentivirus. The NTA groups were conjugated to the lentivirus through the —NH₂ group on lentivirus and N-hydroxysuccinimide ester on NTA-SMCC. The free NTA-SMCC was removed by centrifugation using a 100 kDa Amicon centrifugal filter unit (MilliporeSigma, Burlington, Mass., USA) to give SARS-CoV-2 mimicking virus (S-protein-lentivirus). Briefly, SARS-CoV-2 mimics were incubated with anti-Spike protein antibodies overnight at 4° C. The gifting efficiency of spike protein to lentivirus were measured using ELISA. In brief, SARS-CoV-2 mimics (10⁶ transducing units (TU)/mL) were lysed, and the lysates were homogenized and measured using ELISA kit (Sino Biological SARS-CoV-2 SPIKE ELISA KIT, Sino Biological, Beijing, China) according to manufacturer's protocol.

Flow Cytometry. Antigen internalization by APCs was further characterized through flow cytometry. Cells were washed with MACS flow buffer (Miltenyi Biotec, Bergisch Gladbach, Germany) and permeabilized with BD Cytofix/Cytoperm (BD Biosciences, San Jose, Calif., USA) prior to incubation with antibodies against CD86-APC (565479; BD Biosciences, San Jose, Calif., USA), CD40-PE (553791; BD Biosciences, San Jose, Calif., USA), and CD80-APC (A14724; Invitrogen, Waltham, Mass., USA). Samples were gated with CD11b (53-0112-82; eBioscience, San Diego, Calif., USA). Flow cytometry was performed on the CytoFLEX flow cytometer (Beckman Coulter, Brea, Calif., USA) and analyzed using FCS Express V6 (De Novo Software; denovosoftware.com).

Mouse studies using SARS-CoV-2 mimics. All studies complied with the requirements of the Institutional Animal Care and Use Committee. Seven-week-old male CD1 mice (Crl:CD1(ICR)) were obtained from Charles River Laboratory (Wilmington, Mass., USA). RFP-Exo and RFP-Lipo were administered via nebulization (Pari Trek S Portable 459 Compressor Nebulizer Aerosol System; 047F45-LCS, PARI, Starnberg, Germany). PBS, Exo, RBD, and RBD-Exo treatments were given in two doses once a week for two weeks via nebulization or IV injection. Mice were challenged with AF647 labeled SARS-CoV-2 mimics (10⁶ particles per kg of body weight) by nebulization one week after the second treatment dose. Lung organs were collected and imaged at day 2 and day 6 post vaccination with an Xenogen Live Imager (PerkinElmer, Waltham, Mass., USA). Blood and major organs were collected for further analysis.

Histology studies in mice. Immunostaining was performed on tissue slides fixed in 4% paraformaldehyde (PFA; Electron Microscopy Sciences, Hatfield, Pa., USA) in DPBS, followed by permeabilization and blocking with Dako Protein blocking solution (Agilent Technologies, Santa Clara, Calif., USA) with 0.1% saponin (Sigma-Aldrich, St. Louis, Mo., USA). Cells were stained with antibodies against Phalloidin (ab176753; Abcam, Cambridge, United Kingdom) and CD11b (ab216524; Abcam, Cambridge, United Kingdom). Slides were mounted with ProLong Gold Antifade Mountant with 4′,6-diamidino-2-phenylindole (Invitrogen, Waltham, Mass., USA) and imaged on the Olympus FLUOVIEW CLSM (Olympus; FV3000, Shinjuku, Tokyo, Japan) with an Olympus UPlanSAPO 10× objective (Olympus; 1-U2B824, Shinjuku, Tokyo, Japan) and Olympus UPlanSAPO 60× objective (Olympus; 1-U2B832, Shinjuku, Tokyo, Japan).

IgG antibody titer. Micro titer plates (Nunc Cell Culture, ThermoFisher Scientific, Waltham, Mass., USA) were coated with 100 μL of 10 μg/mL RBD in coating buffer (R&D Systems, Minneapolis, Minn., USA) and incubated overnight at 4° C. To reduce nonspecific binding, wells were blocked with 200 μL of 1% (w/v) bovine serum albumin (BSA; Sigma-Aldrich St. Louis, Mo., USA) in PBS-T for 1 hour at 37° C. After extensive washing with PBS-T, serial dilutions (1:100, 1:1000, 1:5000, 1:10000, 1:50000, 1:100000, 1:200000) of sera samples were added and control sera samples were diluted to 1:100. After incubation for 1.5 hours at 37° C., samples were washed three times with PBS-T and incubated with HRP-labeled anti-Mouse IgG secondary antibody at a 1/2000 dilution (100 μL per well) or HRP-labeled anti-Hamster IgG secondary antibody at a 1/20000 dilution (100 μL per well) for 1 hour at 37° C. Samples were washed four times with PBS-T and 3,3′,5,5′-tetramethylbenzidine soluble substrate (TMB; ThermoFisher Scientific, Waltham, Mass., USA) soluble substrate was added to each well (100 μL per well). After a 30-minute incubation at room temperature, the color development was stopped by adding 50 μL of stop solution (2 M H₂SO₄, Sigma-Aldrich St. Louis, Mo., USA) and optical absorption was measured at 450 nm on a plate reader. The end-point titer of IgG was determined by the reciprocal of maximal serum dilution that exceeded twice the SD above the mean control group optical density. The individual antibody titers were expressed as [log 10[X̅±SD]], calculated as the reciprocal of maximal serum dilution.

IgA antibody titer. RBD-specific IgA from NPLF and BALF was measured using an ELISA. To collect NPLF, the trachea was cut in the middle and the nasopharynx was rinsed upwards from the incision with 200 μL DPBS. The fluid was collected and the rinse was repeated three times for a total of 600 μL wash fluid. To collect BALF, the trachea was exposed by thoracotomy and a transverse incision was made at the top of the bronchial bifurcation. A needle was inserted into the trachea to wash the lungs with 200 μL of DPBS. The wash fluid was collected and the rinse was repeated three times for a total of 600 μL wash fluid. Micro titer plates (Nunc Cell Culture, ThermoFisher Scientific, Waltham, Mass., USA) were coated with 100 μL of 5 μg/mL RBD in coating buffer (R&D Systems, Minneapolis, Minn., USA) and incubated overnight at 4° C. To reduce nonspecific binding, wells were blocked with 200 μL of 1% (w/v) BSA (Sigma-Aldrich St. Louis, Mo., USA) in PBS-T for 1 hour at 37° C. After extensive washing with PBS-T, serial dilutions (1:50, 1:100, 1:1000, 1:5000, 1:10000, 1:50000) of NPLF and BALF were added and control samples were diluted to 1:50. After incubation for 1.5 hours at 37° C., samples were washed three times with PBS-T, and incubated with HRP-labeled anti-Mouse IgA secondary antibody at a 1/2000 dilution (100 μL per well) for 1 hour at 37° C. Samples were washed four times with PBS-T and TMB soluble substrate (ThermoFisher Scientific, Waltham, Mass., USA) soluble substrate was added to each well (100 μL per well). After a 30-minute incubation at room temperature, the color development was stopped by adding 50 μL of stop solution (2 M H₂SO₄, Sigma-Aldrich St. Louis, Mo., USA) and optical absorption was measured at 450 nm on a plate reader. The end-point titer of IgA was determined by the reciprocal of maximal serum dilution that exceeded twice the SD above the mean control group OD. The individual antibody titers were expressed as [log 10[X̅±SD]] and calculated as the reciprocal of maximal serum dilution.

Cytokine measurement in splenocytes. Splenocytes from each vaccinated mouse were challenged with 1 μg/mL RBD and plated into ELISPOT wells (10⁶ per well) (R&D Systems, Minneapolis, Minn., USA) that were coated with anti-mouse IFN-γ capture antibody. Antigen-specific cells secreting IFN-γ were detected using an ELISPOT Assay according to manufacturers' protocol. SFUs were analyzed using an anatomical microscope (Nikon, Minato City, Tokyo, Japan) and the spots were counted using ImageJ software (NIH; imagej.nih.gov/ij/). Splenocytes from each vaccinated mouse were cultured in 6-well plates (5×10⁶ cells per well) and re-stimulated with 5 μg/mL RBD. After a 48-hour incubation, antigen-specific cytokine levels of IL-6 and TNF-α from culture medium were detected by ELISA using Mouse IL-6 ELISA Kit (RAB0308, Sigma-Aldrich St. Louis, Mo., USA) and Mouse Tumor Necrosis Factor α ELISA Kit (RAB0477, Sigma-Aldrich, St. Louis, Mo., USA) per manufacturer's protocols. Splenocytes were collected after removing culture medium and were fixed in 4% PFA (Electron Microscopy Sciences, Hatfield, Pa., USA) and stained with anti-mouse CD11b-AF488 (53-0112-82; eBioscience, San Diego, Calif., USA).

Live SARS-CoV-2 stock. The SARS-CoV-2 USA-WA1/2020 stock was expanded from the BEI Resource (NR-52281; Lot #70033175; courtesy Natalie Thornburg, Centers for Disease Control and Prevention) in Vero E6 cells, and harvested the virus challenge stock on day 5 following infection at 90% cytopathic effect (CPE). Full genome sequencing revealed 100% identity with the parent virus sequence (GenBank MN985325.1; courtesy David O'Connor, Shelby O'Connor, University of Wisconsin).

Hamster studies with live SARS-CoV-2. Fifteen male and female Syrian golden hamsters (Envigo), 6-8 weeks old, were randomly allocated to three treatment groups. All animals were housed at Bioqual Inc. Hamsters were administered with two doses of PBS (placebo), RBD or RBD-Exo 1 week apart by inhalation using a nebulization and fitted mask (n=5 per group, 3F/2M). 1 week after the second dose of vaccine, the hamsters were challenged with 100 μl of SARS-CoV-2 (5.5×10⁵ PFU) using the intranasal and intratracheal routes (50 μl in each nare). Bronchoalveolar lavage (BAL), oral swabs (OS) and blood were monitored or collected at the indicated time. Hamsters were necropsied on day 7 post-challenge. All immunologic and virologic assays were performed blinded. All animal studies were conducted in compliance with all relevant local, state, and federal regulations and were approved by the Bioqual Institutional Animal Care and Use Committee (IACUC).

Histopathology and immunohistochemistry in infected hamsters. Tissues were fixed in freshly prepared 4% paraformaldehyde for 24 hours, transferred to 70% ethanol, and paraffin embedded within 7 days and blocked sectioned at 5 μm. Slides were then baked for 60 mins at 65° C. and deparaffinized in xylene and rehydrated through a series of graded ethanol to distilled water. Subsequently, the slides were stained with hematoxylin (HSS16, Sigma-Aldrich) and eosin Y (318906, Sigma-Aldrich). Trichrome (HT10516, Sigma-Aldrich) staining was also performed according to manufacturer's instructions. An optical microscopy was performed to analyze these slides. For SARS-N, CD3, MPO and MX1 of IHC staining, retrieval was performed using in citrate buffer first (AP9003125, Thermo) and followed by treatment with 3% H₂O₂ in methanol for 10 mins after dewaxing and rehydration. Slides were permeabilized and blocked with Dako Protein blocking solution (X0909, DAKO) containing 0.1% saponin (47036, Sigma-Aldrich). Primary rabbit anti-SARS-N antibody (Novus, NB100-56576, 1:200), rabbit anti-CD3 (Abcam, ab16669, 1:200), rabbit anti-MPO (Thermo, PA5-16672, 1:200) and anti-MX1 (Millipore Sigma, MABF938, 1:200) were incubated overnight at 4° C., respectively and followed by goat anti-rabbit HRP secondary antibody (Abcam, ab6721, 1:1000) or goat anti-mouse HRP secondary antibody (Abcam, ab6789, 1:1000) were incubated for 1 hr at RT and then counterstained with hematoxylin followed by bluing using 0.25% ammonia water.

Viral load assay in hamsters. SARS-CoV-2 RNA copies per milliliter (copies/mL) was determined by a two-step real-time quantitative PCR assay developed in the Clinical Laboratory Improvement Amendments-certified Immunology and Virology Quality Assessment Center at the Duke Human Vaccine Institute. DSP Virus/Pathogen Midi Kits (Qiagen, Hilden, Germany) were used to extract viral RNA on a QIAsymphony SP automated sample preparation platform. A reverse primer specific to the SARS-CoV-2 envelope gene was annealed to the extracted RNA and reverse transcribed into cDNA using SuperScript III Reverse Transcriptase and RNaseOut (Thermo Fisher Scientific, Waltham, Mass.). cDNA was treated with RNase H and then added to a custom 4× TaqMan Gene Expression Master Mix (Applied Biosystems, Foster City, Calif.) containing envelope gene-specific primers and a fluorescently labeled hydrolysis probe; quantitative PCR was carried out on a QuantStudio 3 Real-Time PCR system (Thermo Fisher Scientific, Waltham, Mass.). SARS-CoV-2 RNA copies per reaction were interpolated using quantification cycle data and a serial dilution of a highly characterized custom DNA plasmid containing the SARS-CoV-2 envelope gene sequence. The limit of quantification was 62 RNA copies/mL of sample as determined by an extensive validation process consistent for use in a clinical setting.

RNAscope in situ hybridization in hamsters. RNAscope in situ hybridization was performed using SARS-CoV-2 anti-sense specific probe v-nCoV2019-S(ACD Cat. No. 848561) targeting the positive-sense of Spike sequence, SARS-CoV-2 v-nCoV2019-S-sense (ACD Cat. No. 845701) targeting the negative-antisense of Spike sequence. Briefly, slides were deparaffinized in xylene first and then rehydrated through a series of graded ethanol to distilled water followed by incubating with RNAscope® H₂O₂(ACD Cat. No. 322335) for 10 mins at room temperature, retrieval was performed for 15 mins in ACD P2 retrieval buffer (ACD Cat. No. 322000) at 95-98° C., followed by treatment with protease plus (ACD Cat. No. 322331) for 30 min at 40° C. Probe hybridization and detection were developed using the RNAscope® 2.5 HD Detection Reagents-RED (ACD Cat. No. 322360) according to the manufacturer's instructions.

Immunofluorescence staining of hamster lung sections. In brief, the pretreatment of slides is the same with IHC assay including dewaxing, rehydration, retrieval and 3% H₂O₂ treatment. After that, slides were first blocked with 5% BSA for 30 mins followed by rinses with DPBS for 3 times. Primary rabbit anti-SARS-N antibody (1:200) incubated overnight at 4° C. and followed by goat anti-rabbit Alexa Fluor®647 (Abcam, ab150080, 1:500), AF-488-CD206 (Santa Cruz Biotechnologies, sc-376108, 1:150) and Alexa Fluor® 568-Iba-1 (Abcam, ab221003, 1:200) were incubated at RT for 1 hr, or followed by goat anti-rabbit Alexa Fluor® 647 (Abcam, ab150079, 1:500) and FITC-pan-CK (abcam, ab78478, 1:200) was incubated at RT for 1 hr. Finally, all the slides were mounted with ProLong Gold Antifade Mountant with 4′,6-diamidino-2-phenylindole (Invitrogen, Waltham, Mass., USA) and imaged on the Olympus FLUOVIEW CLSM (Olympus; FV3000, Shinjuku, Tokyo, Japan).

Statistical analysis. All experiments were performed at least three times independently. Results are shown as means±standard deviation. Comparisons between any two groups were performed using the two-tailed, unpaired Student's t-test. Comparisons among more than two groups were performed using one-way ANOVA, followed by the post hoc Bonferroni test. Single, double, triple and four asterisks represent p<0.05, 0.01, 0.001, and 0.0001, respectively; p<0.05 was considered statistically significant.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

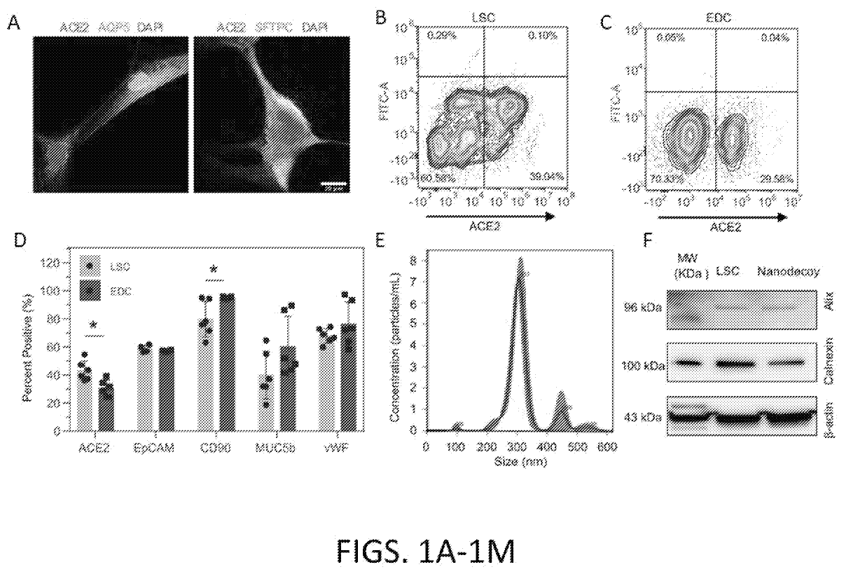

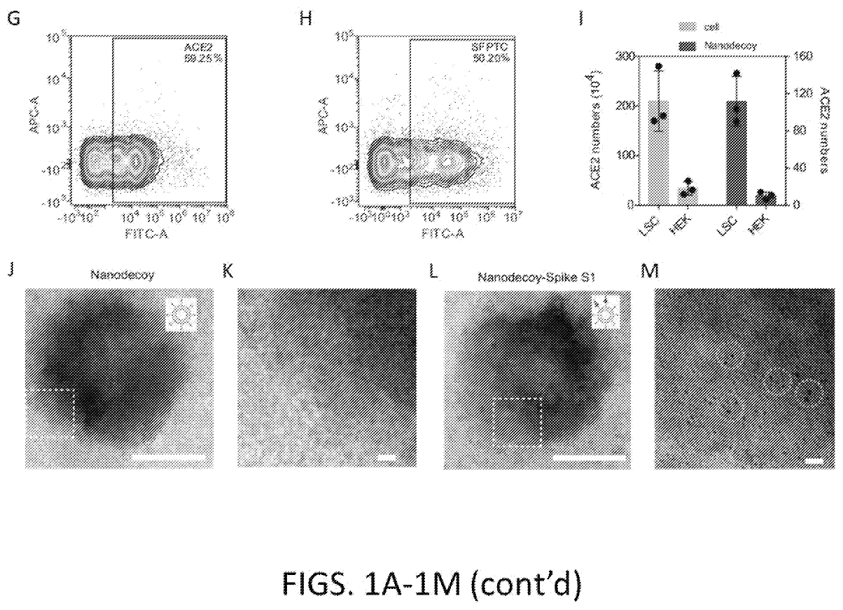

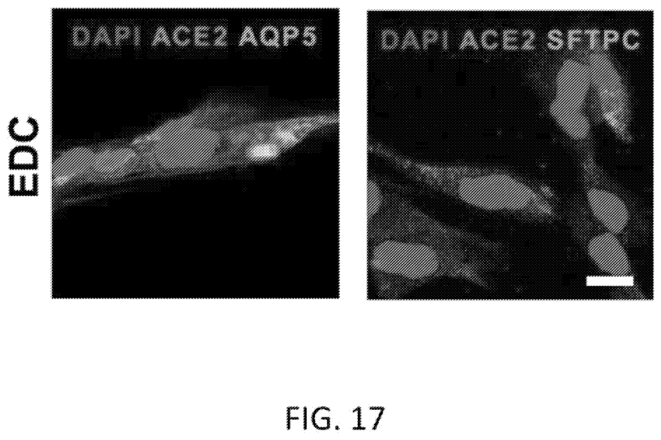

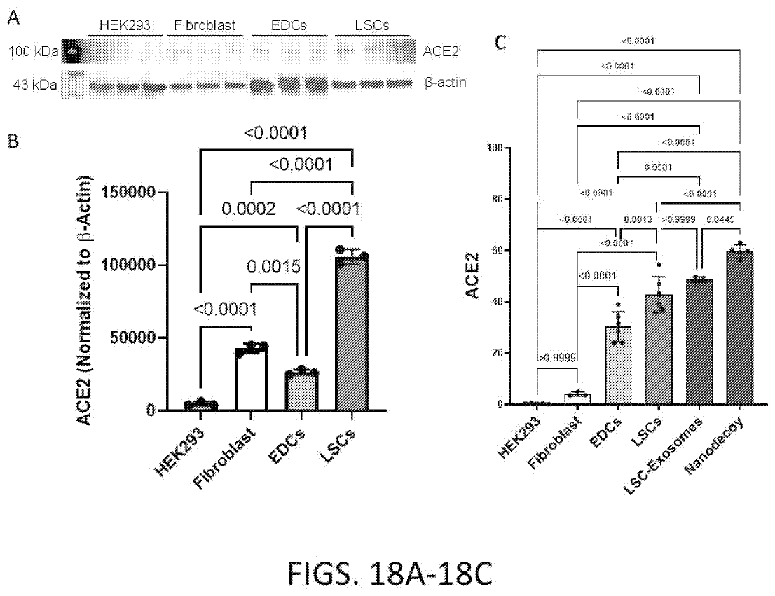

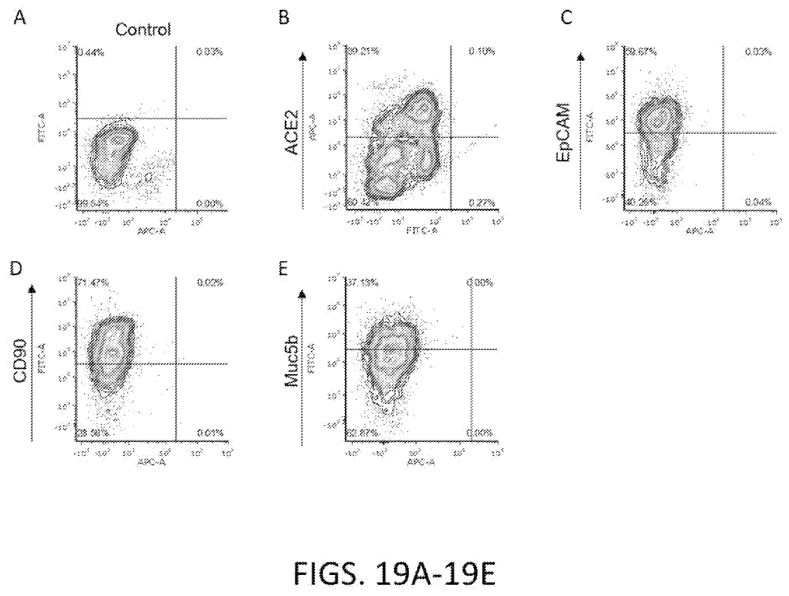

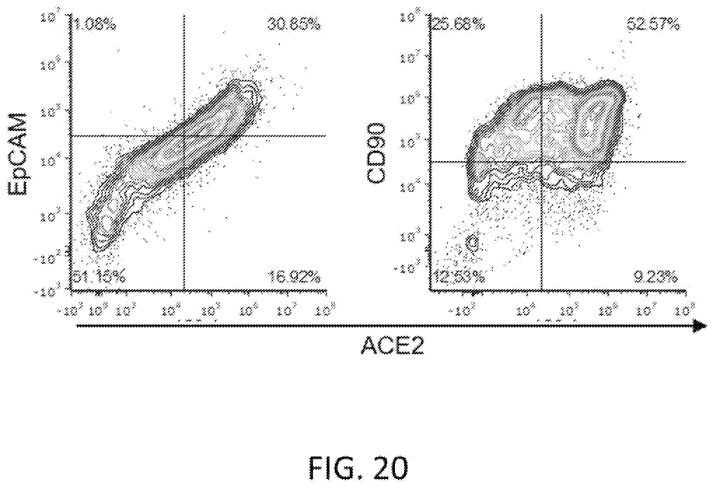

Fabrication of LSC-nanodecoys. The overall rationale of the nanodecoy design is shown in FIG. 16. First, LSCs and their parent cells, lung explant-derived cells (EDCs), were screened for ACE2 expression to determine the optimal cell types for nanodecoy fabrication. LSCs and EDCs were analyzed by immunostaining (FIG. 1A; FIG. 17), immunoblotting, and flow cytometry (FIGS. 1B-1C; FIG. 18) for ACE2 expression. In addition, the ACE2 expression levels of HEK293 and human lung fibroblasts were studied using immunoblotting and flow cytometry as controls (FIG. 18). LSCs were found to have higher ACE2 expression levels than the other cell types, including their parent cell, EDCs. In comparison, HEK293 and fibroblasts had visibly lower ACE2 expression. Consistent with previous studies, confocal imaging showed that ACE2 was present on the membrane of AQP5⁺ type I pneumocytes and SFTPC⁺ type II pneumocytes (FIG. 1A), two subpopulations within LSCs. Analysis found ACE2 was co-expressed with other LSC makers such as EpCAM, CD90, and MUC5b (FIG. 1D and FIGS. 19-20). Previous studies have indicated that 83% of ACE2-expressing cells in lung tissue are type II pneumocytes, suggesting that the lungs are the most vulnerable target organs to the SARS-CoV-2 virus. Thus, these results demonstrated that, as primary resident lung cells, LSCs might serve as an ideal cell type to generate nanodecoys with high levels of ACE2 expression. In contrast, HEK293 cells were used as a control for preparing nanodecoys with a low level of ACE2 expression.

LSC and HEK293 membrane nanovesicles (nanodecoys) were generated by serial extrusion of LSCs or HEK293 cells through polycarbonate membranes with pore sizes of 5 μm, 1 μm, and finally, 0.4 μm with a commercial extruder. The obtained LSC-nanodecoys were characterized by Nanoparticle Tracking Analysis, showing a homogeneous nanoparticle population with an average size distribution of 320 nm and an average quantity of 5.51×10¹⁰ particles/mL produced from 5×10⁶ cells (FIG. 1E). In other words, on average, one LSC generated 11,020 nanodecoys. Because whole cells were used to prepare the nanodecoys, it was hypothesized that the nanodecoys were not exclusively generated from the plasma membranes but also from intracellular membranes. To confirm this hypothesis, the intracellular component of the nanodecoys was investigated by testing for Alix (a phylogenetically conserved cytosolic scaffold protein) and Calnexin (a marker of endoplasmic reticulum). Results showed that these two intracellular markers were detected, which supported the analysis (FIG. 1F). Flow cytometry analysis confirmed the preservation of ACE2 on the surface of the nanodecoys (FIG. 1G) as well as type II pneumocyte marker SFTPC (FIG. 1H). Moreover, the quantity of ACE2 on both LSCs and HEK293 cells was investigated, along with their nanodecoys by ELISA analysis. The frequency of ACE2 was determined to be 2.1×10⁶ receptors per LSC and 112 receptors per LSC-nanodecoy. In stark contrast, 3.4×10⁵ and 10 ACE2 receptors were found to be present on each HEK293 cell and HEK-nanodecoy, respectively (FIG. 1I). Furthermore, transmission electron microscope (TEM) images revealed the spherical morphology of nanodecoys (FIGS. 1J-1K).

### Example 2

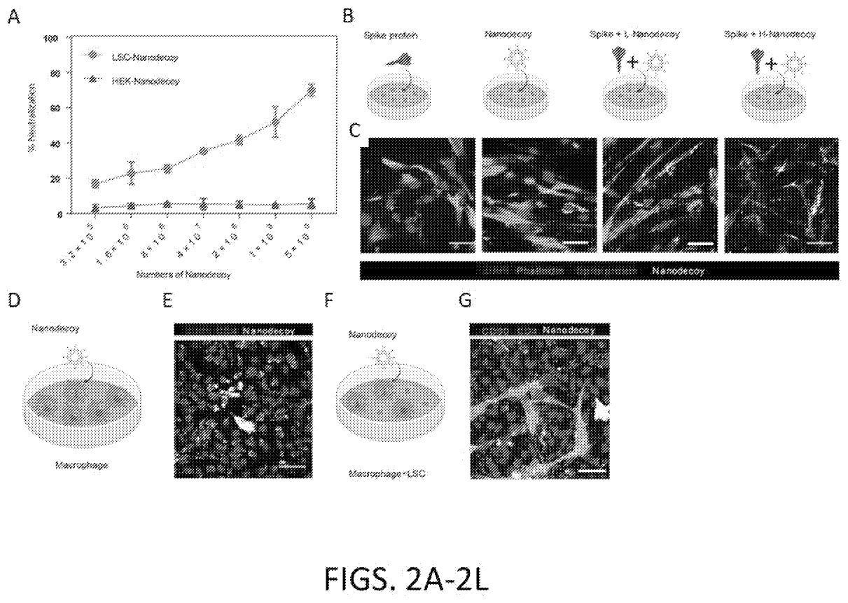

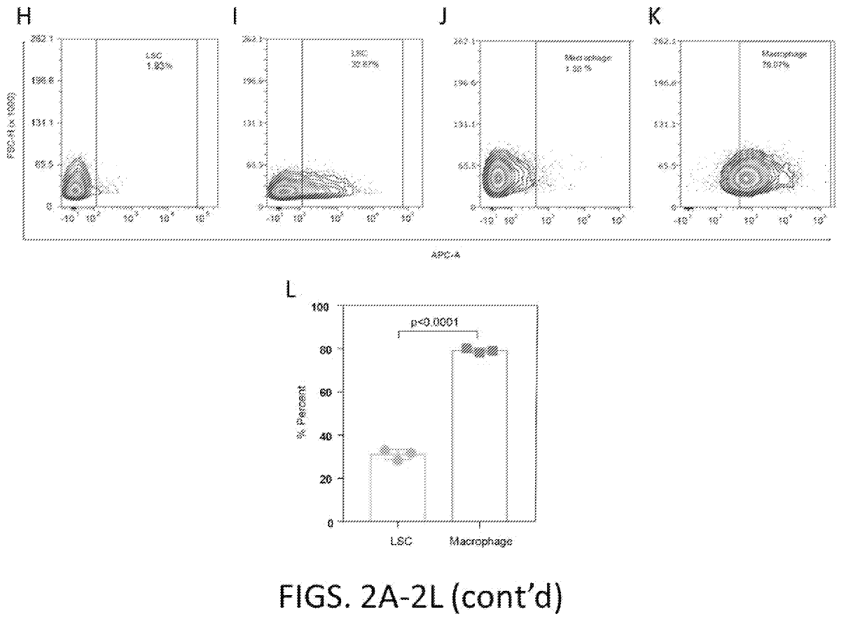

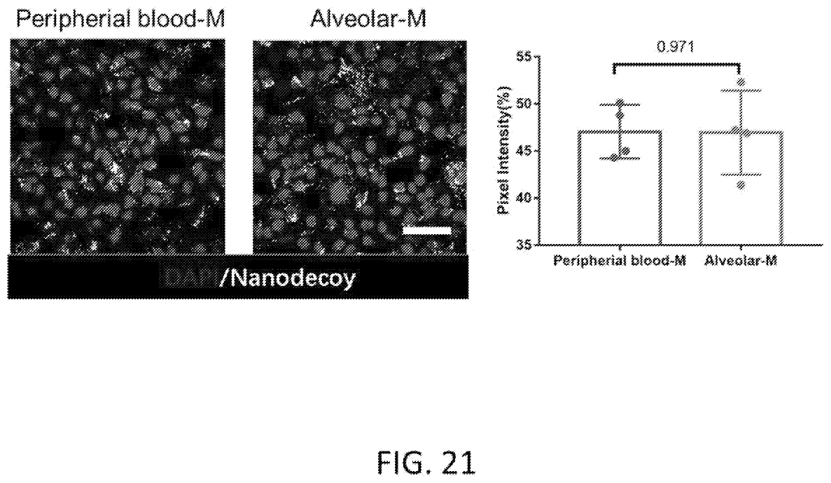

Nanodecoys can bind and neutralize S-protein in-vitro. Having demonstrated the presence of ACE2 on the nanodecoys, their ability to bind the SARS-CoV-2 S-protein was then tested. Spike S1 of the spike protein contains a receptor-binding domain (RBD) that specifically recognizes ACE2. Therefore, it was first confirmed that spike S1 could bind to the nanodecoys by TEM with immunogold labeling (FIGS. 1L-1M). In a dose-responsive manner, 50% of spike S1 (6.5×10¹⁰) was captured and bound by 109 LSC-nanodecoys, whereas nanodecoys derived from HEK293 cells failed to bind to spike S1 (FIG. 2A). The binding potency of LSC- and HEK293-nanodecoys was then examined using lung cell-based assays (FIG. 2B). Spike S1 was found to bind to lung cells after 4 hours of incubation (FIG. 2C). DiD-labeled LSC-nanodecoys co-localized with spike S1, while HEK293-nanodecoys did not, suggesting that the LSC-nanodecoys could recognize and competitively bind to spike S1. Additionally, macrophages had a greater internalization efficiency of the nanodecoys than the lung cells did (FIGS. 2D-2G), indicating the potential clearance of nanodecoys and their neutralized SARS-CoV-2 by macrophages and/or other immune cells, which was confirmed by flow cytometry analysis (FIGS. 2H-2L). Furthermore, both peripheral blood and alveolar macrophages had the same internalization rate of LSC-nanodecoys (FIG. 21).

### Example 3

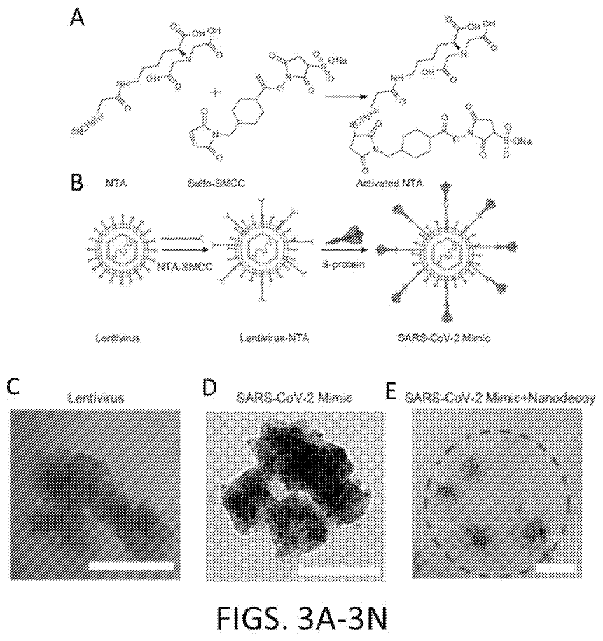

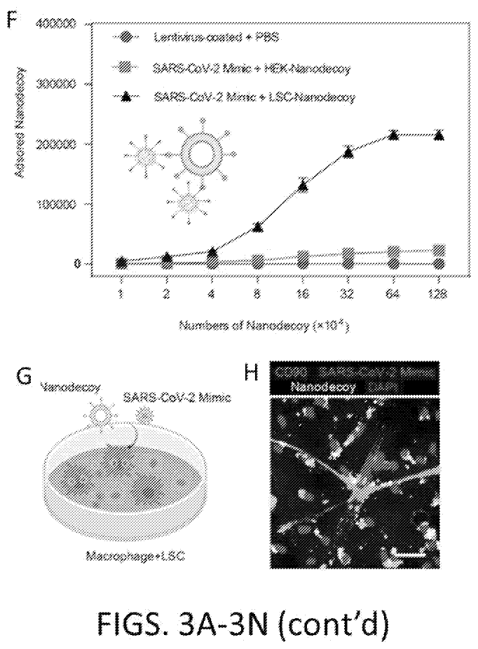

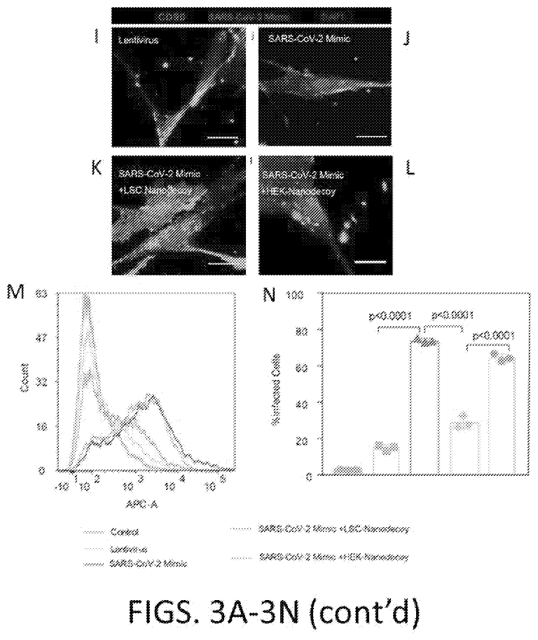

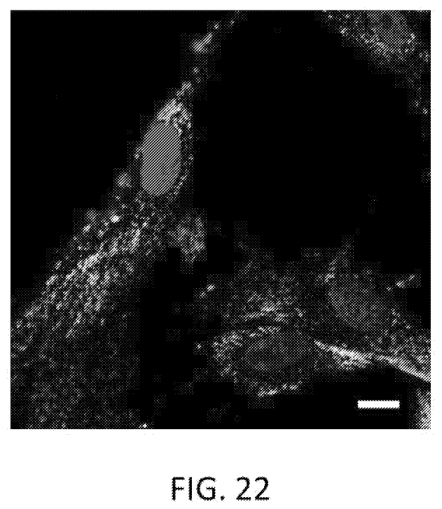

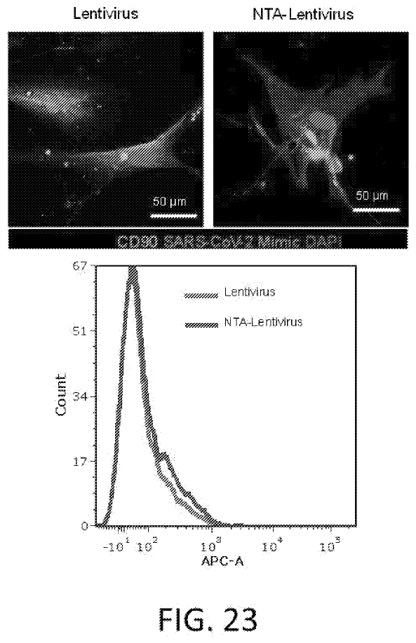

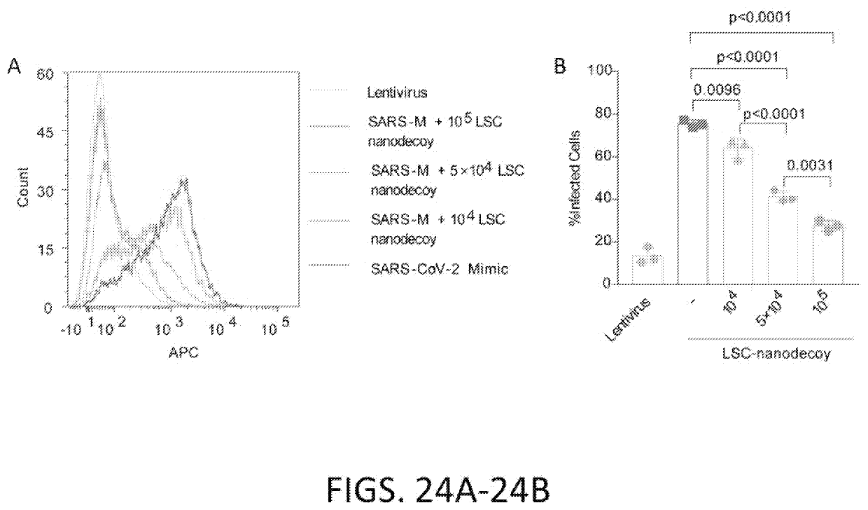

Nanodecoys bind and neutralize SARS-CoV-2 mimics. Next, a spike S1 virus was fabricated to mimic SARS-CoV-2 by modifying a lentivirus without spike S1 to express spike S1 on its surface. Lentiviruses were first modified with Ni nitrilotriacetate (Ni-NTA) (FIG. 3A), and then His-tagged spike S1 was conjugated onto the lentivirus through the interaction of Ni with His tag to generate this SARS-CoV-2 mimic (FIG. 3B). Immunogold labeling was used to confirm spike S1 on the SARS-CoV-2 mimics. TEM imaging visualized the bare lentivirus (FIG. 3C), SARS-CoV-2 mimic (FIG. 3D), and the nanodecoy bound SARS-CoV-2 mimics, shown by the presence of spike S1 on the surface of the modified lentivirus together with the nanodecoy (FIG. 3E), indicating the SARS-CoV-2 mimics were fabricated successfully. Examination indicated that there were approximately 6,900 spike S1 per SARS-CoV-2 mimic virus. It was found that 2.16×10⁵ LSC-nanodecoys could bind 5×10⁵ SARS-CoV-2 mimics (2.31 SARS-CoV-2 mimics per nanodecoy) while HEK293-nanodecoys showed a lower binding efficiency to SARS-CoV-2 mimics, which was owed to the corresponding low ACE2 level (FIG. 3F). This binding interaction is specific since the control lentivirus (without spike S1) had low affinity to LSC-nanodecoys. Macrophages and LSCs were then co-cultured (FIG. 3G), and it was found that SARS-CoV-2 mimics were recognized by LSC-nanodecoys and internalized by macrophages after 4 hours in co-culture (FIG. 3H). The intracellular distribution of the mimics were then examined, and confocal imaging showed some of the mimics within the lysosomes while others resided in the cytoplasm (FIG. 22). In addition, lentiviruses before and after modification had a slight difference in internalization by LSCs (FIG. 23). The inhibiting internalization effect of nanodecoys by LSCs was then examined. Immunocytochemistry (FIGS. 3I-3L) and flow cytometry (FIGS. 3M-3N) confirmed that LSC-nanodecoys could block the entry of SARS-CoV-2 mimics in host cells, but HEK293-nanodecoys could not. Naïve lentiviruses were not efficient in entering lung cells (14.8% infection rate) (FIG. 3I). However, spike S1 modified lentiviruses (SARS-CoV-2 mimics) promoted entry into host cells efficiently (73.8% infection rate) (FIG. 3J), whereas compared with HEK-nanodecoys, LSC-nanodecoys significantly decreased the internalization of SARS-CoV-2 mimics (from 73.8% to 28.8%) (FIGS. 3K-3L). In addition, the dose-dependent blocking effect by LSC-nanodecoys was investigated. Flow cytometry analysis showed that increasing doses of LSC-nanodecoys blocked more virus entry into lung cells in a dose-dependent manner (FIG. 24). Together these results suggest the nanodecoys could protect the host cells from infection by SARS-CoV-2 mimics.

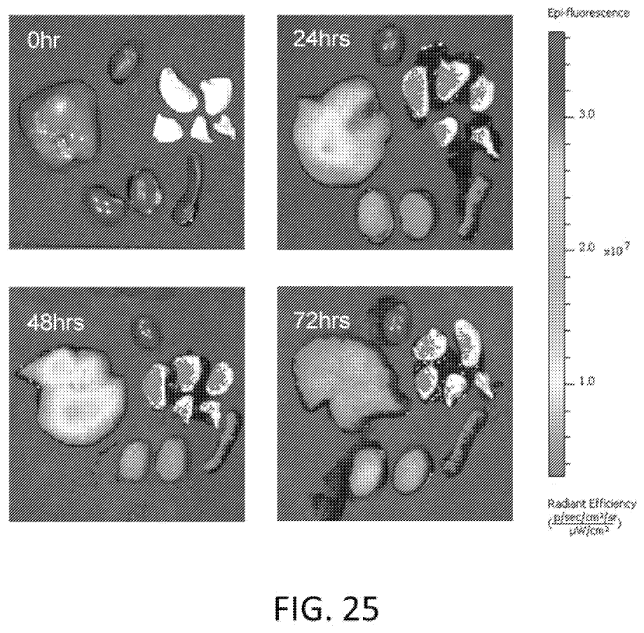

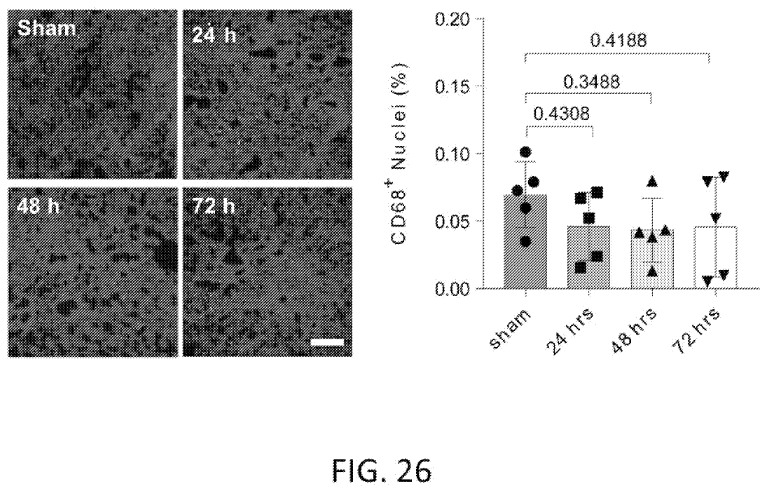

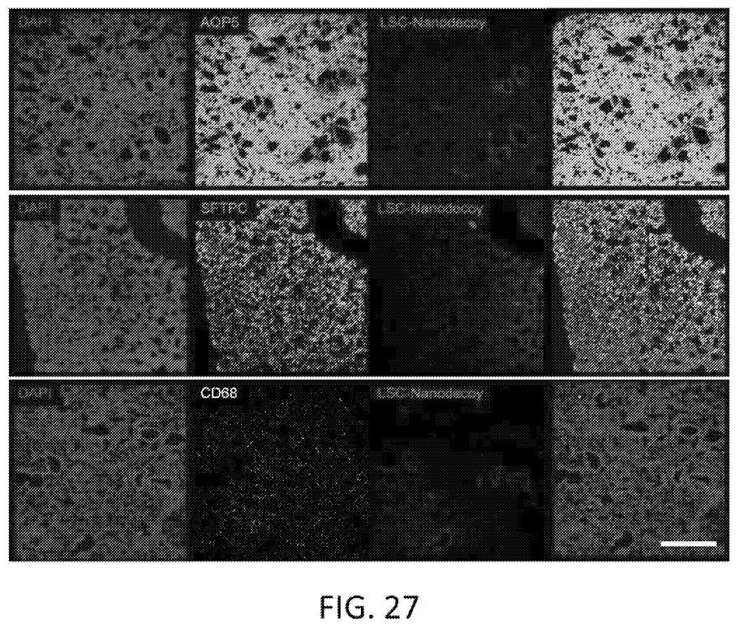

The retention and biodistribution of LSC-nanodecoys in mice after inhalation was then examined. DiD-labeled nanodecoys were administered to mice by inhalation using a commercially available portable nebulizer for clinical relevance at a dose of 1×10¹⁰ nanodecoys per kg of body weight (FIG. 4A). As shown in FIGS. 4B-4C and FIG. 25, nanodecoys could still be found in the lungs 72 hours post a single inhalation treatment. In addition to the lungs, the nanodecoys were also detected in the liver, kidney, and spleen, indicating clearance via the reticuloendothelial system as well as the metabolization of the nanodecoys through the body. Moreover, inhalation of nanodecoys had no significant effect on CD68⁺ macrophage infiltration, indicating their biosafety (FIG. 26). Even though some nanodecoys co-localized with APQ5⁺ (type I) and SFTPC⁺ (type II) cells (FIG. 4D and FIG. 27), the majority of nanodecoys were co-localized in macrophages (FIG. 4E) after 24 hours in vivo.

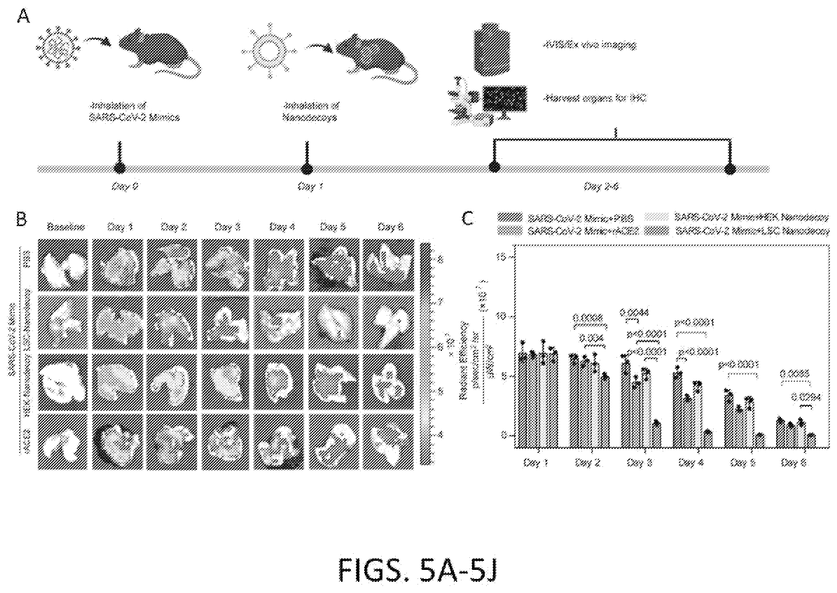

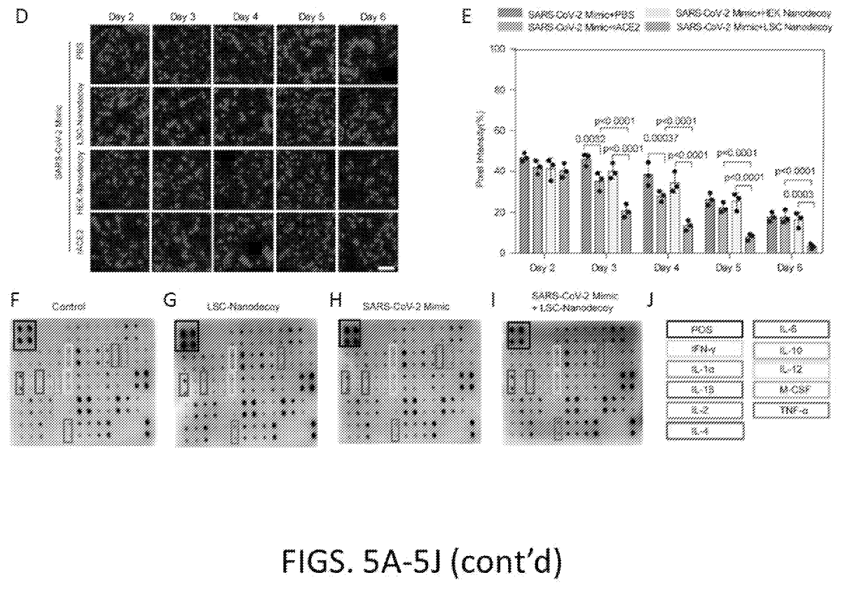

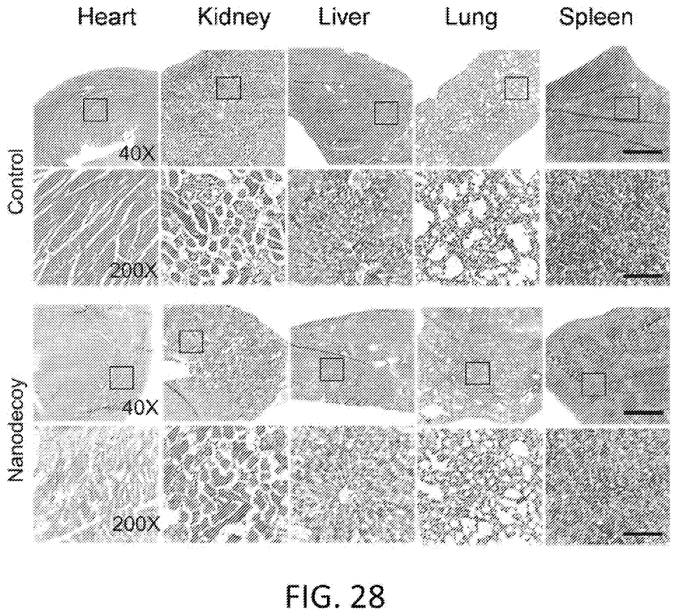

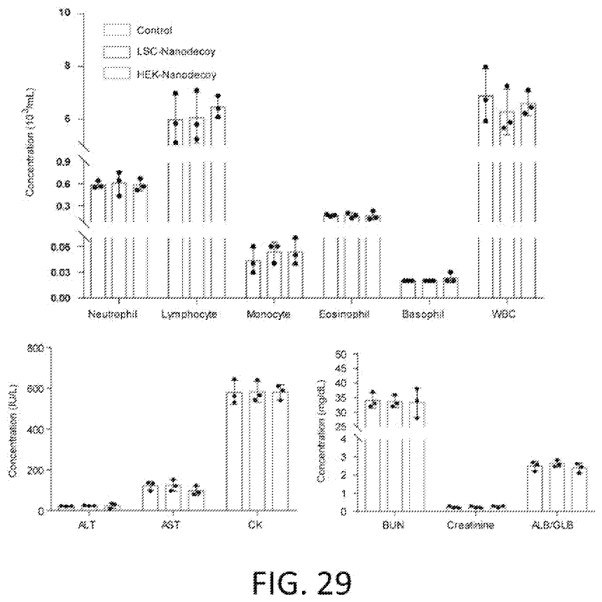

Experiments were performed to test whether inhaled LSC-nanodecoys could accelerate the clearance of SARS-CoV-2 mimics in a mouse model (FIG. 5A). To mimic infection in human patients, mice were allowed to receive the SARS-CoV-2 mimics before initiating administration of the therapeutic nanodecoys. Since treatment started 24 hours post viral exposure, not all of the SARS-CoV-2 mimics were intracellular; therefore, nanodecoys could block the viral mimics from entering the cells further. As for the intracellular SARS-CoV-2 mimics, the nanodecoys that were internalized by cells could capture them, avoiding further infection. Ex vivo imaging (FIGS. 5B-5C) indicated that the amounts of SARS-CoV-2 mimics were significantly reduced following inhalation of LSC-nanodecoys. Inhalation of the freeform of rACE2 and HEK293-nanodecoy were found to be ineffective. Confocal microscopy confirmed that inhalation of LSC-nanodecoys accelerated the clearance of SARS-CoV-2 mimics (FIGS. 5D-5E). Cytokine array analysis (FIGS. 5F-5G) suggested that nanodecoy inhalation did not elevate pro-inflammatory cytokines as compared to the control group. Furthermore, H&E staining of all major organs, hematology, and biochemical parameters indicated no apparent abnormality or adverse effects with LSC or HEK293 nanodecoy inhalation (FIGS. 28-29).

### Example 4

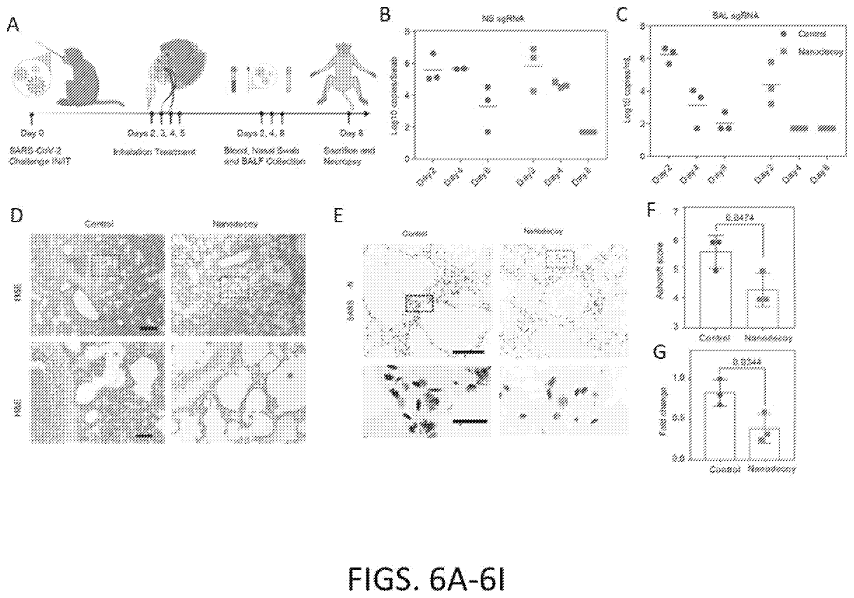

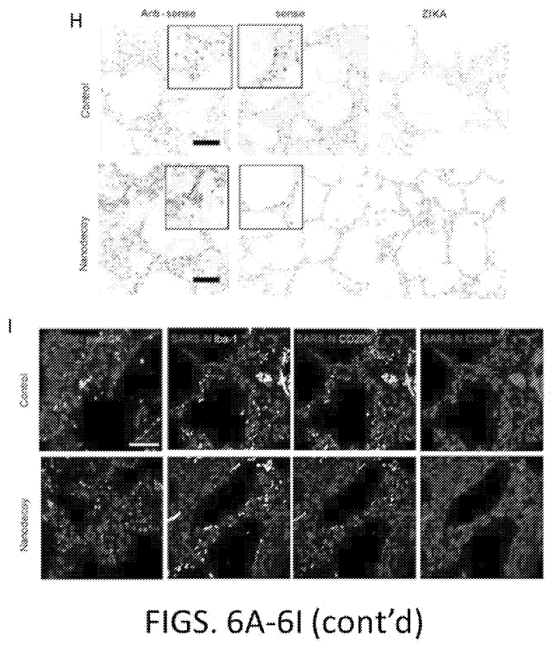

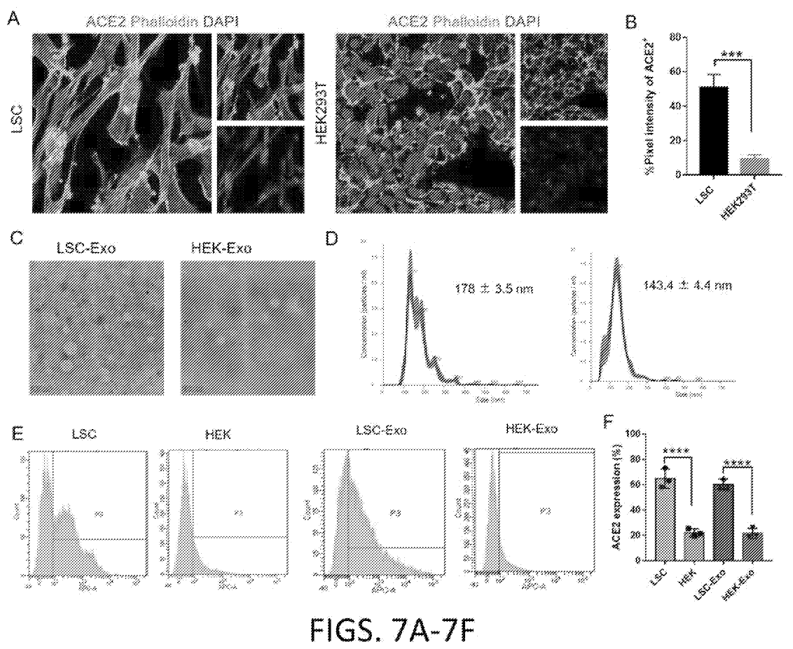

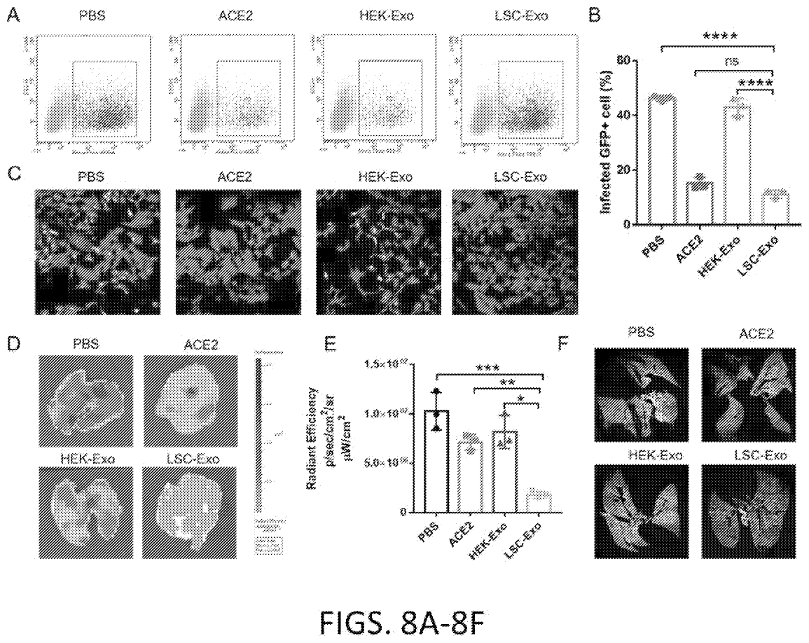

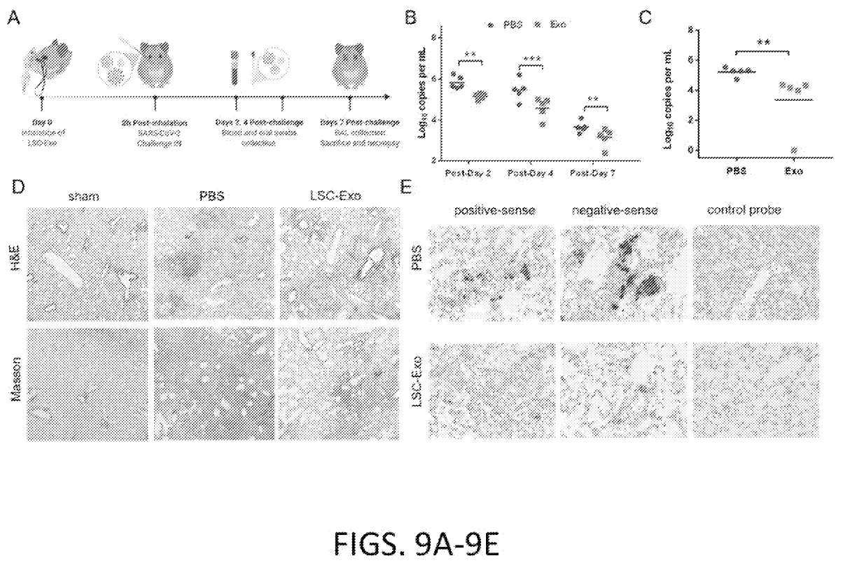

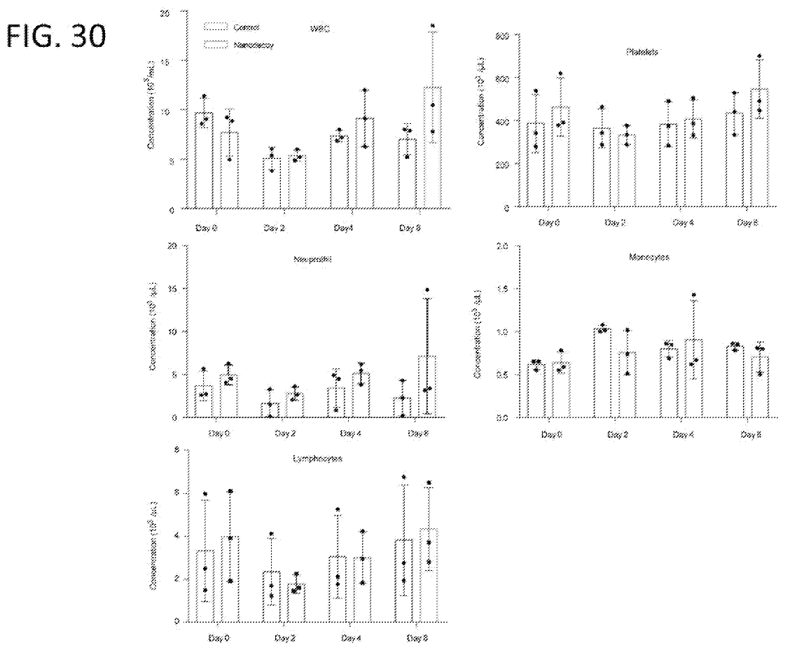

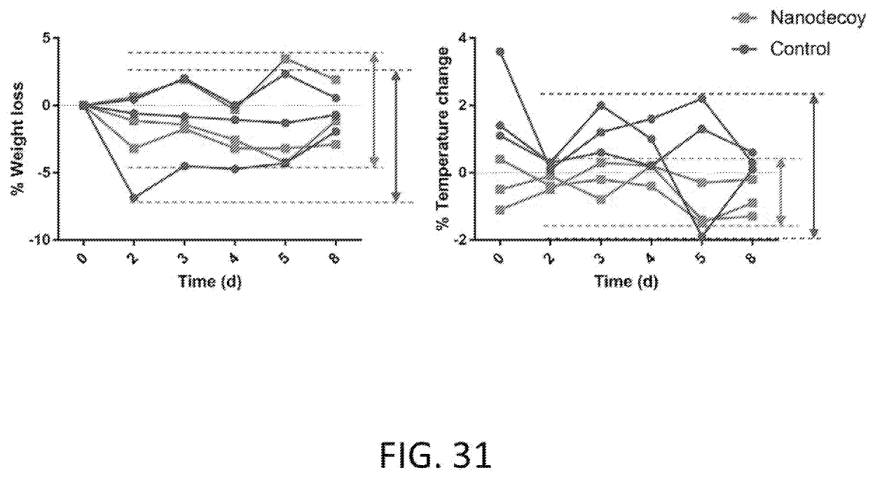

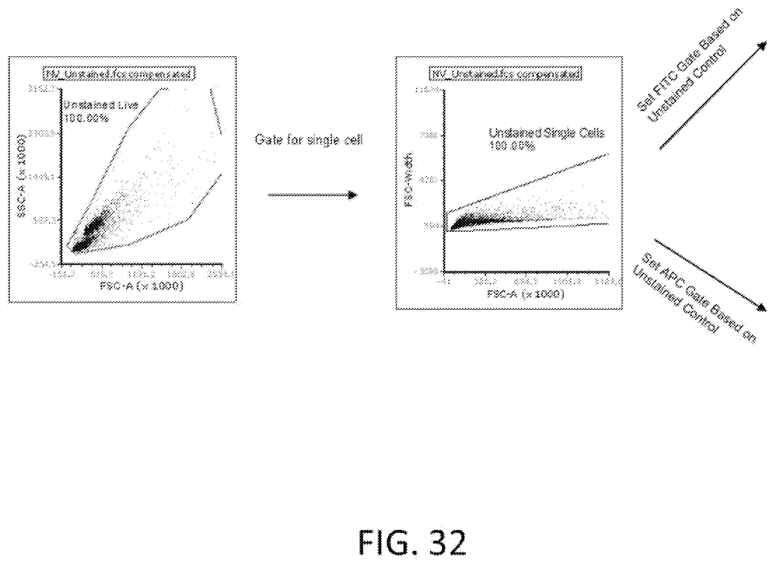

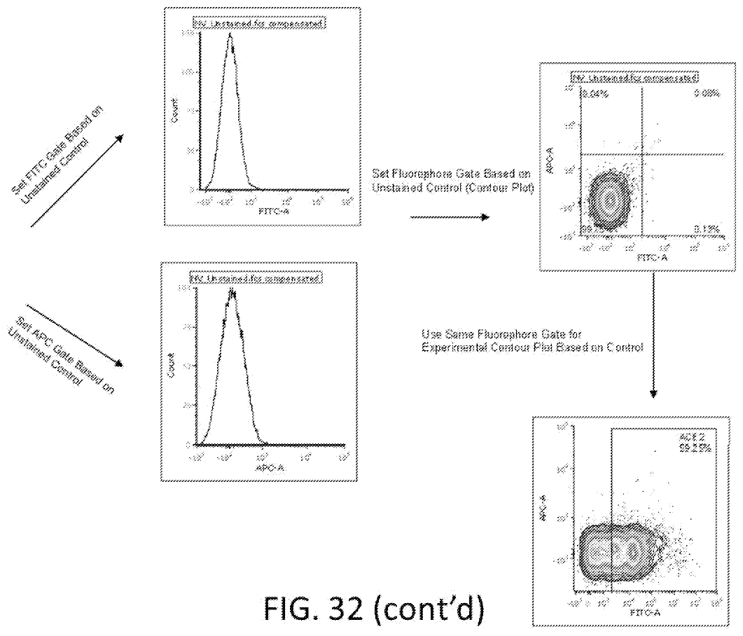

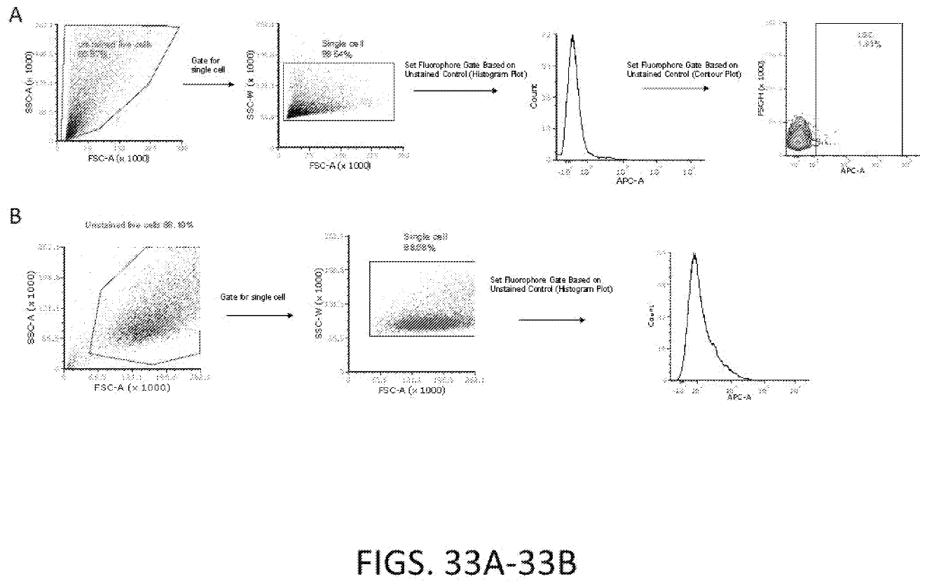

Nanodecoy therapy in SARS-CoV-2 Infected nonhuman primates. A pilot nonhuman primate study was performed to evaluate the safety and preliminary therapeutic efficacy of LSC-nanodecoys. The macaque model can recapitulate many clinical symptoms of SARS-CoV-2 infection and shows a robust viral replication in the upper and lower respiratory tracts. Six cynomolgus macaques were challenged with SARS-CoV-2 by intranasal and intratracheal routes (FIG. 6A). Following challenge, the animals were randomized into two treatment arms: inhalation of PBS or LSC-nanodecoys (at a dose of 10¹⁰ particles per kg of body weight) at days 2, 3, 4, and 5 post-challenge. Viral loads in bronchoalveolar lavage (BAL) and nasal swabs (NS) were assessed by RT-PCR specific for viral subgenomic RNA (sgRNA, indicative of virus replication). As a result, high levels of sgRNA were observed in the control animals with a median peak of 6.243 log₁₀ RNA copies/mL in BAL and a median peak of 5.595 log₁₀ RNA copies/swab in NS on day 2 (FIGS. 6B-6C). sgRNA levels dramatically decreased in nanodecoy-treated animals, with <1.70 log₁₀ reductions of median peak sgRNA in both BAL and NS on day 8 following the challenge. Although sgRNA levels declined in both control and LSC-nanodecoy groups over time, LSC-nanodecoy treatment induced more rapid virus clearance. Negligible difference was observed between the two groups' hematology parameters (FIG. 30). Interestingly, the temperature and body weight fluctuations in the LSC-nanodecoy group were not as drastic as those in control-treated animals (FIG. 31).

At the end of the study, lung tissues of infected cynomolgus macaques were collected and evaluated by histopathology. On day 8 following challenge, multifocal regions of inflammation and evidence of viral pneumonia—including expansion of alveolar septae with mononuclear cell infiltrates, consolidation, and edema—were observed (FIG. 6D). Notably, LSC-nanodecoy treatment significantly reduced the numbers of polymorphonuclear cells and neutrophils as compared with the control group. In addition, Ashcroft score analysis revealed that LSC-nanodecoy treatment significantly decreased lung fibrosis (FIG. 6F). To detect and visualize the virus in lung tissues, SARS nucleocapsid protein (SARS-N) expression was evaluated by immunohistochemistry (IHC) staining. As shown in FIGS. 6E and 6G, multifocal positive pneumocytes and alveolar septa were present in control-treated animals. In contrast, the levels of SARS-N protein were decreased substantially with the LSC-nanodecoy treatment. In addition, SARS-CoV-2 viral RNA (vRNA) was evaluated by in situ RNA hybridization (RNAscope). Compared to the control group, the levels of both positive-sense and negative-sense vRNA were diminished after LSC-nanodecoy treatment (FIG. 6H), indicative of the reduction of viral replication. The distribution of SARS-CoV-2 in lung tissue was assessed by co-staining SARS-N and pan-cytokeratin (pan-CK, to identify epithelial cells). It was found that virus-infected cells greatly overlapped with pan-cytokeratin (pan-CK)-positive cells (FIG. 6I suggesting that they were alveolar epithelial cells. Additionally, foci of virus-infected cells were frequently associated with activated Iba-1⁺ (ionized calcium binding adaptor as a pan-macrophage marker), CD68⁺ (monocyte/macrophage marker), and CD206⁺ (macrophage marker) macrophages (FIG. 6I). Consistent with IHC and RNAscope analysis, immunofluorescence results indicated that nanodecoys could decrease virus levels in lung tissues.

### Example 5

Superior lung biodistribution of exosomes over liposomes after inhalation. Lipid nanoparticles (NPs) have been widely used for RNA vaccine delivery in response to the pandemic of COVID-19. For example, mRNA-1273 vaccine (Moderna), BNT162b1 (BioNTech and Pfizer), and ARCoV mRNA vaccine (Academy of Military Medical Sciences, Suzhou Abogen Biosciences and Walvax Biotechnology) are all lipid-nanoparticle-formulated RNA vaccines. Here, experiments were conducted to determine the biodistribution and retention of NPs (LSC exosomes or liposomes) in the murine lung. Red fluorescent protein (RFP) was loaded into LSC-Exo (RFP-Exo) and commercially-available liposomes (RFP-Lipo) via electroporation, for ex vivo imaging and microscopic visualization. The distribution and fluorescence intensity of RFP-Exo were compared with the gold-standard delivery vesicle RFP-Lipo. RFP-Exo and RFP-Lipo were nebulized to healthy CD1 mice which were sacrificed 4 or 24 hours post NP administration (FIG. 10A). Ex vivo imaging (FIG. 10B) and analysis (FIG. 10C) of the whole lung showed the greatest integrated density of NPs in mice who received RFP-Exo and were sacrificed after 24 hours. Significantly more exosomes are retained and distributed throughout the lung than liposomes (FIGS. 10D-10E). Significantly more exosomes reach the trachea than liposomes, but both NPs diffuse over time (FIG. 10F). Exosome biodistribution 4 hours post administration is the highest in the bronchioles (FIG. 10G), with the parenchyma starting to show exosome signal after 24 hours (FIG. 10H). Significantly less liposomes reach the bronchioles (FIG. 10G) and diffuse into the parenchyma (FIG. 10H), suggesting faster degradation and/or systemic clearance of liposomes in the lung. To verify if antigen presenting cells (APCs) can uptake these NPs, immunohistochemistry was performed (FIG. 10I) on parenchymal lung sections and quantified RFP+ NP uptake by CD11b+ APCs. More APCs are present and uptake exosomes than liposomes (FIG. 10J). Because of exosomes' excellent retention in the lung, as well as enhanced targeting to APCs, exosomes were used as the backbone of the VLPs.

### Example 6

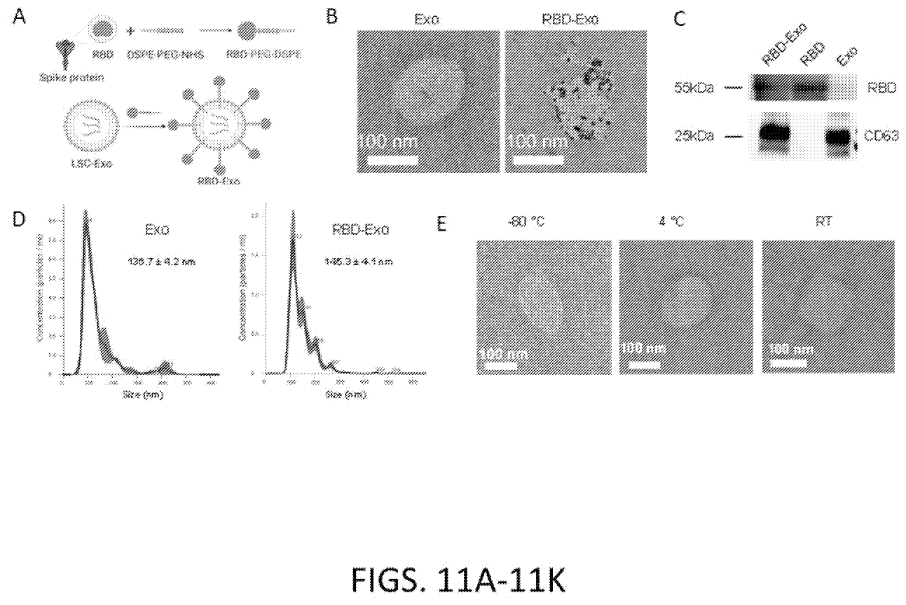

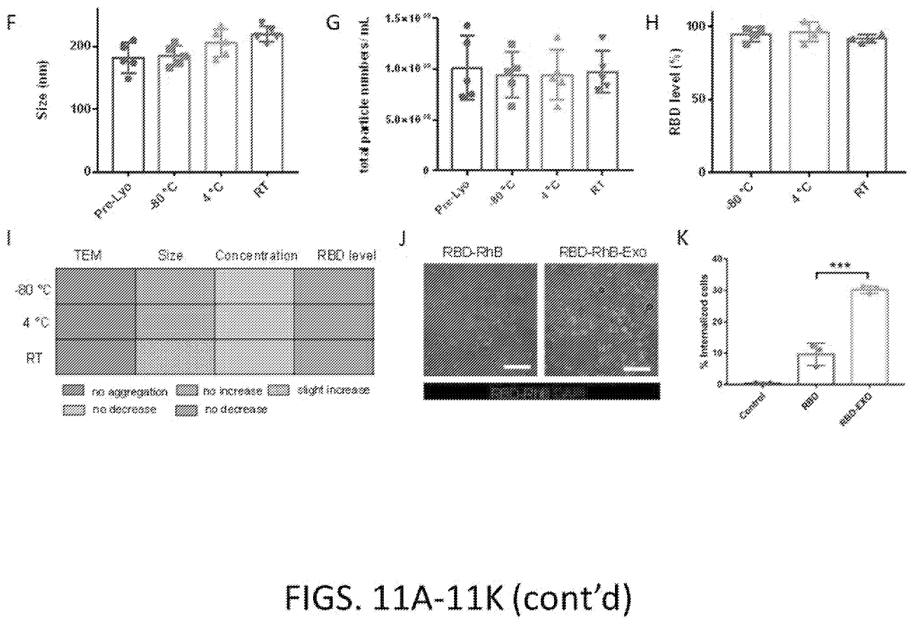

Fabrication and characterization of RBD-Exo VLPs. RBD antigens were conjugated onto the LSC-Exo surface using a [1,2-Dierucoyl-sn-glycero-3-phosphoethanolamine-poly(ethylene-glycol)-n-hydroxysuccinamide] (DSPE-PEG-NHS) linker according to previous methods (FIG. 11A). Next, the binding capacity was optimized to 0.52 μg RBD per 10¹⁰ exosomes. Correspondingly, approximately 892 antibody molecules could bind to each individual VLP. Naïve Exo and RBD-Exo were further characterized using transmission electron microscopy (TEM). Gold nanoparticles were conjugation to anti-RBD antibodies to confirm the presence of RBD on the exosome surface (FIG. 11B). Immunoblotting on RBD-Exo, RBD, and Exo lysate further demonstrated RBD presence in RBD-Exo and RBD, not in Exo control (FIG. 11C). Likewise, exosomal marker CD63 was found to be presented in RBD-Exo and Exo, not in free RBD control (FIG. 11C). In addition, nanoparticle tracking analysis revealed RBD decoration slightly increased the average diameter of exosomes (FIG. 11D). Those compound data confirmed the successful production of RBD-Exo VLPs.

### Example 7

RBD-Exo VLP vaccine is room temperature stable. Experiments were conducted to test whether RBD-Exo VLPs were stable at room temperature storage. RBD-Exo VLPs were lyophilized and stored at −80° C., 4° C., or room temperature (RT) for 21 days (3 weeks). After re-hydration, the morphology and structures of RBD-Exo were well preserved, as indicated by TEM images (FIG. 11E). Their sizes and concentrations were determined using nanoparticle tracking analysis, respectively. As shown in FIGS. 11F-11G, room temperature storage had minimal impact on VLPs' size and concentration. Moreover, the numbers of RBD on RBD-Exo remained un-changed after storage (FIG. 11H). Collectively, those data suggested that RBD-Exo have high physical and antigenic stability at all temperatures (FIG. 11I), superior to other reported vaccines (Table 1).

TABLE 1
Comparison of COVID-19 vaccines.

| | | Vaccine | | Administration | | | |
|---|---|---|---|---|---|---|---|
| Candidate | Manufacturer | type | Vector | route | Doses | Storage | Shipping |
| mRNA- | Moderna | mRNA | liposome | Intramuscular | 2X | −20° C. for 6 | −20° C. |
| 1273 | | | nanoparticles | infection | [symbol] | months 4° C. for 30 days | Dry ice |
| BNT162b1 | Pfizer- | mRNA | liposome | Intramuscular | 2X | −70° C. | −70° C. |
| | BioNTech | | nanoparticles | infection | [symbol] | | Special thermo box with dry ice |
| AD1222 | Oxford | viral vector | adenovirus | Intramuscular | 2X | 2-8° C. | Cold packs |
| | University | (genetically modified virus) | | infection | [symbol] | | |
| RBD-Exo | From the | VLP | exosome | Inhalation | 2X | 4° C. or RT for | 4° C. |
| VLP | present study | | | | [symbol] | at least 21 days | Cold packs |

### Example 8

Internalization of RBD-Exo VLPs by macrophages. To effectively elicit immune response, VLPs need to be recognized and internalized by antigen presenting cells (APCs) such as macrophages. RBD and RBD-Exo were labeled using NHS-Rhodamine (RHS-RhB) and then co-cultured with APCs (murine macrophage RAW264.7 cells). Confocal laser scanning microscopy (CLSM) revealed enhanced RBD-Exo internalization by RAW264.7 cells as compared to free RBD (FIG. 11J). This indicated that exosomes as a carrier enhanced cellular uptake of RBD by APCs. Flow cytometry confirmed the results of CLSM (FIG. 11K).

### Example 9

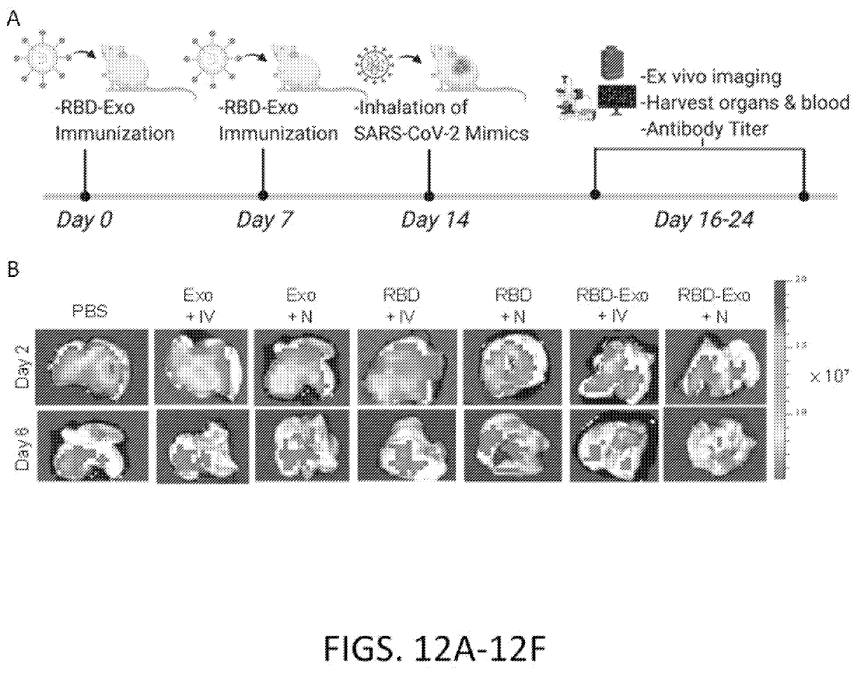

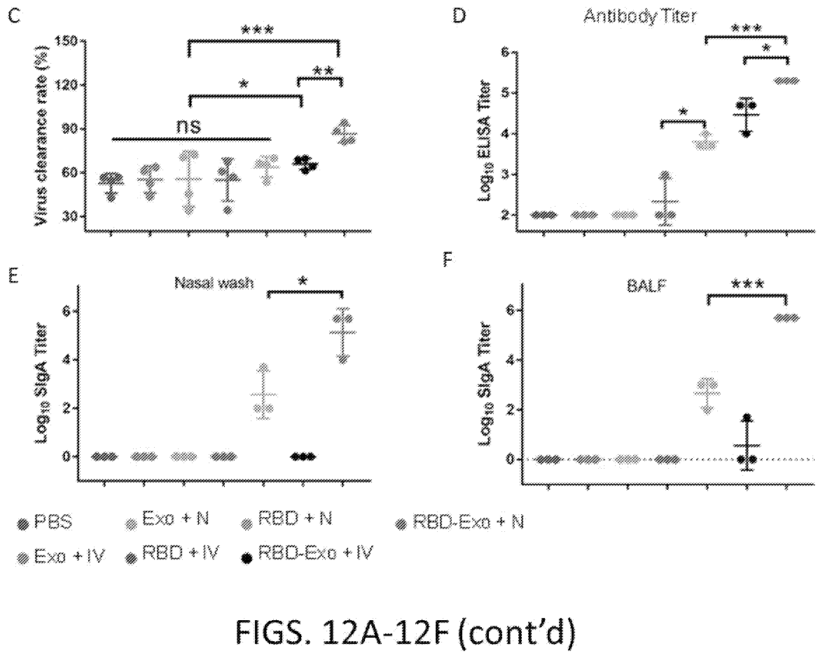

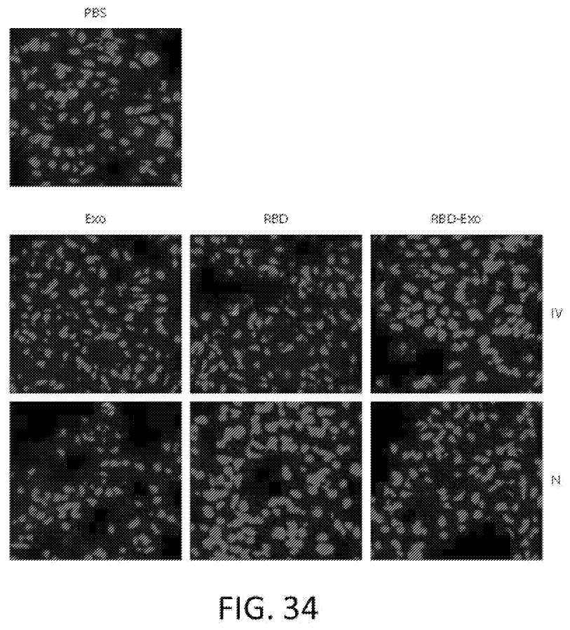

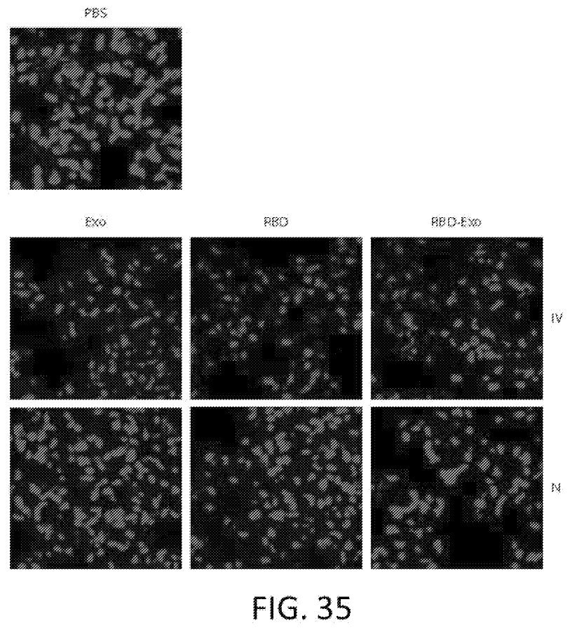

RBD-Exo VLP vaccination generates antibodies to clear SARS-CoV-2 mimics in mice. CD1 mice were randomized to receive two vaccinations two weeks apart for placebo (PBS), control Exo, free RBD, or RBD-Exo via either intravenous (IV) injection or inhalation (nebulization). 7 days after the second vaccination, the mice were challenged with SARS-CoV-2 mimics (labeled by AF647) via intratracheal delivery (FIG. 12A). Mice were sacrificed 2 and 7 days post challenge and lung tissues were imaged using in vivo imaging system (IVIS) to visualize the clearance of SARS-CoV-2 mimics (FIG. 12B). Lung sections were prepared and stained for CLSM imaging. IVIS (FIG. 12C) and CLSM (FIGS. 34-35) suggested that RBD-Exo VLP vaccinations accelerated the clearance of SARS-CoV-2 mimics. Furthermore, nebulization administration across all groups induced more rapid clearance of SARS-CoV-2 mimics than IV injection, suggesting that nebulization is a more targeted and potent delivery strategy of RBD vaccines. An enzyme-linked immunosorbent assay (ELISA) revealed that inhalation of RBD-Exo VLPs induced the most neutralizing antibodies against RBD (FIG. 12D) in the mouse sera.

### Example 10

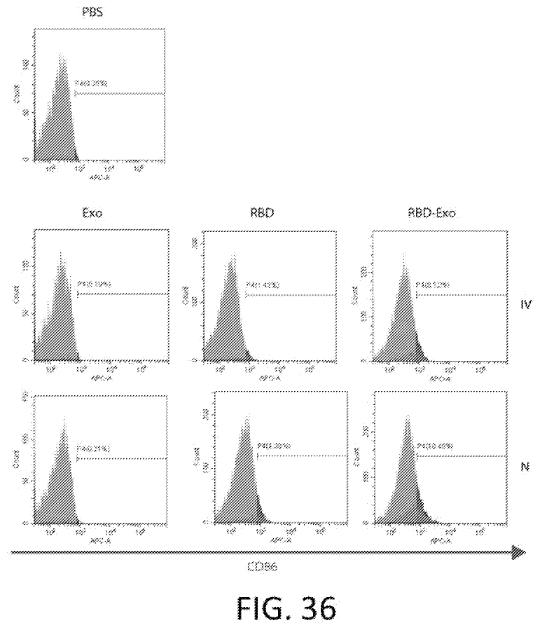

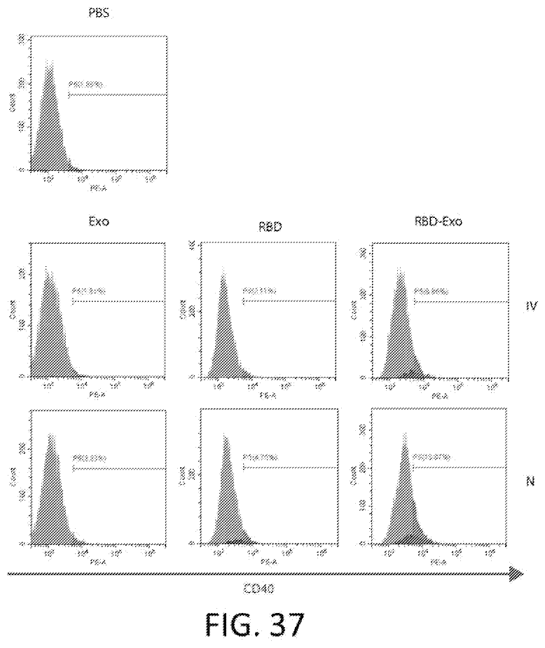

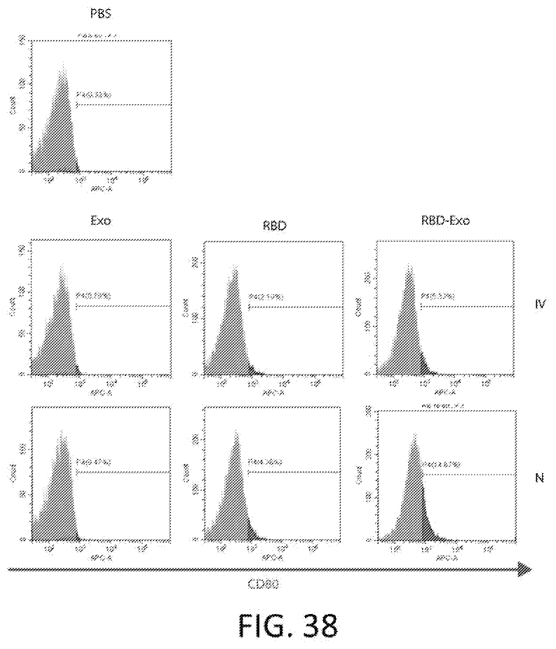

RBD-Exo VLP vaccination induces mucosal immune response. Because the mucosa is the primary entry route of pathogens, the host immune system provides a dynamic immunologic barrier through antigen-specific SIgA responses that play a key role in preventing pathogen invasion. To evaluate mucosal immune response, SIgA antibodies against RBD were measured from nasopharyngeal lavage fluid (NPLF) and bronchoalveolar lavage fluid (BALF) from the mice. ELISA revealed that inhalation of RBD-Exo VLPs produced the highest amount of SIgA antibodies in NPLF (FIG. 12E) and BALF (FIG. 12F). Interestingly, only a negligible amount of SIgA antibodies was found in animals received vaccination via IV injection. Viral antigens were presented to APCs like dendritic cells (DCs) for further immunological response and protection against the pathogen. Murine splenocytes were isolated after euthanasia and re-challenged with RBD to assess DC activation via flow cytometry. A greater percentage of DCs were CD86+ (FIG. 36), CD40+ (FIG. 37), and CD80+ (FIG. 38) in splenocytes derived from RBD-Exo inhalation-treated mice, indicating that more DCs were activated. These data suggested that inhalation of RBD-Exo produced both neutralizing antibodies and SIgA responses against RBD, an important antigen of SARS-CoV-2.

### Example 11

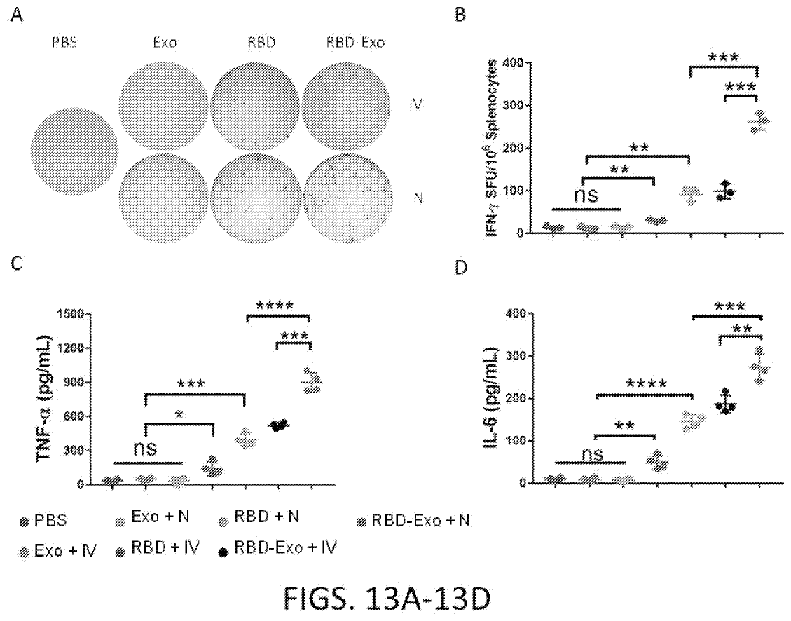

Cellular response against SARS-CoV-2 mimic by RBD-Exo VLP vaccination. Experiments were conducted to evaluate the cellular immune responses and systemic cytokines in vaccinated mice. Enzyme-linked immune absorbent spots (ELISpots) against IFN-γ were performed on splenocytes from animals in each treatment group (FIG. 13A). RBD and RBD-Exo vaccinations induced significantly greater IFN-γ secretion after re-stimulation with RBD (FIG. 13B). Specifically, RBD-Exo inhalation induced approximately 300 spot-forming units (SFU) per 10⁶ splenocytes, the highest among all groups (FIG. 13B). Furthermore, re-stimulation by RBD induced the highest levels of TNF-α, (FIG. 13C) and IL-6 (FIG. 13D) secretion in animals vaccinated with RBD-Exo inhalation. These compound datasets indicated that RBD-Exo inhalation robustly induced a systemic T cell immune response that can further protect the subjects from viral replication.

### Example 12

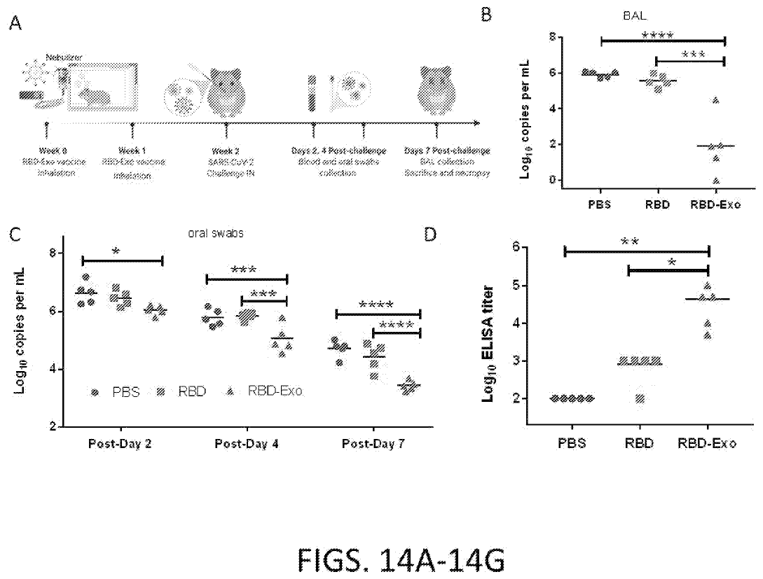

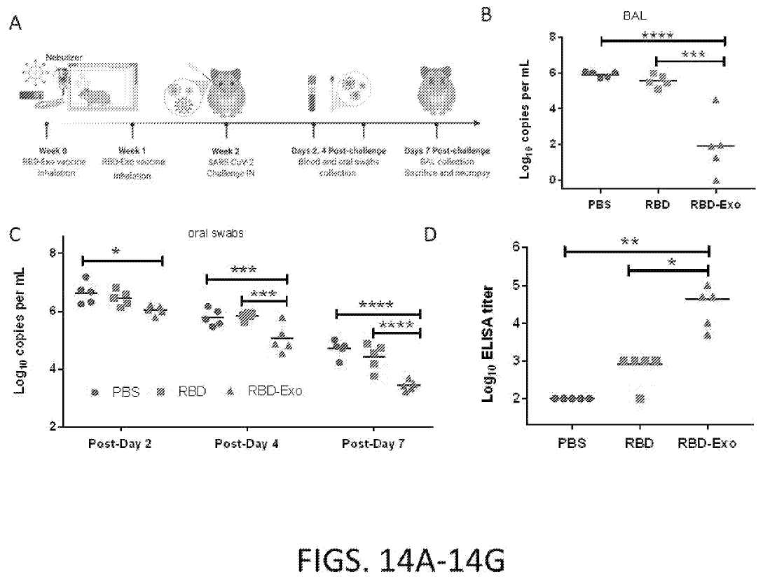

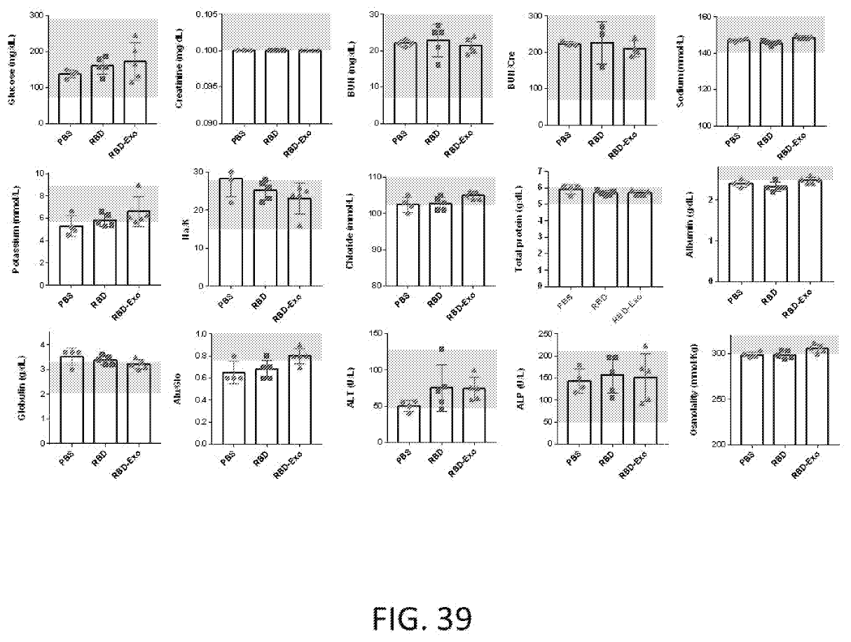

RBD-Exo VLP vaccinations protect hamsters from live SARS-CoV-2 Infection. Experiments were conducted to assess the protective effects of RBD-Exo VLP vaccine in high-dose SARS-CoV-2 infected hamsters, which can replicate severely clinical diseases, accompanied by rapid weight loss and severe lung pathology. After two doses of RBD-Exo vaccination, 15 Syrian golden hamsters (6-8 weeks old) were challenged with 5×10⁵ tissue culture infective dose (TCID₅₀) SARS-CoV-2 by the intranasal and intratracheal routes (FIG. 14A). Viral loads in oral swabs (OS) and bronchoalveolar lavage (BAL) were determined by RT-PCR. As a result, high levels of RNA copies were observed in all three immuned groups of PBS, RBD, RBD-Exo with a median peak of 6.632, 6.454 and 6.042 log₁₀ RNA copies/mL in OS on day 2 (FIG. 14C). RNA levels were dramatically decreased in RBD-Exo-immuned animals, with 3.43 log₁₀ reductions of median peak RNA in OS on day 7 following the challenge. Consistent with the OS results, BAL viral load was approximately 1.942 log₁₀ RNA copies/mL in RBD-Exo immunization group, which was much lower than PBS- (5.916) and RBD- (5.548) treated groups (FIG. 14B). Furthermore, RBD-Exo vaccinations elicited 10-100 folder higher median ELISA titers as compared to RBD vaccinations (FIG. 14D). Clinical chemistry and hematological parameters of hamsters vaccinated with RBD-Exo were in normal range (FIG. 39). Hamsters were assessed by histopathology on days 7 after virus challenge. Hematoxylin and eosin (H&E) staining revealed that severe pulmonary lesions with marked inflammatory infiltrates and multifocal dense nodular with alveolar wall thickening in hamsters received with PBS vaccinations (FIG. 14E). Conversely, the pulmonary alveolus were highly visible in EBD-Exo vaccination group as well as the numbers of polymorphonuclear and neutrophils were significantly reduced (FIG. 14E). Masson trichrome staining and Ashcroft score analysis revealed that RBD-Exo immunization significantly decreased resolution of fibrosis by preserving alveolar epithelial structures compared to PBS vaccination or RBD vaccination (FIGS. 14F-14G).

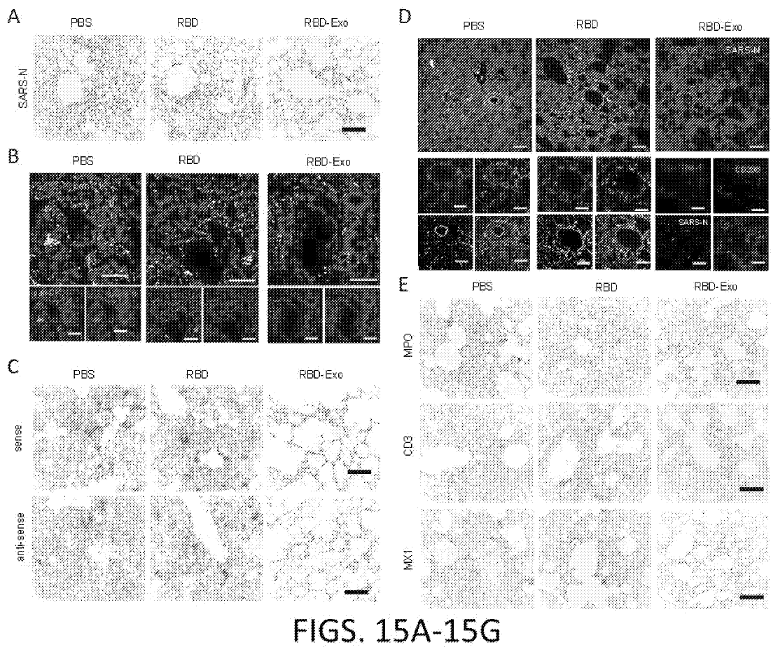

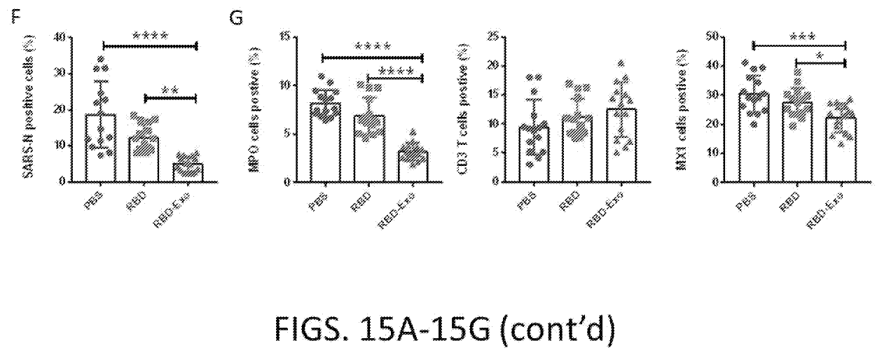

To visualize the virus in lung tissues, the expression and distribution of SARS nucleocapsid protein (SARS-N) were evaluated. As shown in FIG. 15A, multifocal positive pneumocytes and alveolar septa were presented in PBS-treated animals. And these viral antigen positive cells frequently co-stained with pan-cytokeratin (pan-CK, to identify epithelial cells), further confirming that they were alveolar epithelial cells (FIG. 15B). Of special note, the level of SARS-N protein in lung was decreased substantially with RBD-Exo vaccinations (FIG. 15F). Furthermore, SARS-CoV-2 viral RNA (vRNA) in lung was determined by in situ RNA hybridization (RNAscope). Compared to PBS or RBD vaccination, the levels of both positive-sense and negative-sense vRNA were dramatically reduced in RBD-Exo immunization group (FIG. 15C), indicative of the reduction of viral replication by anti-RBD antibodies neutralization. It was found that foci of virus infected cells were frequently associated with large inflammatory infiltrates of Iba-1⁺ (ionized calcium binding adaptor as a pan-macrophage marker) and CD206⁺ (macrophage marker (FIG. 15D). Additionally, many neutrophils were detected throughout the lung with high expression of neutrophil myeloperoxidase (MPO) in challenged hamsters (FIG. 15E). However, RBD-Exo vaccinations showed the least MPO positive cells in lung (FIG. 15G). Diffuse expression of CD3-positive T lymphocytes were discovered in challenged hamsters (FIG. 15E), which was able to facilitate the rapid clearance of the infected cells. Importantly, the expression of antiviral protein that type 1 interferon response gene MX1 with antiviral activity against a wide range of RNA viruses was significantly decreased in the RBD-Exo vaccinations compared with PBS vaccinations or RBD vaccinations, further validating decreased virus replication owing to highly potent neutralizing antibody induced by the vaccine. Taken together, these histology results strongly indicated that RBD-Exo vaccination could effectively protect hamster lungs from SARS-CoV-2 infection.

What is claimed is:

1. A composition comprising a plurality of nanovesicles derived from a cell comprising at least one cell surface protein capable of binding a virus.

2. The composition of claim 1, wherein cell is a lung spheroid cell (LSC).

3. The composition of claim 1 or claim 2, wherein the at least one cell surface protein comprises Angiotensin-converting enzyme 2 (ACE2), or a derivative or fragment thereof.

4. The composition of claim 3, wherein the ACE2 protein or derivative or fragment thereof is endogenous to the cell.

5. The composition of claim 3, wherein the ACE2 protein or derivative or fragment thereof is exogenous to the cell.

6. The composition of any of claims 1 to 5, wherein the at least one cell surface protein further comprises AQP5, SFTPC, CD68, EpCAM, CD90, and/or MUC5b.

7. The composition of any of claims 1 to 6, wherein the plurality of nanovesicles comprise an average size ranging from about 50 nm to about 1000 nm.

8. The composition of any of claims 1 to 6, wherein the plurality of nanovesicles comprise an average size of about 320 nm.

9. The composition of any of claims 1 to 8, wherein the composition further comprises at least one pharmaceutically-acceptable excipient or carrier.

10. The composition of any of claims 1 to 9, wherein the virus is a coronavirus.

11. The composition of claim 10, wherein the coronavirus is selected from the group consisting of 229E, NL63, OC43, HKU1, MERS-CoV, SARS-CoV, and SARS-CoV-2.

12. The composition of any of claims 1 to 11, wherein the plurality of nanovesicles comprise at least one therapeutic protein, peptide, polypeptide, nucleic acid molecule, polynucleotide, mRNA, siRNA, miRNA, antisense oligonucleotide, drug, or therapeutic small molecule.

13. A method of treating a viral infection comprising administering the composition of any of claims 1 to 12 to a subject in need thereof.

14. The method of claim 13, wherein the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration.

15. The method of claim 13, wherein the composition is administered via nebulization to lung tissue.

16. The method of any of claims 13 to 15, wherein administration of the plurality of nanovesicles reduces viral load in the subject.

17. The method of any of claims 13 to 16, wherein the composition is administered at a dosage ranging from about 1×10⁸ to about 1×10¹² particles per kg of body weight of the subject.

18. A method of generating a plurality of nanovesicles capable of treating a viral infection, the method comprising:
culturing a plurality of lung spheroid cells (LSCs); and
subjecting the plurality of LSCs to an extrusion process to produce the plurality of nanovesicles.

19. The method of claim 18, wherein the extrusion process comprises passing the LSCs through an extruder comprising 5 μm, 1 μm, and 400 nm pore-sized membrane filters.

20. The method of claim 18 or claim 19, wherein the method further comprises purifying and concentrating the plurality of nanovesicles using ultrafiltration.

21. A composition comprising a plurality of exosomes derived from lung spheroid cells (LSCs), wherein the plurality of LSC exosomes comprise:
(i) at least one membrane-associated protein on the surface of the plurality of LSC exosomes; and/or
(ii) at least one antiviral therapeutic agent contained within the plurality of LSC exosomes.

22. The composition of claim 21, wherein the at least one membrane-associated protein on the surface of the plurality of LSC exosomes comprises a viral-specific protein, or a derivative or fragment thereof.

23. The composition of claim 22, wherein the viral-specific protein comprises a Spike protein (S protein), or a derivative or fragment thereof.

24. The composition of claim 22, wherein the viral-specific protein comprises a receptor binding domain (RBD) of a Spike protein (S protein), or a derivative or fragment thereof, capable of binding Angiotensin-converting enzyme 2 (ACE2)

25. The composition of claim 22, wherein the viral-specific protein comprises an antigenic epitope or derivative or fragment thereof capable of stimulating an immune response in a subject.

26. The composition of any of claims 21 to 25, wherein the at least one antiviral therapeutic agent contained within the plurality of LSC exosomes comprises mRNA encoding the S protein.

27. The composition of claim 21, wherein the at least one membrane-associated protein on the surface of the plurality of LSC exosomes comprises a protein capable of binding a virus.

28. The composition of claim 27, wherein the protein capable of binding a virus comprises Angiotensin-converting enzyme 2 (ACE2), or a derivative or fragment thereof.

29. The composition of claim 27 or claim 28, wherein the at least one antiviral therapeutic agent contained within the plurality of LSC exosomes comprises remdesivir, interferon beta-1b, and/or lopinavir-ritonavir.

30. The composition of any of claims 21 to 29, wherein the composition further comprises at least one pharmaceutically-acceptable excipient or carrier.

31. A method of preventing a viral infection comprising administering the composition of any of claims 21 to 30 to a subject.

32. The method of claim 31, wherein the composition is administered orally, parenterally, intramuscularly, intraperitoneally, intravenously, intracerebroventricularly, intracisternally, intratracheally, intranasally, subcutaneously, via injection or infusion, via inhalation, spray, nasal, vaginal, rectal, sublingual, or topical administration.

33. The method of claim 32, wherein the composition is administered via nebulization to lung tissue.

34. The method of any of claims 31 to 33, wherein the virus is a coronavirus.

35. The method of claim 34, wherein the coronavirus is selected from the group consisting of 229E, NL63, OC43, HKU1, MERS-CoV, SARS-CoV, and SARS-CoV-2.
